(19) United States
(12) Patent Application Publication
(10) Pub. No.: US 2026/0306482 A1
(43) Pub. Date: Oct. 8, 2026

(54) METHODS OF TREATING DISEASES AND DISORDERS ASSOCIATED WITH FOXP3 EXPRESSION

(71) Applicant: Northwestern University, Evanston, IL (US)

(72) Inventors: Deyu Fang, Evanston, IL (US); Amy Tang, Evanston, IL (US); Xiaoyu Zhang, Evanston, IL (US); Beixue Gao, Evanston, IL (US)

(21) Appl. No.: 19/636,687

(22) Filed: Apr. 1, 2026

Related U.S. Application Data
(60) Provisional application No. 63/782,552, filed on Apr. 2, 2025.

(51) Int. Cl. A61K 31/495; A61K 31/4436; A61K 31/4468; A61K 31/454; A61K 45/06; A61P 35/00; A61P 37/02

(52) U.S. Cl. CPC A61K 31/495; A61K 31/4436; A61K 31/4468; A61K 31/454; A61K 45/06; A61P 35/00; A61P 37/02

(57) ABSTRACT

Disclosed herein are methods of treating diseases and disorders associated with FoxP3 expression by administering the disclosed compounds. The inventors discovered that several electrophilic compounds are specific degraders of FoxP3. Also disclosed are methods of inducing degradation of FoxP3 in a cell comprising contacting the cell with the disclosed compounds.

### CROSS-REFERENCE TO RELATED APPLICATIONS

The present application claims priority to U.S. Provisional Patent Application No. 63/782,552 that was filed Apr. 2, 2025. The entire contents of which are hereby incorporated by reference.

### STATEMENT REGARDING FEDERALLY SPONSORED RESEARCH

This invention was made with government support under grant numbers CA287701, GM105538 and GM149439 awarded by the National Institutes of Health. The government has certain rights in the invention.

### REFERENCE TO A SEQUENCE LISTING

A Sequence Listing accompanies this application and is submitted as an xml file of the sequence listing named “702581_02764.xml” which is 4,784 bytes in size and was created on Mar. 20, 2026. The sequence listing is electronically submitted via Patent Center and is incorporated by reference herein in its entirety.

### FIELD OF THE INVENTION

The disclosed technology is generally directed to the treatment of cancer and chronic viral infection. More particularly the technology is directed to small molecule degraders that target forkhead box P3 (FoxP3) which is a lineage-defining transcription factor required for function of T regulatory cells (Tregs).

### BACKGROUND OF THE INVENTION

T regulatory cells (Tregs) are a subset of, typically CD4⁺, T cells which are required to maintain peripheral immune tolerance and prevent autoimmunity through their immunosuppressive functions. However, in many different types of cancer, Tregs suppress the anti-cancer immune response in multifaceted methods including but not limited to competition for key cytokines such as IL-2, expression of inhibitory receptors such as CTLA-4 and PD-L1, and release of inhibitory cytokines such as IL-10. Moreover, Tregs are an important factor in the lack of complete response or disease regression after immune checkpoint blockade due to multiple suppressive mechanisms or reinvigoration of suppressive Tregs alongside cytotoxic T cells. As a result, Tregs have come under scrutiny as a major target for tumor therapeutics. FoxP3⁺ Treg depletion has been shown to be beneficial to animals bearing tumors; in particular, CD4 or CD25 monoclonal antibodies and agents that affect Treg development, recruitment, or function (blocking CTLA4 and PD-1) have been explored. However, these approaches are not Treg-specific, and modest efficacies (that may reflect the co-targeting of activated effector T cells) and transient effects (rebound increases in Treg numbers) have been observed. Therefore, approaches that specifically suppress Treg functions without impairing conventional T cell antitumor immunity are needed.

### BRIEF SUMMARY OF THE INVENTION

Disclosed herein are methods of treating a disease or disorder associated with forkhead box P3 (FoxP3) expression in a subject in need thereof, methods of inducing degradation of forkhead box P3 (FoxP3) in a cell, and small molecule FoxP3 degraders. One aspect is a method of treating a disease or disorder associated with forkhead box protein P3 (FoxP3) expression in a subject in need thereof, the method comprising administering a therapeutically effective amount of a small molecule FoxP3 degrader. Suitably, the disease or disorder associated with FoxP3 expression may be a cell proliferative disease or disorder or a chronic viral infection. The method can decrease T regulatory cell (Treg) FoxP3 protein or decrease glucocorticoid-induced TNFR-related protein (GITR) expression.

Another aspect is a method of inducing degradation of forkhead box protein P3 (FoxP3) in a cell, the method comprising contacting the cell with an effective amount of a small molecule FoxP3 degrader. The cell may be a T regulatory cell (Treg), such as a CD8 or CD4 T cell. The method can decrease glucocorticoid-induced TNFR-related protein (GITR) expression in the cell.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

The patent or application file contains at least one drawing executed in color. Copies of this patent or patent application publication with color drawing(s) will be provided by the Office upon request and payment of the necessary fee.

FIGS. 1A, 1B, 1C, 1D, 1E, 1F, 1G, and 1H show high throughput screening identifies FD03 as a degrader of FoxP3. (FIG. 1A) Schematic of screening experiments and methodology. (FIG. 1B) Chemical structure of FD03. (FIG. 1C) Quantification of HEK293T cells stably expressing FoxP3-Artichoke treated with DMSO control or 5 μM FD03 for 16 hours. (FIG. 1D) Representative immunoblots and quantification of FoxP3 in MT-2 cells treated with varying concentrations of FD03 for 16-18 hours from n=4 independent experiments. Murine Tregs were polarized from CD4+ T cells and treated with DMSO control or varying concentrations of FD03 for 16-18 hours. (FIG. 1E) Assessment of cytotoxicity by viability staining readout by flow cytometry representative of n=4-9 biological replicates. (FIG. 1F) Representative immunoblots and quantification of FoxP3 in treated iTreg cells representative of n=4 biological replicates. (FIG. 1G, FIG. 1H) Primary human CD4+ T cells were polarized into iTregs and treated with DMSO control or varying concentrations of FD03 for 16-18 hours. (FIG. 1G) Assessment of cytotoxicity by viability staining readout by flow cytometry representative of n=4 biological replicates. (FIG. 1H) Representative histogram and quantification of FoxP3 in treated human iTreg cells representative of n=4 biological replicates. All quantified data is presented as mean±s.d.

FIGS. 2A, 2B, 2C, 2D, and 2E show FD03 promotes proteasomal degradation of FoxP3 through STUB1. (FIG. 2A) Representative immunoblot and quantification of MT-2 cells co-treated with DMSO or 20 μM FD03 and MG132. Cells were initially treated with DMSO/FD03 for 16-18 hours; MG132 dosed in for additional 4 hours. (FIG. 2B) Representative immunoblot and quantification of MT-2 cells co-treated with CHX and DMSO or 20 μM FD03. (FIG. 2C) Representative image of FoxP3 ubiquitination in MT-2 cells co-treated with DMSO or 20 μM FD03 and MG132. Anti-FoxP3 was used to immunoprecipitated FoxP3 and interacting proteins; IgG was used as a control followed by immunoblot with the indicated antibodies. IP=immunoprecipitation; WCL=whole cell lysate. Data is representative of n=3 independent experiments. Treatment timing is same as described in (a). (FIG. 2D) Representative immunoblot and quantification of FoxP3-STUB1 interaction in MT-2 cells co-treated with DMSO or 20 μM FD03 and MG132. Anti-FoxP3 was used to immunoprecipitated FoxP3 and interacting proteins followed by immunoblot with indicated antibodies. Quantification was performed by normalizing to respective IP-IB FoxP3 band intensity. Treatment timing is same as described in (a). (FIG. 2E) Representative immunoblot and quantification of wild-type (WT) and STUB1-KO MT-2 cells treated with DMSO, 10 or 20 μM FD03 for 16-20 hours. All quantified data is presented as mean±s.d.

FIGS. 3A, 3B, 3C, 3D, 3E, 3F, 3G, and 3H show FD03 treatment diminishes Treg suppressive capabilities. (FIG. 3A) Representative histograms and quantification of murine Treg markers including FoxP3, GITR (CD357), CD25, PD-L1 (CD274), and CTLA-4 (CD152) following treatment with DMSO control or 10 μM FD03 48 hours after polarization and rest. (FIG. 3B) Representative histogram and quantification of murine CD8 T cell growth in co-culture with treated murine Treg cells. Ratios indicate number of Treg cells to CD8+ (Teff). Division index was calculated using proliferation function in FlowJo. (FIGS. 3C-3H) Representative histograms (DMSO and 10 μM FD03) and quantification of human Tregs treated with DMSO control or varying concentrations of FD03 for 72 hours following polarization. (FIG. 3C) FoxP3; (FIG. 3D) GITR (CD357); (FIG. 3E) CD25; (FIG. 3F) PD-L1 (CD274) 1; (FIG. 3G) CTLA-4 (CD152); and (FIG. 3H) PD-1 (CD279). All data in (FIGS. 3A-3H) is representative of n=6 biological replicates. All data is presented as mean±s.d.

FIGS. 4A, 4B, 4C, 4D, 4E, 4F, 4G, and 4H show FD03 induces FoxP3 degradation and decreases murine nTreg suppressive function in an interferon-gamma-dependent manner. (FIG. 4A) Representative immunoblot and quantification of murine nTregs cells co-treated with DMSO or varying concentrations FD03 with and without 10 ng/ml IFN-γ for 24 hours. (FIG. 4B) Representative histogram and quantification of FoxP3 MFI in murine nTregs co-treated with DMSO or varying concentrations FD03 with and without 10 ng/mL IFN-γ for 48 hours. (FIGS. 4C-4F) Murine nTregs were treated with: DMSO, 20 μM FD03, DMSO and 10 ng/mL IFN-γ, or 20 μM FD03 and 10 ng/mL IFN-γ for 48 hours. Representative histograms (left) and quantifications of indicated protein MFI (right) for (FIG. 4C) GITR (CD357), (FIG. 4D) CD25, (FIG. 4E) PD-L1 (CD274), and (FIG. 4F) CTLA-4 (CD152). (FIG. 4G) Representative histogram and quantification of murine CD8+ T cells co-cultured with nTregs pre-treated DMSO, 10 μM FD03, DMSO and 10 ng/mL IFN-γ, or 10 μM FD03 and 10 ng/mL IFN-γ at a 2:1 Treg: T_{eff} ratio unless otherwise indicated. Division index was calculated using proliferation function in FlowJo. (FIG. 4H) Representative flow plots and quantification of percentage of FoxP3+ cells out of total splenic CD4+ T cells and FoxP3 MFI of FoxP3+ cells in mice treated daily for a total of three times with vehicle control (PBS with DMSO) or 50 mg/kg FD03 by I.P. administration. Data from (FIG. 4A) is indicative of n=3 biological replicates from n=3 independent experiments. Data from (FIGS. 4B-4G) is indicative of n=4 biological replicates from n=1 independent experiment. Data from (FIG. 4H) is indicative of n=3 biological replicates from n=1 independent experiment. All data is presented as mean±s.d.

FIGS. 5A, 5B, 5C, 5D, 5E, 5F, 5G, 5H, 5I, 5J, 5K, 5L, 5M, 5N, 5O, 5P, and 5Q show FD03 treatment ameliorates tumor growth through decreased Treg suppressive function. (FIG. 5A-5B) B16-F10 melanoma cells, (FIGS. 5C-5D) EG7 lymphoma cells, or (FIGS. 5E-5F) MC38 colon carcinoma cells were implanted subcutaneously onto the flank of wild-type mice. Mice were treated daily with vehicle control (PBS with DMSO) or 5 mg/kg FD03 peritumorally on the indicated days when the tumors averaged 100 mm³. (FIGS. 5A, 5C, 5E) Quantification of tumor volume over time. (FIGS. 5B, 5D, 5F) Quantification of tumor mass at time of euthanasia. (FIG. 5G) Quantification of CD45+ cells out of total live cells. (FIG. 5H) Representative flow plots (left) and quantification of Tconv (CD45+CD4+FoxP3−), Treg (CD45+CD4+FoxP3+), and CD8 (CD45+CD8+) out of total live CD45+ cells. (FIG. 5I) Representative flow plots (upper) and quantification (lower) of IFN-γ+CD45+CD4+ cells out of total CD45+CD4+ cells. (FIG. 5J) Representative flow plots (upper) and quantification (lower) of IFN-γ+CD45+CD4+ cells out of total CD45+CD8+ cells. (FIG. 5K) Representative flow plots (upper) and quantification (lower) of GZMB+CD8 cells out of total CD45+CD8+. (FIG. 5L) Representative flow plots (left) of total CD45+CD4+ cells gated on FoxP3+ and quantification (right) of FoxP3 MFI relative to vehicle control in Tregs. (FIG. 5M) Representative histogram (left) and quantification (right) of GITR (CD357) MFI relative to vehicle control in Tregs. (FIG. 5N) Representative histograms (left) and quantification of TIGIT MFI relative to vehicle control and percent TIGIT+ in Tregs. (FIG. 5O) Quantification of Helios MFI relative to vehicle control in Tregs. (FIGS. 5P-5Q) LLC-1 lung carcinoma cells were implanted subcutaneously onto the flank of wild-type mice. Mice were treated at the indicated time points by vehicle or 5 mg/kg FD03 intratumorally (teal arrow) and 100 μg IgG or anti-PD-1 via I.P. administration (black arrow). (FIG. 5P) Quantification of tumor volume foldchange normalized to first day of treatment (DO). (FIG. 5Q) Quantification of tumor weight at time of euthanasia. Data from (FIGS. 5A-5B) is representative of n=12 and n=10 biological replicates for vehicle control and FD03 treatment respectively from n=2 independent experiments; from (FIGS. 5C-5D) is representative of n=8 biological replicates for control and FD03 treatment from n=2 independent experiments; from (FIG. 5E) is representative of n=5 biological replicates from n=1 independent experiment; and from (FIG. 5F) is representative of n=7 and n=8 vehicle control and FD03 treatment respectively from n=2 independent experiments. Data in (FIGS. 5G-5O) representative of n=6 and n=7 biological replicates for PBS vehicle control and FD03 treated respectively from n=3 independent experiments. For % TIGIT+ in (FIG. 5P), n=4 biological replicates from n=2 independent experiments. Data from (FIGS. 5P-5Q) is representative of n=5-6 biological replicates per treatment condition and n=1 experiment. All quantified data is presented as mean±s.d.

FIGS. 6A, 6B, 6C, 6D, 6E, 6F, 6G, and 6H show Systemic FD03 treatment only affects tumoral Treg FoxP3 and controls tumor growth. Wild-type mice were implanted with B16 melanoma cells subcutaneously on the flank. When tumors were palpable, mice were treated daily with either vehicle control of 50 mg/kg of FD03 systemically through I.P. administration. (FIG. 6A) Quantification of tumor volume over time; arrows indicate days that mice were treated with vehicle or FD03. (FIG. 6B) Quantification of tumor mass at time of euthanasia. (FIG. 6C) Quantification of percentage CD45+ cells out of live cells. (FIG. 6D) Quantification (left) and representative flow (right) of T cell populations (Tconv (CD45+CD4+FoxP3−), Treg (CD45+CD4+FoxP3+), and CD8 (CD45+CD8+)) out of total live CD45+ cells. (FIG. 6E) Representative histograms (left) and quantification (right) of FoxP3 MFI of Tregs from spleen, tdLN, and tumor. (FIGS. 6F-6H) Representative flow plots (upper) and quantification (lower) of percent (FIG. 6F) GZMB+CD4+CD45+ cells, (FIG. 6G) IFN-γ+CD8+CD45+ cells, and (FIG. 6H) IFN-γ+CD4+CD45+ cells out of total live cells from spleen, tdLN, and tumor.

Spl=spleen; tdLN=tumor-draining lymph node; TILs=tumor-infiltrating lymphocytes; GZMB=granzyme-B.

FIGS. 7A and 7B show graphical abstract schematic indicating (FIG. 7A) proposed molecular mechanism of FD03 and (FIG. 7B) working model of how FD03 treatment effects are divergent dependent on tissue-type.

FIGS. 8A, 8B, 8C, and 8D show Design and validation of FoxP3-Artichoke degradation system for high throughput screening. (FIG. 8A) Schematic of FoxP3-Artichoke plasmid and high content imaging method. (FIG. 8B) Confocal microscopy of HEK293T cells with stable FoxP3− Artichoke expression (henceforth FoxP3-Artichoke HEK293T). (FIG. 8C) Immunoblot of FoxP3− Artichoke HEK293T transiently overexpressing Myc empty vector control or Myc-STUB1. (FIG. 8D) % GFP and mCherry intensity quantified using a high content imager on a per cell basis following overnight treatment with DMSO, 5 μg/mL CHX, and 10 μM MG132 as shown in (FIG. 8A). Data is representative of n=3 technical replicates from n=1 experiment.

FIGS. 9A, 9B, 9C, and 9D show high throughput screening results using FoxP3-Artichoke stable HEK293T and top hits. (FIG. 9A) FoxP3-Artichoke HEK293T cells were treated with 5 μM compound as shown in (FIG. 8A) with DMSO, 5 μg/mL CHX, and 10 μM MG132 as controls. zScore was calculated using DMSO controls. Compounds with a mCherry zScore (left) between-0.25 to 0.5 (in green) were further analyzed by the % GFP+ zScore (right). Plotted is n=3 independent experiments with n=2 technical replicates per experiment. (FIG. 9B) Chemical structures of lead compounds. (FIG. 9C) Quantification of viability by live-dead staining and flow cytometry of MT-2 cells treated overnight with labelled compounds at varying concentrations. (FIG. 9D) Quantification of relative FoxP3 by intracellular staining and flow cytometry of MT-2 cells treated overnight with varying concentrations of FD03 or FD05 with DMSO as a control. Data from (FIGS. 9C-9D) is representative of n=4 independent experiments. All quantified data is presented as mean±s.d.

FIGS. 10A and 10B show proteomic analyses of MT-2 cells. WT MT-2 cells treated with DMSO or 40 μM FD03 or FoxP3-KO (KO) MT-2 cells were subjected to quantitative whole-proteome analyses. (FIG. 10A) Dot plot quantifying differences in identified protein abundance in DMSO control versus KO and DMSO control versus FD03-treated cells. (FIG. 10B) Quantification of FoxP3 abundance in the groups compared with Student's T-test to the DMSO control. Data is representative of n=3 for DMSO control and n=2 for FD03-treated and KO groups biological replicates from n=1 experiment.

FIG. 11 shows recovery of FoxP3 after removal of FD03. Representative Western blot and quantified data of MT-2 cells at different timepoints following FD03 treatment and wash out. MT-2 cells were treated with 20 μM FD03 for 16 hours then compound was washed out. Cells were cultured for additional 48 hours, and FoxP3 protein levels were assessed as at the indicated timepoints by Western blot. Data is normalized to the non-treated (NT) control. Data is representative of n=4 independent experiments. All quantified data is presented as mean±s.d.

FIG. 12 shows FD03 treatment does not change expression of known FoxP3-interacting deubiquitinases or E3 ligase. Immunoblot and quantification of USP7, USP22, and STUB1 from MT-2 cells treated with DMSO or 10 μM FD03.

FIGS. 13A, 13B, 13C, 13D, 13E, 13F, 13G, and 13H show analysis of murine iTregs treated with FD03. (FIGS. 13A-13D) iTregs were treated with 10 μM FD03 or DMSO control for 48 hours then assessed for different Treg markers including FoxP3 as shown in FIG. 3a. (FIG. 13E-13H) iTregs were treated with 10 μM FD03 or DMSO control for 24 hours and assessed by flow cytometry before being used in Treg suppression assay shown in FIG. 3b. (FIGS. 13A, 13E) Viability analysis by flow cytometry live-dead staining or AOPI staining with readout on the Cellometer. (FIGS. 13B, 13D) Representative flow plots of the live or CD4+ populations respectively. (FIGS. 13C, 13G) Quantification of FoxP3+ percentage out of live or CD4+ respectively. (FIGS. 13D, 13H) Quantification of FoxP3 MFI from the FoxP3+ Treg population. Data is representative of n=6 independent biological replicates from n=1 experiment. All quantified data is presented as mean±s.d.

FIGS. 14A and 14B show FD03 does not affect expression known STUB1 substrate, FoxO1. Representative histograms (left) and quantification (right) of mouse iTreg cells were treated with DMSO or 10 μM FD03 for (FIG. 14A) 24 and (FIG. 14B) 48 hours then subjected to flow cytometry to assess FoxO1 protein levels. Data is representative of n=3 biological replicates from n=1 independent experiment. All quantified data is presented as mean±s.d.

FIGS. 15A, 15B, 15C, 15D, and 15E show FoxP3 but not GITR expression recovers upon removal of FD03 in vitro. (FIG. 15A) Schematic indicating treatment schedule for results shown in (FIG. 15B). (FIG. 15B) Representative histogram (left) and quantification of FoxP3 MFI (right). (FIG. 15C) Schematic indicating treatment schedule for (FIGS. 15D-15E). (FIG. 15D) Representative histogram (left) and quantification of FoxP3 MFI (right). (FIG. 15E) Representative histogram (left) and quantification of GITR MFI (right). All data shown is representative of n=3 biological replicates from n=1 experiment. All quantified data is presented as mean±s.d.

FIGS. 16A, 16B, 16C, and 16D show FD03 does not impair Th1 and Th17 CD4+ T cell subsets. CD4+ T cells were isolated and differentiated under (FIGS. 16A-16B) Th1 or (FIGS. 16C-16D) Th17 polarizing conditions for 3-5 days before treatment for 16-20 hours by DMSO control or 10 μM FD03 and assessed by flow cytometry. (FIG. 16A) Representative flow plots (left) and quantification of TBet. (FIG. 16B) Representative flow plots (left) and quantification of IFN-γ production. (FIG. 16C) Representative flow plots (left) and quantification of RoRγt. (FIG. 16D) Representative flow plots (left) and quantification of IL-17A production. Data is representative of n=4 biological replicates and n=1 independent experiment. All quantified data is presented as mean±s.d.

FIGS. 17A, 17B, 17C, 17D, 17E, 17F, and 17G show FD03 does not impair CD8+ T cells. CD8+ T cells were isolated and activated using 2 μg/mL plate-bound anti-CD3 and anti-CD28 with 30 IU/mL IL-2 and simultaneously treated with DMSO or 10 μM FD03. All following quantification is from total CD8+ T cell population. (FIG. 17A) Representative flow plot (left) and quantification of subsets by CD62L and CD44 expression (right). (FIG. 17B) Representative histogram (left) and quantification of percentage CD25+ cells (middle) and CD25 MFI (right). (FIG. 17C) Representative histogram (left) and quantification of percentage CD69+ cells (middle) and CD69 MFI (right). (FIG. 17D) Representative histogram (left) and quantification of CD103 MFI (right). (FIG. 17E) Representative flow plot (left) and quantification of Tbet+ cells (right). (FIG. 17F) Representative flow plot (left) and quantification of granzyme B+ (GZMB+) cells (right). (FIG. 17G) Representative flow plot (left) and quantification of IFN-γ+ cells (right). Data is representative of n=3 biological replicates and n=1 independent experiment. All quantified data is presented as mean±s.d.

FIGS. 18A, 18B, and 18C show FD03 treatment in murine nTregs. (FIG. 18A) Assessment of cytotoxicity by viability staining readout by flow cytometry in Tregs treated with varying concentrations of FD03 with or without 10 ng/ml IFN-γ for 24 hours. (FIG. 18B) Representative histogram and quantification of FoxP3 MFI in murine nTregs co-treated with DMSO or varying concentrations FD03 with and without 10 ng/mL IFN-γ for 24 hours. (FIG. 18C) Representative histogram and quantification of TIGIT MFI in murine nTregs co-treated with DMSO or varying concentrations FD03 with and without 10 ng/mL IFN-γ for 48 hours. All data is representative of n=4 biological replicates and presented as mean±s.d.

FIGS. 19A, 19B, 19C, 19D, and 19E show Systemic FD03 does not affect Tregs at baseline. Healthy, WT mice were treated with vehicle control (PBS with DMSO) or 50 mg/kg FD03 systematically by either (FIGS. 19A-19B) I.P. or (FIGS. 19C-19E) I.V. administration. (FIG. 19A) Schematic depicting I.P. treatment schedule; mice were treated daily for a total of three treatments. (FIG. 19B) Relative weight change of mice to the first day of treatment (DO) via I.P. administration. (FIG. 19C) Schematic depicting I.V. treatment schedule; mice were treated every other day for a total of three times. (FIG. 19D) Relative weight change of mice to the first day of treatment (DO) via I.V. administration. Arrows in (FIGS. 19A-19D) indicate days that the treatment was given. (FIG. 19E) Representative flow plots and quantification of percentage of FoxP3+ cells out of total splenic CD4+ T cells and FoxP3 MFI of FoxP3+ cells in mice treated as the schematic in (FIG. 19C) depicts. All data is representative of n=3 biological replicates from n=1 experiment, and all quantified data is presented as mean±s.d.

FIGS. 20A, 20B, 20C, 20D, 20E, and 20F show FD03 treatment does not impair tumor growth in a tumor-intrinsic manner. (FIG. 20A) B16 or (FIG. 20B) MC38 cells were plated, allowed to adhere overnight, then treated with DMSO control or 10 μM FD03. Media was refreshed with new treatment every other day, and cells were collected daily to obtain cell number. Cells were counted by staining with AOPI and counted on the Cellometer. Data is representative of n=4 technical replicates from n=1 independent experiment. (FIGS. 20C-20D) B16-F10 melanoma cells were implanted subcutaneously onto the flank of Rag1−/− mice. Mice were treated daily with PBS vehicle control or 5 mg/kg FD03 for a total of 5 treatments starting when the tumors averaged 100 mm³. (FIG. 20C) Quantification of tumor volume over time. (FIG. 20D) Quantification of tumor weight at time of euthanasia. Data from (FIGS. 20C-20D) is representative of n=5 biological replicates from n=1 independent experiment. (FIGS. 20E-20F) MC38 colon carcinoma cells were implanted subcutaneously onto the flank of Rag1−/− mice. Mice were treated daily with PBS vehicle control or 5 mg/kg FD03 for a total of 6 treatments starting when the tumors averaged 100 mm³. (FIG. 20E) Quantification of tumor volume over time. (FIG. 20F) Quantification of tumor weight at time of euthanasia. Data from (FIGS. 20E-20F) is representative of n=3 biological replicates from n=2 independent experiments. All quantified data is presented as mean±s.d.

FIGS. 21A, 21B, 21C, 21D, 21E, 21F, 21G, 21H, 21I, and 21J show immunoprofiling of EG7 lymphoma and MC38 colon carcinoma treated with FD03 peritumorally. (FIGS. 21A-21E) TIL analyses from EG7 lymphoma shown in FIG. 5c-d. (FIGS. 21F-21J) TIL analyses from MC38 colon carcinoma shown in FIG. 5e-f. (FIGS. 21A, 21F) Representative flow plots (left) and quantification of Tconv (CD45+CD4+FoxP3−), Treg (CD45+CD4+FoxP3+), and CD8 (CD45+CD8+) out of total live CD45+ cells. (FIGS. 21B, 21G) Representative flow plots (upper) and quantification (lower) of IFN-γ+CD45+CD4+ cells out of total CD45+CD4+ cells. (FIGS. 21C, 21H) Representative flow plots (left) of total CD45+CD4+ cells gated on FoxP3+ and quantification (right) of FoxP3 MFI relative to vehicle control in Tregs. (SFIGS. 21D, 21I) Representative flow plots (upper) and quantification (lower) of IFN-γ+CD45+CD4+ cells out of total CD45+CD8+ cells. (FIGS. 21E, 21J) Representative flow plots (upper) and quantification (lower) of GZMB+ CD8 cells out of total CD45+CD8+. Data from (FIGS. 21A-21E) is representative of n=8 biological replicates from n=2 independent experiments; data from (FIG. 21F-21J) is representative of n=5-6 biological replicates from n=1 independent experiment. All quantified data is presented as mean±s.d.

FIG. 22 shows peritumoral FD03 treatment does not induce shifts in immune cell populations. TILs from isotype-treated tumors from FIG. 5p-q were analyzed by flow cytometry. Cell populations were defined as following: T cells=CD3+B220−; B cells=CD3−B220+; Macrophages (MΦ)=CD3−B220−Ly6G−/loF4/80+; Neutrophil/MDSC=CD3−B220−CD11b+Ly6Ghi; DC/NK/Mono/Other=CD3−B220−Ly6G−/loF4/80−.

FIG. 23 shows systemic FD03 treatment does not induce acute toxicity. Mice from FIG. 6 were analyzed for acute toxicity following systematic treatment with FD03 via I.P. administration. Relative weight change of mice to the first day of treatment (DO); arrows indicate days treatment was given. Data from FIG. 23 is representative of n=7 mice from n=1 experiment.

FIGS. 24A, 24B, 24C, 24D, and 24E show immunoprofiling of spleen and tumor-draining lymph node of tumor-bearing mice treated systemically with FD03. Analysis from spleens and tdLNs from tumor-bearing mice shown in FIG. 6. (FIGS. 24A-24B) Representative flow plot (left) and quantification of immune cell subsets (T=T cells (CD3+B220−); B=B cells (CD3−B220+); Non-T/B=Non-T/B cells (CD3−B220−)) from (FIG. 24A) spleen or (FIG. 24B) tdLN. (FIGS. 24C-24D) Representative flow plot (left) and quantification of CD3+ T cell subsets (Tconv (CD4+FoxP3−); Treg (CD4+FoxP3+); CD8+ (CD8+)) from (FIG. 24C) spleen and (FIG. 24D) tdLN. (FIG. 24E) Analysis of myeloid cells from spleen. CD3−B220− splenic cells were defined as published in Faget, et al (2017).

### DETAILED DESCRIPTION OF THE INVENTION

Disclosed herein are methods of treating a disease or disorder associated with forkhead box P3 (FoxP3) expression in a subject in need thereof, methods of inducing proteolytic degradation of forkhead box P3 (FoxP3) in a cell, and small molecule FoxP3 degraders.

Methods of Treating a Disease or Disorder Associated with FoxP3 Expression

FoxP3 is the lineage-defining transcription factor for T regulatory cells (Tregs) which are known to negatively regulate anti-tumor immune responses. It was discovered that several compounds reduce FoxP3 expression in a cell culture model.

Accordingly, methods of treating a disease or disorder associated with FoxP3 expression in a subject in need thereof are provided. In some embodiments, the methods comprise administering a therapeutically effective amount of a small molecule FoxP3 degrader. In some instances in the present disclosure, the small molecule FoxP3 degrader may be referred to as the compound. In some instances, the small molecule FoxP3 degrader has a formula selected from:

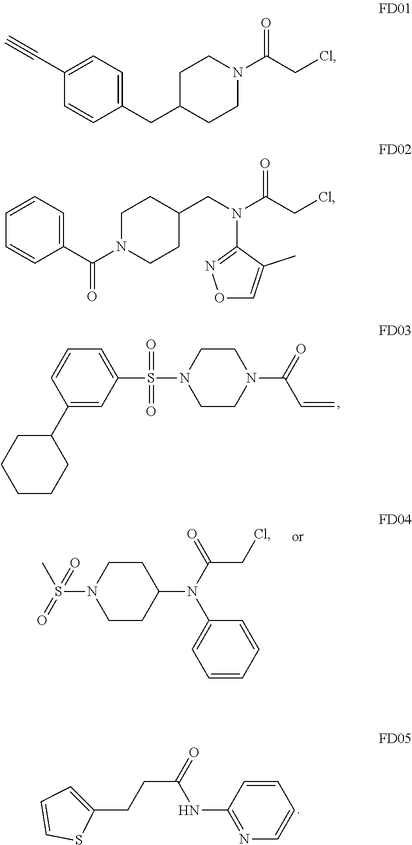 [SMILES: C#Cc1ccc(CC2CCN(C(=O)CCl)CC2)cc1 ; Cc1conc1N(CC1CCN(C(=O)c2ccccc2)CC1)C(=O)CCl ; C=CC(=O)N1CCN(S(=O)(=O)c2cccc(C3CCCCC3)c2)CC1 ; CS(=O)(=O)N1CCC(N(C(=O)CCl)c2ccccc2)CC1 ; O=C(CCc1cccs1)Nc1ccccn1]

The compound may be FD03:

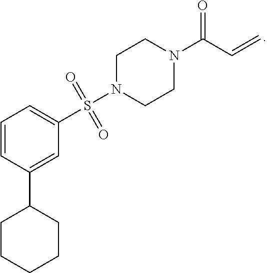 [SMILES: C=CC(=O)N1CCN(S(=O)(=O)c2cccc(C3CCCCC3)c2)CC1]

The human reference amino acid sequence for FoxP3 is included in this disclosure as SEQ ID NO: 4.

Diseases or disorders associated with FoxP3 expression may comprise any disease or disorder characterized by either increased FoxP3 expression or characterized by the detrimental involvement of Tregs, which express FoxP3. For example, diseases or disorders associated with FoxP3 expression may comprise cell proliferative diseases or disorders, e.g., cancer, where Tregs are known to support an immunosuppressive tumor immune microenvironment (TIME). Diseases or disorders associated with FoxP3 expression may comprise chronic viral infections, e.g., human immunodeficiency virus (HIV) infection, hepatitis B virus (HBV) infection, hepatitis C virus (HCV) infection, or human papillomavirus (HPV) infection, especially, the chronic phase of any viral infection, e.g., at least 7, 8, 9, 10, 11, 12, 13, 14, or more days after infection.

The FoxP3 degrader may be administered in any suitable dose as determined by a physician. The suitable dose may be, e.g., about 1 mg/kg to about 100 mg/kg, about 30 mg/kg to about 70 mg/kg, about 40 mg/kg to about 60 mg/kg, or any subrange or value in the foregoing ranges, e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg.

In one non-limiting example, the inventors analyzed clinical outcomes in the Cancer Genome Atlas using FoxP3 as a predictive biomarker and found that high FoxP3 expression is associated with poor clinical outcomes in 13 types of cancer: adrenocortical carcinoma, cervical squamous cell carcinoma, endocervical adenocarcinoma, glioblastoma multiforme, head and neck squamous cell carcinoma, renal clear cell carcinoma, renal papillary cell carcinoma, acute myeloid leukemia, lung adenocarcinoma, ovarian serous cystadenocarcinoma, rectal adenocarcinoma, cutaneous melanoma, thymoma, and uveal melanoma. (see Example 2 and Table 1).

Thus, the cancer may comprise adrenocortical carcinoma, cervical squamous cell carcinoma, endocervical adenocarcinoma, glioblastoma multiforme, head and neck squamous cell carcinoma, renal clear cell carcinoma, renal papillary cell carcinoma, acute myeloid leukemia, lung adenocarcinoma, ovarian serous cystadenocarcinoma, rectal adenocarcinoma, cutaneous melanoma, thymoma, or uveal melanoma. Even in cancer types where FoxP3 expression levels are not linked to clinical outcomes, its degradation could still potentially enhance antitumor immune responses against these cancers because FoxP3 degradation can result in enhancement of systemic immune response. Therefore, a FoxP3 degrader holds therapeutic potential to treat all types of human tumors.

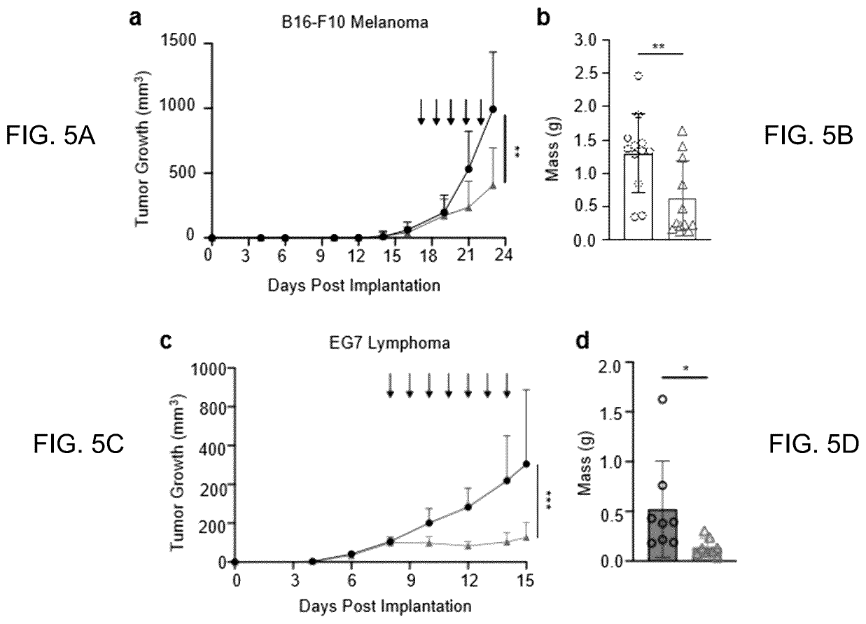

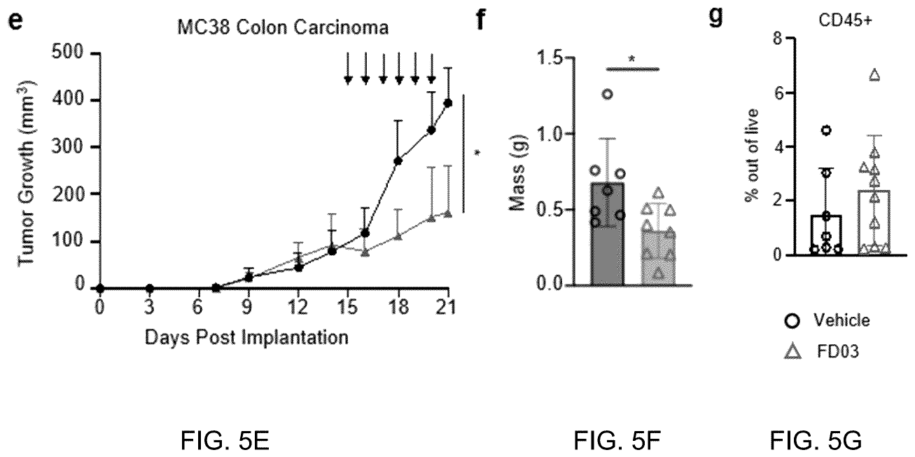

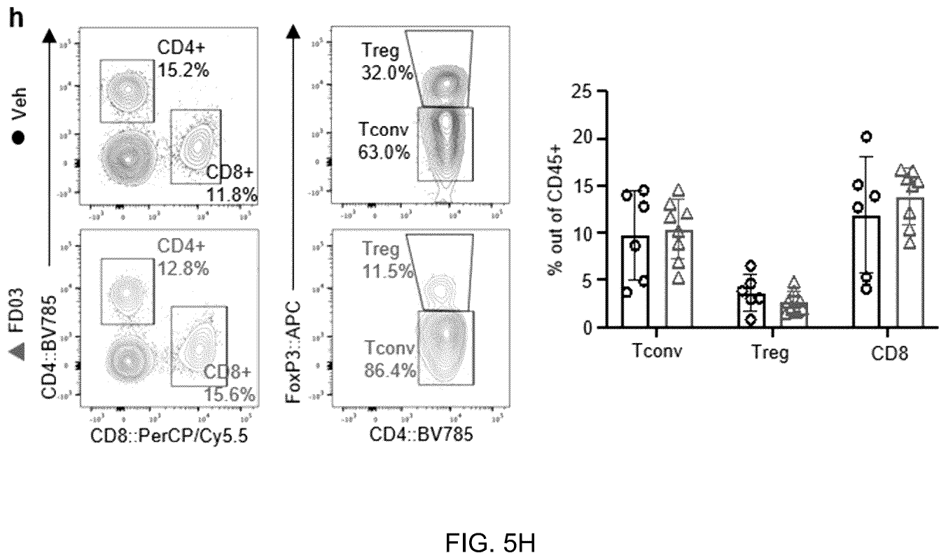

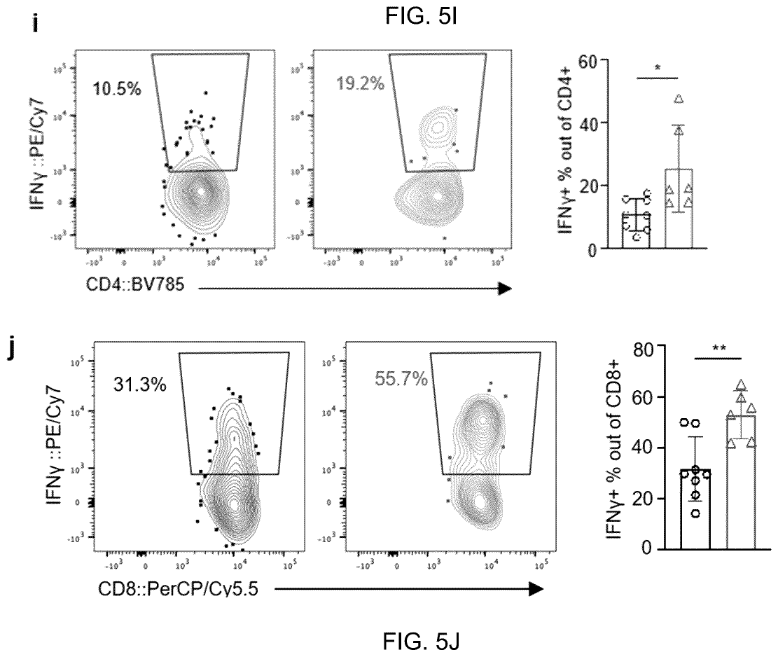

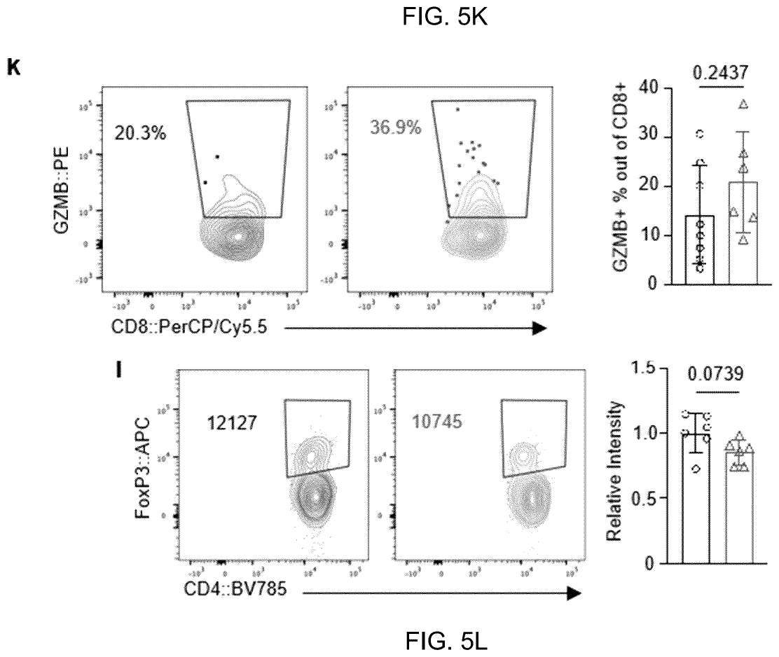

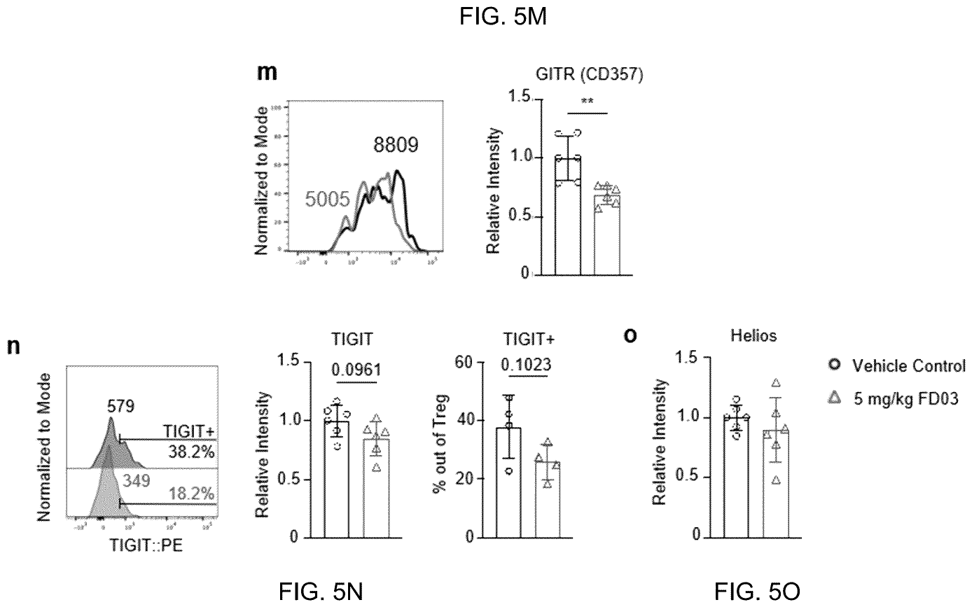

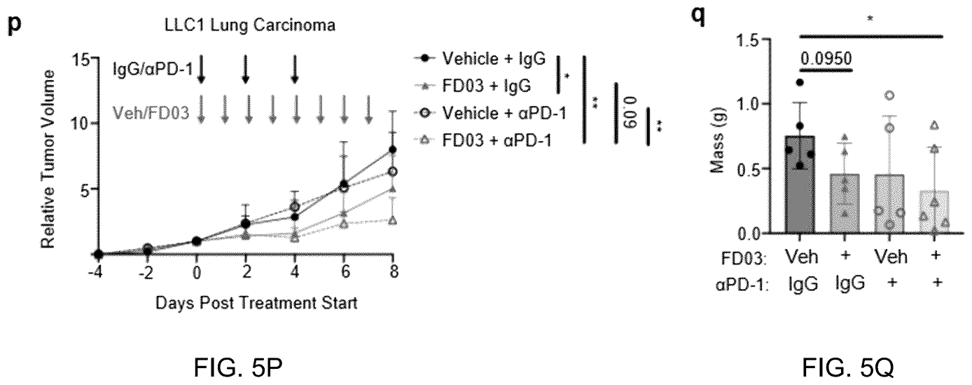

The inventors discovered that the disclosed Foxp3 degraders are effective in reducing volume of B16 tumors (melanoma model), LLC1 lung carcinoma (lung carcinoma model) EG7 (lymphoma model), and MC38 tumors (colorectal cancer model) in mice (FIG. 5). Thus, without limitation, the cancer may comprise melanoma, lymphoma, lung carcinoma, or colorectal cancer.

Combination therapies are also provided, wherein the disclosed methods further comprise administering an additional therapy, e.g., a chemotherapy, a targeted therapy, a surgical therapy, a radiation therapy, an immunotherapy, or a hormone therapy.

The additional therapy may comprise an immunotherapy. Suitably, the immunotherapy may comprise an immune checkpoint blocker (ICB). Immune checkpoint blocker refers to a compound that interferes with at least one “immune checkpoint molecule,” e.g., PD-1, CTLA-4, LAG3, from being ligated and/or transducing an intracellular signal, e.g., in a T cell. The ICB may be an immune checkpoint inhibitor (ICI). Exemplary ICIs include, but are not limited to, therapies that inhibitor PD-1 and PD-L1 interactions, e.g., PD-1 inhibitors (e.g., Pembrolizumab, Nivolumab, Cemiplimab), PD-L1 inhibitors (e.g., Atezolizumab, Avelumab, Durvalumab), CTLA-4 inhibitors (e.g., Ipilimumab, Tremelimumab), LAG-3 inhibitors (e.g., Relatlimab).

The immunotherapy may comprise an inflammatory agent. The inflammatory agent may comprise, e.g., a cytokine or an immune-stimulating agent. The cytokine may be, e.g., interleukin 1b (IL-1b) or IFN-γ. The immune-stimulating agent may comprise, e.g., lipopolysaccharide (LPS). In some instances the inflammatory agent modulates expression of STUB1 in the Treg, such as inducing expression of STUB1.

As used herein, a “therapeutically effective amount” or an “effective amount” refers to the amount of an agent to cause a desired effect in a subject. In one example, a therapeutically effective amount is an amount that is effective in reducing at least one sign or symptom of a disease or disorder associated with FoxP3 expression in a subject. In the case of a cancer with one or more solid tumors, a therapeutically effective amount is sufficient to cause, for example, at least one of a reduction in tumor volume, a reduction in tumor number, a reduction in tumor markers in the blood, or a reduction in tumor cell-free DNA (cfDNA). In the case of liquid tumors, a therapeutically effective amount is sufficient to, for example, cause at least one of a reduction in tumor burden, a reduction in tumor markers in the blood, a reduction in tumor cell-free DNA (cfDNA). In the case of cell proliferative diseases and disorders, a therapeutically effective amount is sufficient to, for example, cause a reduction in number or volume of aberrantly proliferating cells.

As used herein, a “subject in need thereof” refers to a subject in need of treatment by the disclosed methods, e.g., a subject in need of treatment for a disease or disorder associated with FoxP3 expression, as defined herein.

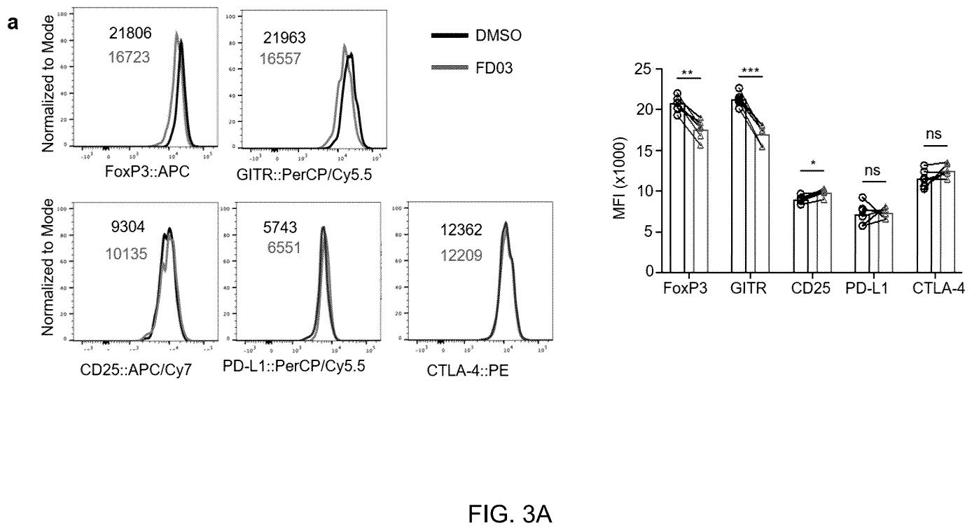

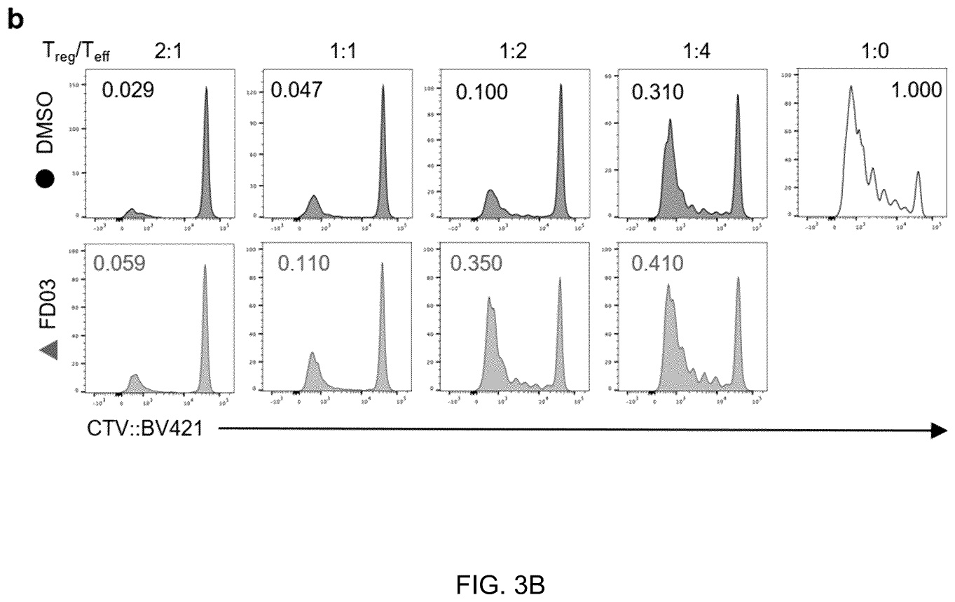

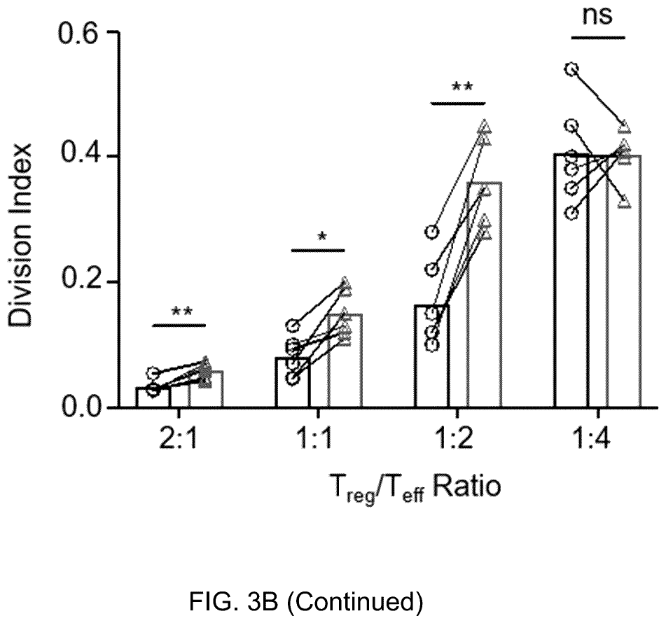

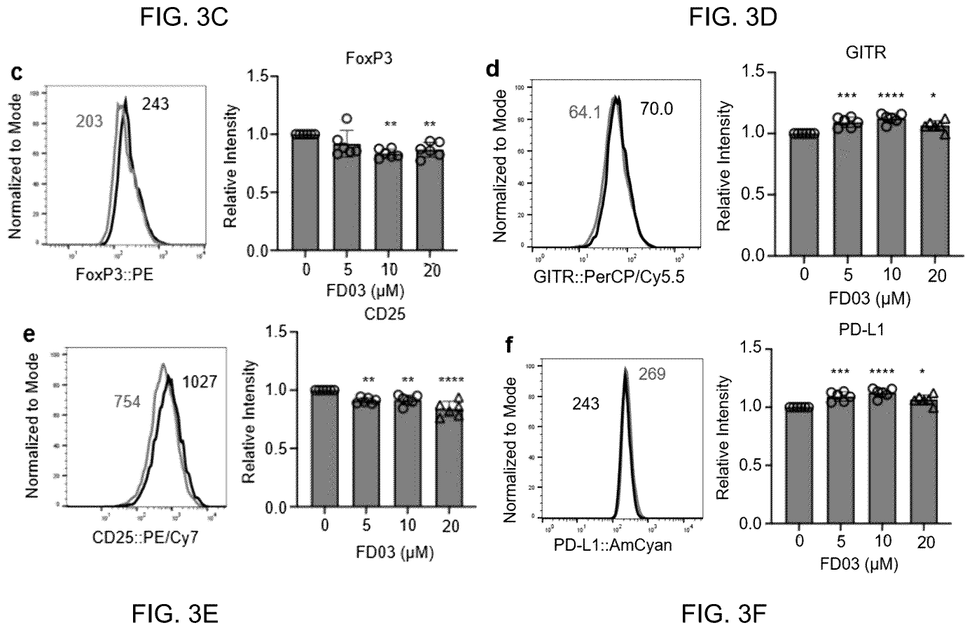

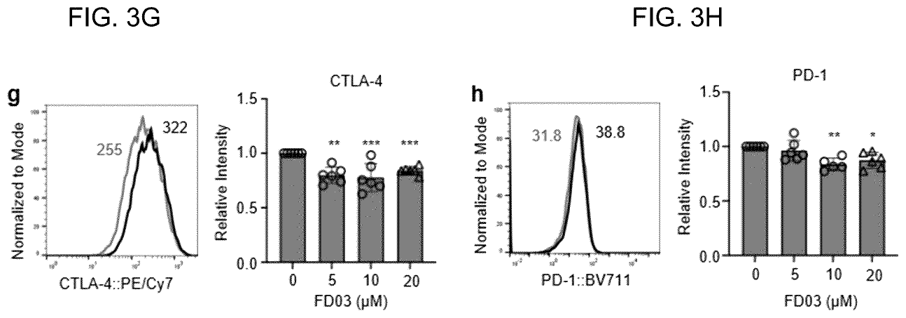

The inventors demonstrated that the disclosed compounds decrease glucocorticoid-induced TNFR-related protein (GITR) expression (see, e.g., FIG. 3B), which is indicative of reduced Treg effector functions. Accordingly, in some embodiments, the methods decrease GITR expression, e.g., in Tregs which may be measured by methods known in the art, e.g., flow cytometry.

The compound may be administered by any suitable route, e.g., orally, intravenously, intraperitoneally, intrathecally, intramuscularly, peritumorally, or intratumorally. In some embodiments, the compounds are administered intratumorally, e.g., as performed in Example 1.

### Advantages of the Disclosed Technology

Tregs are critical for tumor immune evasion and their accumulation is responsible for the resistance to the current antitumor immunotherapies.

Targeting the Treg population or Treg suppressive function has been shown to be efficacious in improving immune checkpoint blockade outcomes, but this can only be achieved using genetically modified experimental mouse models.

The current approaches to target Tregs are non-specific.

This is a small molecule and can target and degrade endogenous FoxP3; other published molecules or methodologies can only target a tagged FoxP3 (i.e. GFP-FoxP3) or use peptide-ligands which are more difficult and expansive to manufacture.

The antitumor immune Checkpoint blockade (ICB) therapy has transformed oncology by harnessing the immune system to combat tumors. However, many patients exhibit either primary resistance (lack of initial response) or acquired resistance (loss of response after an initial benefit). Treg accumulation is known as a major critical factor responsible for the current antitumor immunotherapeutic resistance. There continues to be an unmet need in cancer therapeutics given the low complete response rate of immune checkpoint blockade in patients. In the past years, there has been increased interest and understanding that modulating the Treg immune response can play a pivotal role in cancer outcomes, but largely unsuccessful. Our small molecule can diminish Treg immunosuppression function to boost anti-tumor immunity presumably in patients with any type of cancers. In addition to cancer patients, the immune suppressive Tregs are responsible for the impaired anti-microbial infections in patients with many chronic infectious disease, such as hepatitis B virus (HBV), hepatitis C virus (HCV), and human papillomavirus (HPV). Targeted therapy by FD03 may improve the anti-microbial immunity to clear the infections in these patients.

### Methods of Inducing Degradation of FoxP3 in a Cell

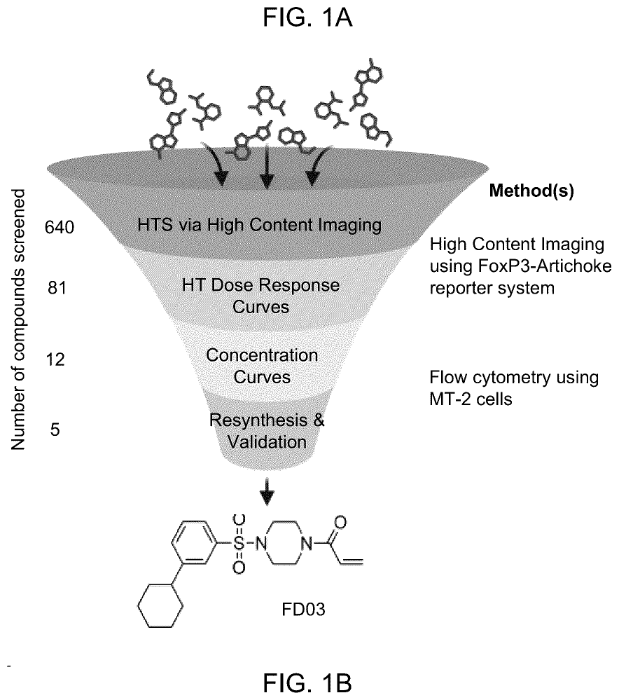

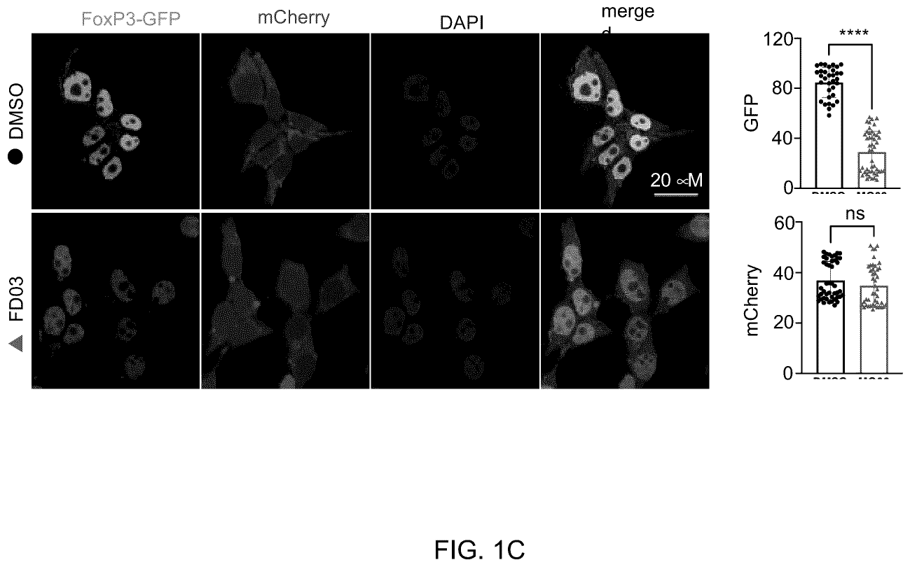

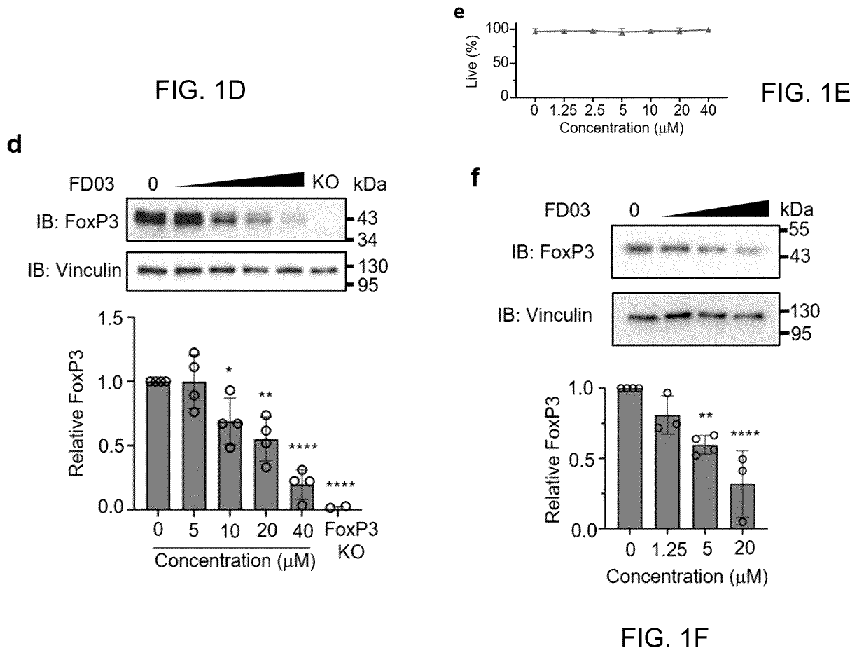

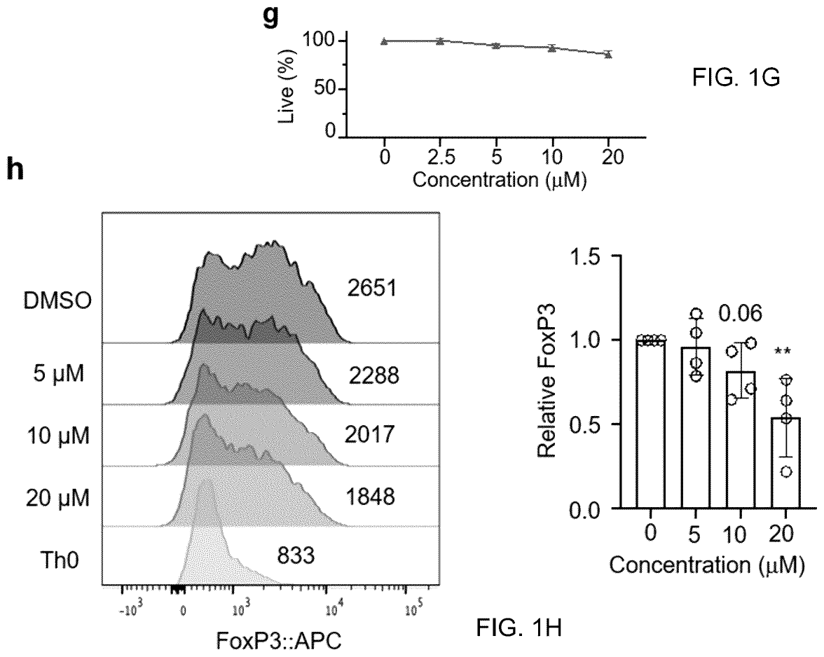

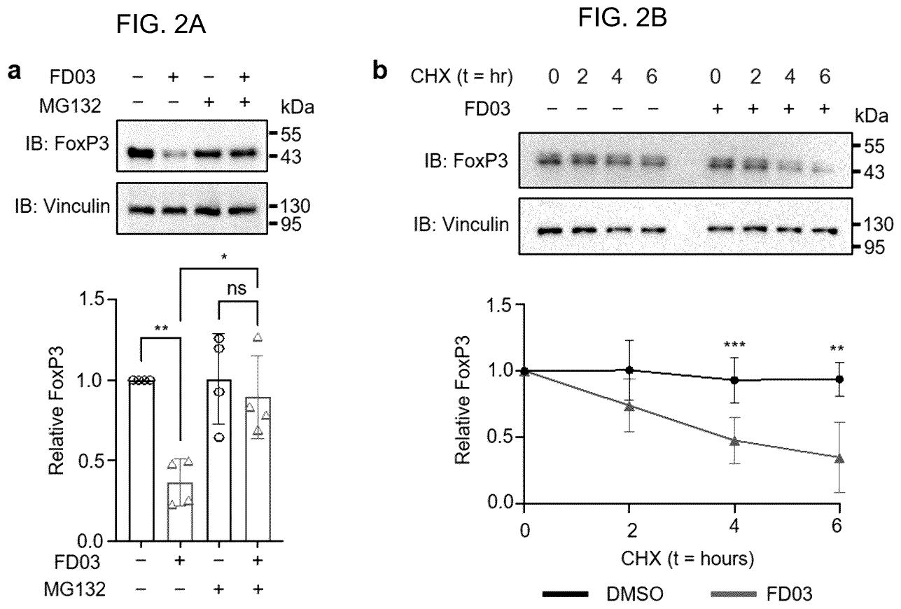

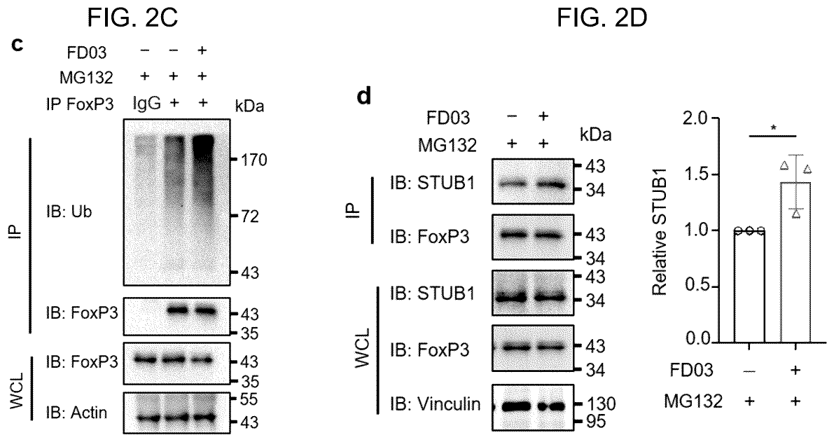

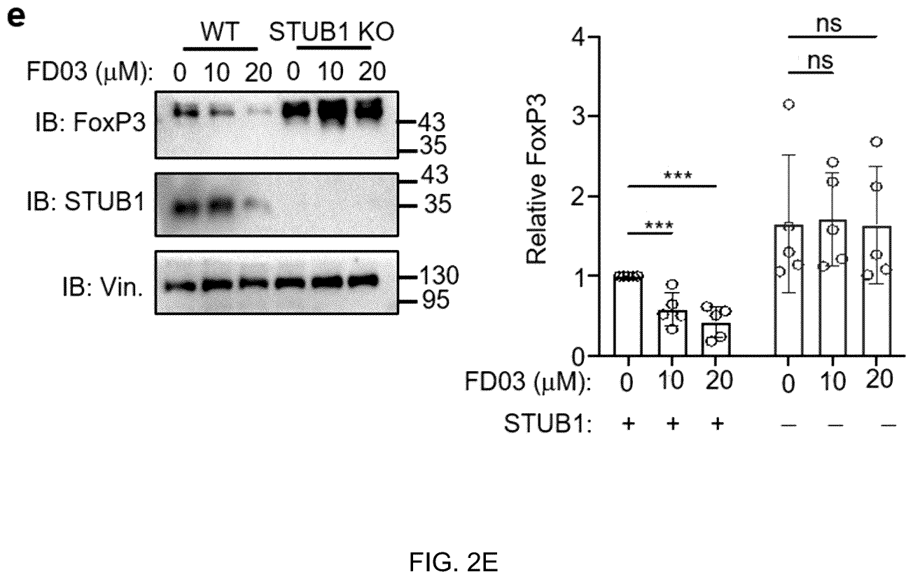

The inventors discovered compounds that are capable of reducing FoxP3 expression in a cell culture model. Without wishing to be limited by any theory or mechanism, the compounds are believed to induce degradation of FoxP3 (see, e.g., FIGS. 1C, 1D, 1F, and 2B) by the endogenous proteolytic machinery in the cell (see, e.g., FIGS. 2A and 2B). Accordingly, in an aspect of the current disclosure, methods of inducing proteolytic degradation of FoxP3 in a cell are provided. In some embodiments, the methods comprise contacting the cell with a compound having a formula selected from:

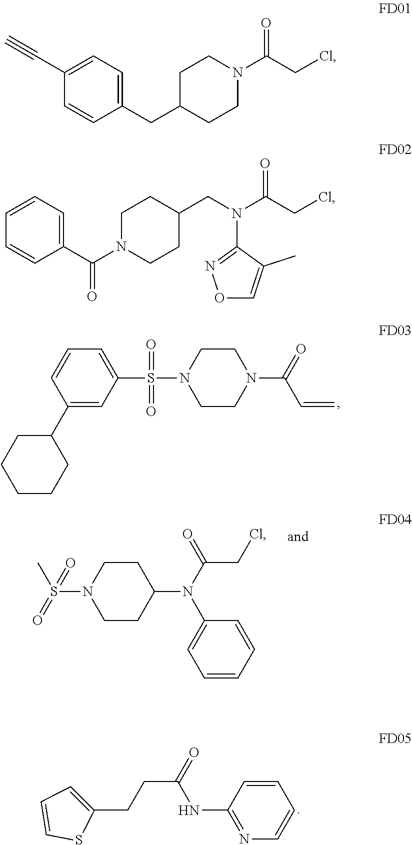 [SMILES: C#Cc1ccc(CC2CCN(C(=O)CCl)CC2)cc1 ; Cc1conc1N(CC1CCN(C(=O)c2ccccc2)CC1)C(=O)CCl ; C=CC(=O)N1CCN(S(=O)(=O)c2cccc(C3CCCCC3)c2)CC1 ; CS(=O)(=O)N1CCC(N(C(=O)CCl)c2ccccc2)CC1 ; O=C(CCc1cccs1)Nc1ccccn1]

The compound may be FD03:

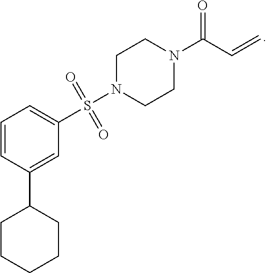 [SMILES: C=CC(=O)N1CCN(S(=O)(=O)c2cccc(C3CCCCC3)c2)CC1]

FoxP3 is the lineage defining transcription factor for Tregs; therefore, in some embodiments, the cell is a Treg. The Treg may be either a CD4 Treg or a CD8 Treg. The cells may be cells from a human, dog, cat, horse, donkey, sheep, etc.

The cells may be contacted with an “effective amount” of the compound which refers to an amount sufficient to induce degradation of FoxP3. Degradation of FoxP3 can be measured by the skilled artisan in a variety of ways, including, but not limited to, Western blotting or mass spectroscopy. The amino acid sequence of human FoxP3 is provided as SEQ ID NO: 4. The sequence of FoxP3 may be used by a skilled artisan to either detect FoxP3, e.g., by mass spectroscopy. Antibodies specific for FoxP3 are known in the art and discussed, e.g., in Example 1. Such antibodies could be used to detect FoxP3 in a sample.

In some embodiments, the cell is contacted with the small molecule FoxP3 degrader in the presence of interleukin 1β (IL-1β), lipopolysaccharide (LPS), or IFN-γ.

### Pharmaceutical Compositions

In an aspect of the current disclosure, pharmaceutical compositions are provided and comprise a small molecule FoxP3 degrader. The small molecule FoxP3 degrader may have a formula selected from:

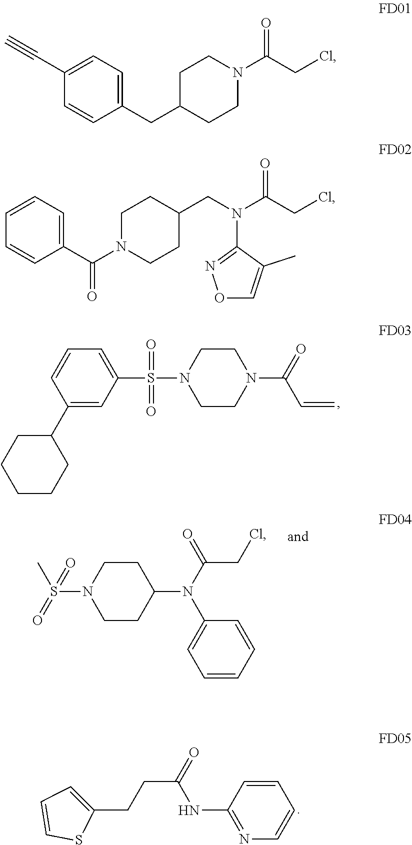 [SMILES: C#Cc1ccc(CC2CCN(C(=O)CCl)CC2)cc1 ; Cc1conc1N(CC1CCN(C(=O)c2ccccc2)CC1)C(=O)CCl ; C=CC(=O)N1CCN(S(=O)(=O)c2cccc(C3CCCCC3)c2)CC1 ; CS(=O)(=O)N1CCC(N(C(=O)CCl)c2ccccc2)CC1 ; O=C(CCc1cccs1)Nc1ccccn1]

The compound may be FD03:

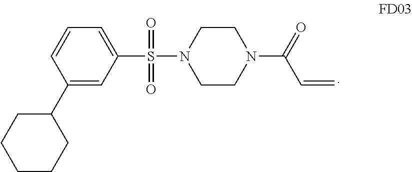 [SMILES: C=CC(=O)N1CCN(S(=O)(=O)c2cccc(C3CCCCC3)c2)CC1]

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used.

In some embodiments, the pharmaceutical compositions are formulated for administration by one or more of the following routes: orally, intravenously, intraperitoneally, intrathecally, intramuscularly, or intratumorally.

In some embodiments, the pharmaceutical compositions further comprise another agent, e.g., a chemotherapy, an immunotherapy, or a hormone therapy.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

In some embodiments, the compounds disclosed herein may be formulated as pharmaceutical compositions that include: (a) a therapeutically effective amount of one or more compounds as disclosed herein; and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to about 1000 mg/kg body weight (preferably about 0.5 to about 500 mg/kg body weight, more preferably about 50 to about 100 mg/kg body weight). In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 nM, 0.005 nM, 0.01 nM, 0.5 nM, 0.1 nM, 1.0 nM, 10 nM, and 100 nM (e.g., 0.1 nM-1.0 nM).

It is understood by those skilled in the art that dosage amount will vary with the activity of a particular inhibitor compound, disease state, route of administration, duration of treatment, and like factors well-known in the medical and pharmaceutical arts. In general, a suitable dose will be an amount which is the lowest dose effective to produce a therapeutic or prophylactic effect. If desired, an effective dose of such a compound, pharmaceutically acceptable salt thereof, or related composition may be administered in two or more sub-doses, administered separately over an appropriate period of time.

The compounds for use according to the methods of disclosed herein may be administered as a single compound or a combination of compounds. For example, a compound that inhibits FoxP3 activity or expression may be administered as a single compound or in combination with another compound that inhibits FoxP3 activity or expression, or that has a different pharmacological activity. In some embodiments, one or more additional therapeutic agents may be administered with the disclosed compounds or with pharmaceutical compositions comprising the disclosed compounds, where the additional therapeutic agent is administered prior to, concurrently with, or after administering the disclosed compounds or the pharmaceutical compositions comprising the disclosed compounds. In some embodiments, the disclosed pharmaceutical composition is formulated to comprise the disclosed compounds and further to comprise one or more additional therapeutic agents, for example, one or more additional therapeutic agents for treating diseases and disorders.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-. 1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein may not be critical to the activity of the compound, so long as the salt as a whole is pharmacologically acceptable and as long as the counterion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

The pharmaceutical composition may include the compound in a range of about 0.1 to 2000 mg. In some embodiments, the pharmaceutical composition may include the compound in a range of from about 0.5 to 500 mg. In some embodiments, the pharmaceutical composition may include the compound in a range of from about 1 to 100 mg. The pharmaceutical composition may be administered to provide the compound at a daily dose of about 0.1 to about 1000 mg/kg body weight. In some embodiments, the pharmaceutical composition may be administered to provide the compound at a daily dose of about 0.5 to about 500 mg/kg body weight. In some embodiments, the pharmaceutical composition may be administered to provide the compound at a daily dose of about 50 to about 100 mg/kg body weight. A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment. In some embodiments, after the pharmaceutical composition is administered to a subject (e.g., after about 1, 2, 3, 4, 5, or 6 hours post-administration), the concentration of the compound at the site of action may be within a concentration range bounded by end-points selected from 0.001 nM, 0.005 nM, 0.01 nM, 0.5 nM, 0.1 nM, 1.0 nM, 10 nM, and 100 nM (e.g., 0.1 nM-1.0 nM).

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Filling agents may include lactose monohydrate, lactose anhydrous, and various starches; examples of binding agents are various celluloses and crosslinked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil®200, talc, stearic acid, magnesium stearate, calcium stearate, and silica gel. Examples of sweeteners may include any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents may include pharmaceutically acceptable inert fillers, such as microcrystalline cellulose, lactose, dibasic calcium phosphate, saccharides, and mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the pharmaceutical compositions are in some embodiments applied as a topical ointment or cream. When formulated in an ointment, the compound may be employed with either a paraffinic or a water-miscible ointment base. Alternatively, the compound may be formulated in a cream with an oil-in-water cream base or a water-in-oil base. Pharmaceutical compositions adapted for topical administration to the eye include eye drops where the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration where the carrier is a solid include a coarse powder having a particle size (e.g., in the range 20 to 500 microns) which is administered in the manner in which snuff is taken (i.e., by rapid inhalation through the nasal passage from a container of the powder held close up to the nose). Suitable formulations where the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

Methods of preparing pharmaceutical formulations or compositions include the step of bringing a disclosed compound into association with a carrier and, optionally, one or more additional adjuvants or ingredients. For example, standard pharmaceutical formulation techniques can be employed, such as those described in Remington's Pharmaceutical Sciences, Mack Publishing Company, Easton, PA.

Regardless of composition or formulation, those skilled in the art will recognize various avenues for medicament administration, together with corresponding factors and parameters to be considered in rendering such a medicament suitable for administration.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

As used herein, “salt” refers to acid addition salts and basic addition salts. It may also refer to those salts that may be prepared in situ during the final isolation and purification of the compounds of the invention.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid, and citric acid.

Basic addition salts may be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

Compounds described herein may exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1—Development of a FoxP3-specific Small Molecule Degrader

Regulatory T cell (Treg) infiltration and accumulation within solid tumors leads to tumor immune evasion. Thus, immunotherapeutic strategies aimed at reducing Treg abundance and/or suppressive function hold significant promise for cancer treatment. However, over the past two decades, efforts to modulating Treg cells to overcome immune checkpoint blockade resistance have largely relied on non-specific molecular or cellular approaches. Forkhead box protein 3 (FoxP3), the master transcription factor of Treg cells, is essential for Treg differentiation, lineage stability and immunosuppressive function. Due to its nature as a transcription factor, FoxP3 has been notoriously difficult to pharmacologically target. Here, we report the first FoxP3-specific small molecule degrader, termed FD03, which can promote proteasomal degradation of FoxP3 and partially reduce Treg suppressive function. We identified FD03 through a targeted screen of a library of electrophilic small molecule compounds. We show FD03 treatment in Treg cells in vitro diminishes their suppressive capabilities, while in vivo administration in tumor-bearing mice significantly reduces tumor burden. Mechanistically, FD03 degrades FoxP3 in part through facilitating FoxP3 interaction with its E3 ligase, STUB1 (CHIP), to promote FoxP3 ubiquitination-mediated proteasomal degradation. Most excitingly, FD03 appears to require sufficient expression of STUB1 to induce FoxP3 reduction, and we observe FoxP3 decrease in only tumoral but not splenic Tregs upon systemic FD03 treatment in tumor-bearing mice. All in all, this study identifies FD03 as a promising lead compound and offers proof-of-concept for the development of small molecule degraders that directly target FoxP3 to decrease Treg immunosuppression and boost anti-tumor immunity.

### INTRODUCTION

T regulatory cells (Treg cells) are a subset of CD4+ T cells which are required to maintain peripheral immune tolerance and prevent autoimmunity through their immunosuppressive functions¹-3. However, in many different types of cancer, Treg cells suppress the anti-cancer immune response in multifaceted methods including but not limited to competition for key cytokines such as IL-2, expression of inhibitory receptors such as CTLA-4 and PD-L1, and release of inhibitory cytokines such as IL-10^{1,2,4-6}. Moreover, Treg cells play a major role in the lack of complete response or disease regression after immune checkpoint blockade due to their multiple suppressive mechanisms or reinvigoration of suppressive Treg cells alongside cytotoxic T cells^{4,5,7,8} As a result, Treg cells have come under scrutiny as a major target for tumor therapeutics. Treg depletion has been shown to be beneficial to animals bearing tumors. However, selectively targeting Treg cells remains technically challenging due to their phenotypic and functional similarities with tumor-killing cytotoxic T cells. For example, while strategies involving CD25 monoclonal antibodies can efficiently deplete intratumoral Treg cells, these approaches lack Treg specificity and often result in only modest efficacy. This is likely due to concurrent effects on activated effector T cells, all of which also express CD25^{2,7-13}. Consequently, there is a critical need for therapies that selectively inhibit Treg function without compromising conventional T cell-mediated antitumor immunity.

The transcription factor Forkhead box protein 3 (FoxP3) has been well-studied as the main driver and lineage delineating factor of Treg cells^{1,14,15}. Maintenance of FoxP3 is crucial for the stability and suppressive functions of Treg cells^{1,14-17}. Notably, FoxP3 is expressed predominantly in Treg cells, and FoxP3 expression is increased significantly in tumor-infiltrating Treg cells^{1,18} Altogether, FoxP3 is an extraordinarily appealing target for modulating Treg-mediated immunosuppression in tumors due to its necessity and specificity in Treg cells. Transcription factors have been notoriously difficult to inhibit with small molecules. Other pharmacological strategies have been used to decrease or inhibit FoxP3 including an anti-sense oligonucleotide (AZD8701) and a peptide-based inhibitor (P60). As targeted protein degradation (TPD) drug strategies emerged, one group utilized proteolysis-targeting chimeric (PROTAC) drug design strategies and developed a peptide-PROTAC molecule (P60-promalidomide) to target Tregs. Moreover, these pharmaceuticals have poor in vivo efficacy or do not differentiate between intratumoral Tregs and Tregs in healthy tissue. In short, no small molecules targeting FoxP3 or pharmaceuticals specific to tumoral Tregs have been reported to date.

The TPD strategy harnesses the ubiquitin-proteasome system (UPS) for degradation of proteins of interest¹⁹-23. Our lab has previously shown that modulation of FoxP3 by chemical inhibition of endogenous deubiquitinase USP22 proves to be an effective therapeutic in preclinical mouse models^{16,18} However, USP22 is ubiquitously expressed, and the ability to directly target FoxP3 via a small molecule degrader is a more promising and precise therapeutic. Herein, we demonstrate the feasibility of drugging FoxP3 using a small molecule employing the TPD strategy to reliably reduce Treg-mediated immunosuppression.

### RESULTS

### Identification of FD03 by High Throughput Screening.

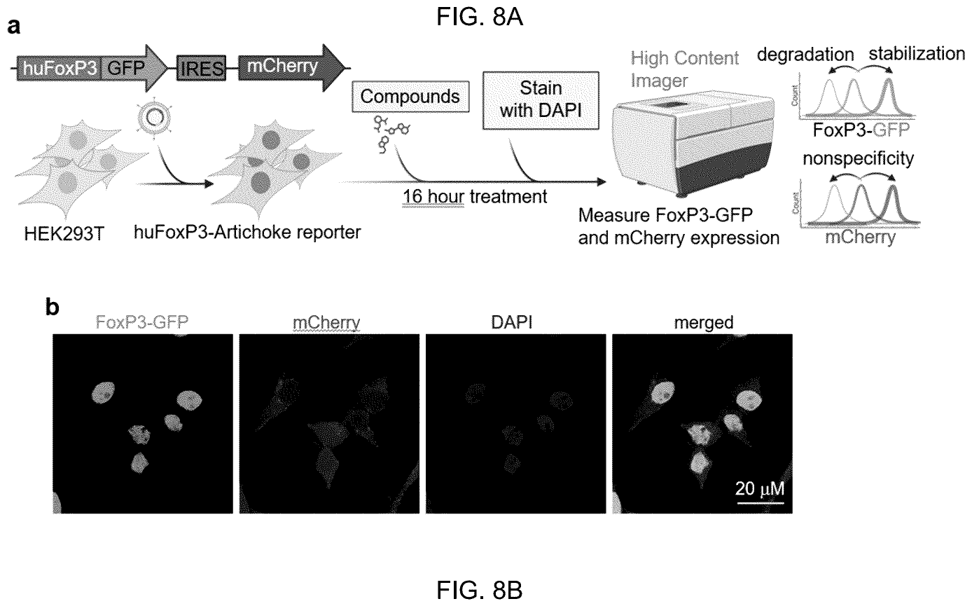

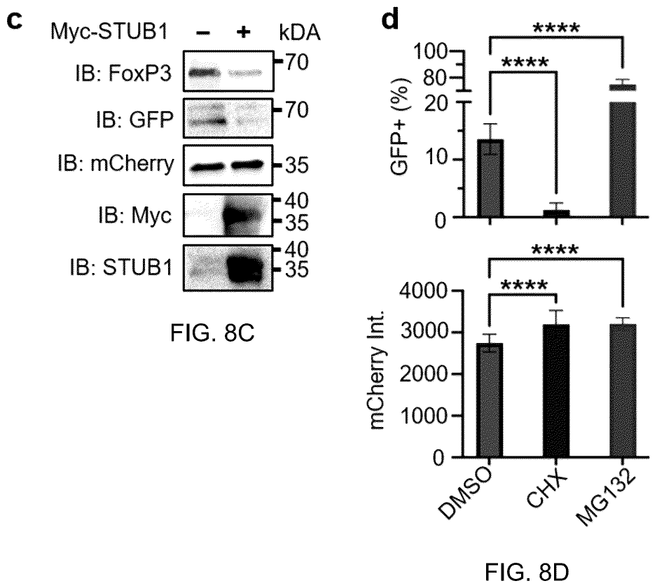

We sought to identify small molecules which can induce degradation of FoxP3 protein by high throughput screening of a targeted small molecule library. To enable a high-throughput and specific assessment of small molecules for specific FoxP3 degradation, we employed the Artichoke degradation reporter system and generated a HEK293T cell line stably expressing Artichoke plasmid with FoxP3 inserted²⁴. The FoxP3-Artichoke construct expresses a FoxP3-GFP fusion protein alongside a fluorescent mCherry reporter, allowing us to measure FoxP3 degradation through GFP fluorescence while using mCherry as an internal control for nonspecific degradation or cytotoxic effects (FIG. 8A). We first ensured the FoxP3-GFP fusion protein localized to the nucleus as expected (FIG. 8B). Next, we evaluated whether the fusion protein responded to biological perturbation. Indeed, overexpression of the bona fide FoxP3 endogenous E3 ligase, STUB1, dramatically inhibited FoxP3 protein expression without altering mCherry expression levels (FIG. 8C)²⁵. We then assessed chemical perturbation of the FoxP3-Artichoke HEK293T system using the high-content imaging technique that would be used for screening. As expected, chemical perturbation through proteasome inhibitor, MG132, increased percentage of GFP+ as well as mCherry intensity. Conversely, the pan-translational inhibitor cycloheximide (CHX) inhibited the FoxP3-GFP expression in the FoxP3-Artichoke HEK293T cells (FIG. 8D). However, we did not observe any decrease but a slight increase in mCherry levels when the FoxP3− Artichoke HEK293T cells were treated with CHX. This is possibly due to the relatively long half-life of mCherry protein and CHX-induced cellular toxicity causing autofluorescence (FIG. 8D). Nevertheless, these results indicate that CHX dramatically inhibited FoxP3-GFP fusion protein expression even before mCherry reduction. Collectively, our results confirmed that the FoxP3-Artichoke HEK293T cells are sensitive for screening of small molecules for FoxP3 protein degradation.

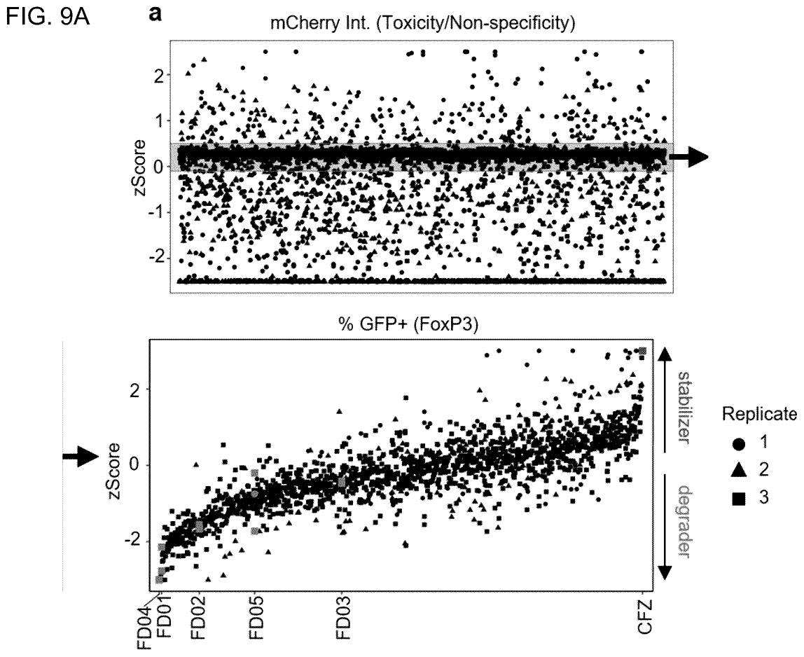

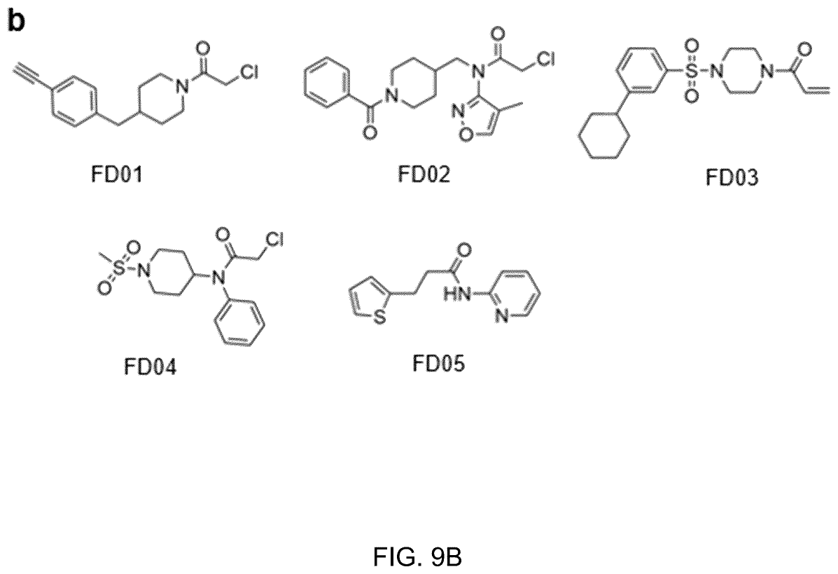

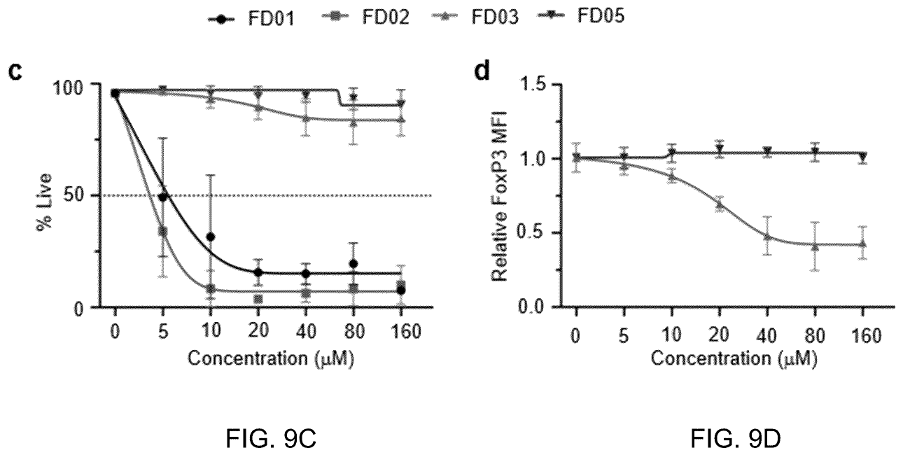

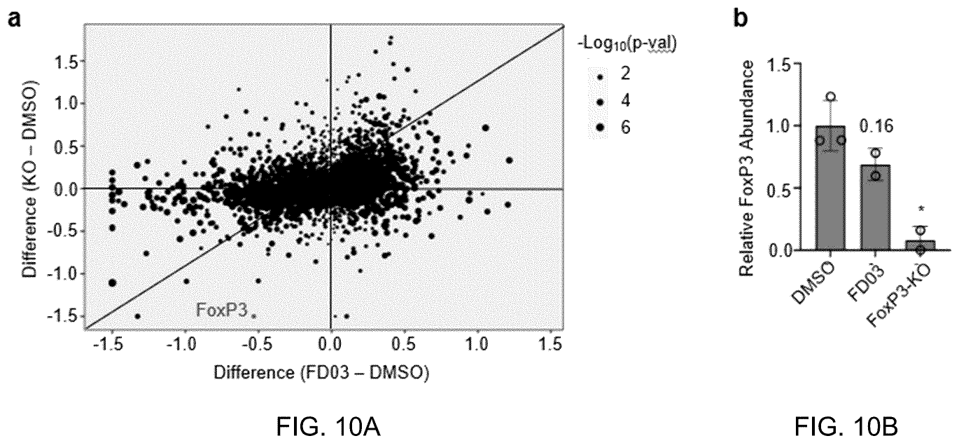

Using high-content imaging, we screened a targeted library containing 640 small electrophilic molecules for FoxP3 degradation (FIG. 1A). We first filtered out compounds that caused significant changes in mCherry fluorescence intensity, as this indicated non-specificity or cytotoxicity. We then ranked the remaining molecules based on changes in GFP fluorescence intensity (FIG. 9A). Next, 81 compounds were selected for further validation based on degradation ability and consistency between screens; following a targeted concentration response screen in FoxP3-Artichoke HEK293T cells, we selected twelve compounds for further analyses. We employed the human Treg-like cell line, MT-2, which endogenously expresses FoxP3, to further assess these twelve compounds by concentration response curves for cytotoxicity and FoxP3 reduction^{26,27} (FIG. 1A). These analyses identified five lead candidates as potential FoxP3 degraders (FIG. 1B, FIG. 9B). Since FD04, a fentanyl analog, could not be resynthesized, we proceeded with the remaining four lead compounds. After resynthesis, we found only FD03 and FD05 exhibited minimal toxicity, and FD03 was further confirmed with FoxP3-specific degradative activity in the FoxP3-Artichoke stable expression HEK293T cells (FIG. 1C, FIG. 9C). Importantly, resynthesized FD03 exhibited a potent degradative activity for the endogenous FoxP3 protein in human Treg MT-2 cells (FIG. 9D). In short, our high throughput screening identified FD03 as a potential FoxP3-specific small molecule degrader.

### FD03 is a Potent Degrader of FoxP3 in Mouse and Human Treg Cells.

To further assess FD03 activity, we performed Western blot analysis in MT-2 cells treated with varying concentrations of the compound. As expected, FD03 treatment showed decreased FoxP3 expression levels in the human, Treg-like MT-2 cells in a concentration dependent manner. We observed that FD03 treatment degraded FoxP3 with an EC₅₀ of roughly 20 mM; at 40 mM, FD03 treatment resulted in almost complete FoxP3 degradation (FIG. 1D). We then assessed FD03 in primary murine CD4+FoxP3+ Treg cells. Of note, our results indicate that, comparing to that in human, Treg-like MT-2 cell line, FD03 achieved an even better efficacy in reducing FoxP3 protein expression in mouse primary Treg cells, with an EC₅₀ of less than 10 mM. Moreover, treatment with FD03 at 40 mM did not exhibit any toxicities to mouse primary Treg cells, suggesting a large therapeutic window in Treg targeting to boost antitumor immunity (FIGS. 1E-1F). Consistently, FD03 also shows potent FoxP3 degradative capabilities in a dose-dependent manner in primary human Treg cells with minimal cell toxicity (FIGS. 1G-1H). These results indicate that FD03 is a FoxP3 degrader in both human and mouse Treg cells.

### FD03 Promotes Proteasomal Degradation of FoxP3.

Given we initially screened using a targeted degradation approach, we hypothesized that our lead molecule, FD03, inhibits FoxP3 protein expression through ubiquitination-mediated proteasomal degradation. To test this, we first examined whether proteasome inhibition could block FD03-induced FoxP3 reduction in MT-2 cells. Indeed, co-treatment with a pan-proteasome inhibitor, MG132, fully prevented FD03-mediated FoxP3 decrease, suggesting that FD03 acts through the proteasomal pathway for FoxP3 degradation (FIG. 2A). To support this conclusion, treatment of MT-2 cells with FD03 significantly facilitated FoxP3 protein degradation and shortened its half-life from more than 6 hours to less than 4 hours as determined by the cycloheximide (CHX) pulse chase experiment (FIG. 2B). Furthermore, FD03 increased poly-ubiquitination of FoxP3, consistent with its role in promoting ubiquitin-proteasome system-mediated degradation (FIG. 2C). Taken together, these results suggest that FD03 reduces FoxP3 protein levels through the ubiquitin-proteasome system.

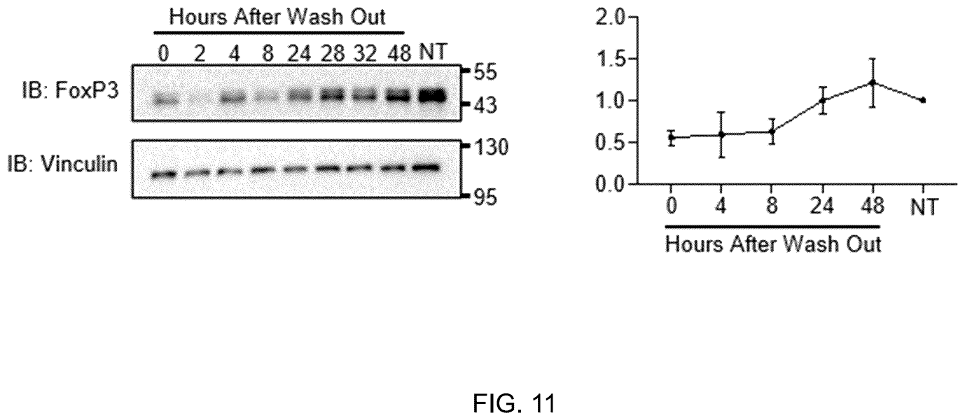

Given the crucial role of immunosuppressive Treg cells in maintaining immune homeostasis and preventing autoimmunity, we also sought to determine whether FD03-mediated FoxP3 degradation is a reversible process. Following FD03 washout, FoxP3 levels gradually recovered and returned to baseline within 24-48 hours, indicating that the effect of FD03 on FoxP3 degradation is reversible (FIG. 11).

### Degradation of FoxP3 by FD03 is Dependent on STUB1.

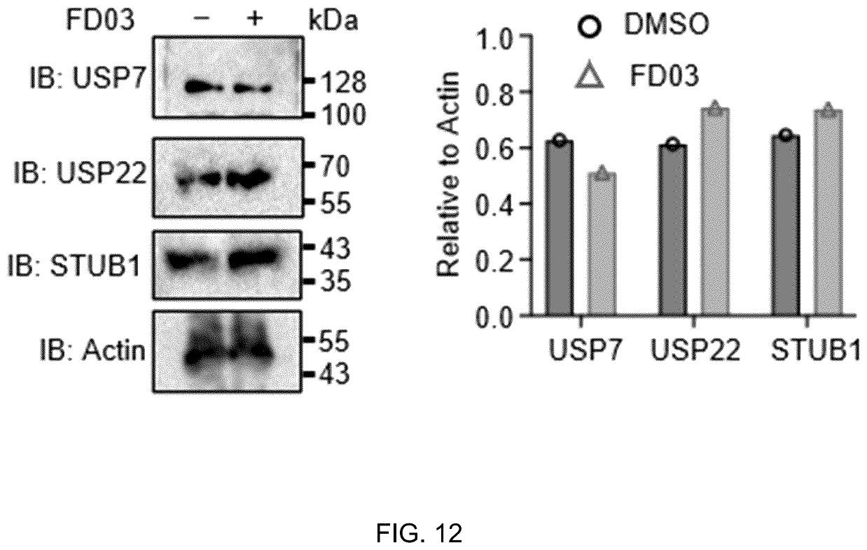

Next, we aimed to determine the molecular mechanism by which FD03 degrades FoxP3. As FD03 treatment enhances FoxP3 ubiquitination and proteasomal turnover, we first speculated whether FD03 manipulates FoxP3 crosstalk with the components in UPS known to regulate its stability, including the E3 ligase STUB1 (CHIP) and deubiquitinases: USP22, USP21, and USP7^{16,18,25,28,29}. The expressions of these deubiquitinases and E3 ligase were not affected or not detectable in MT-2 cells upon FD03 treatment (FIG. 12). Interestingly, a significant increase in STUB1 interaction with FoxP3 by FD03 treatment was detected in MT-2 cells, suggesting that FD03 degradation is mediated by, at least in part, promoting FoxP3 interaction with its bona fide E3 ubiquitin ligase STUB1 (FIG. 2D). To further probe this possibility, we generated STUB1 CRISPR-knock out (STUB1-KO) MT-2 cells. As expected, STUB1 ablation dramatically increased FoxP3 protein expression levels. Consistent with our interaction findings, we observed that genetic abatement of STUB1 largely abolished the FD03-mediated FoxP3 degradation (FIG. 2E). Altogether, this suggests that FD03 promotes FoxP3 degradation primarily by promoting its interaction with the E3 ligase STUB1.

Treatment with FD03 Partially Diminishes iTreg Immune Suppressive Function.

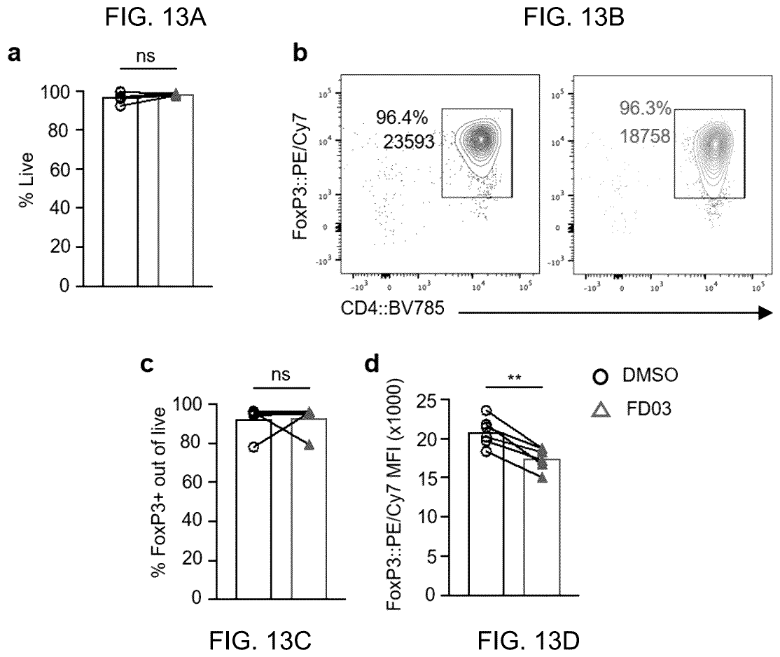

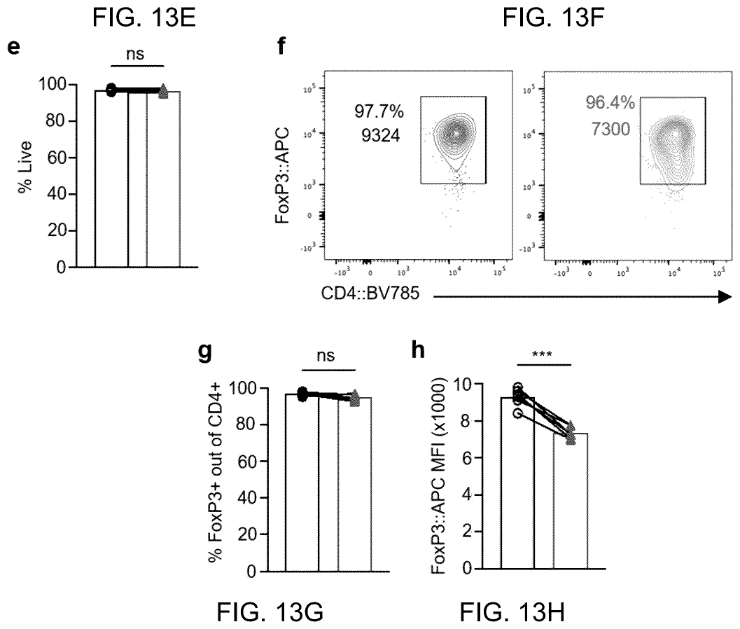

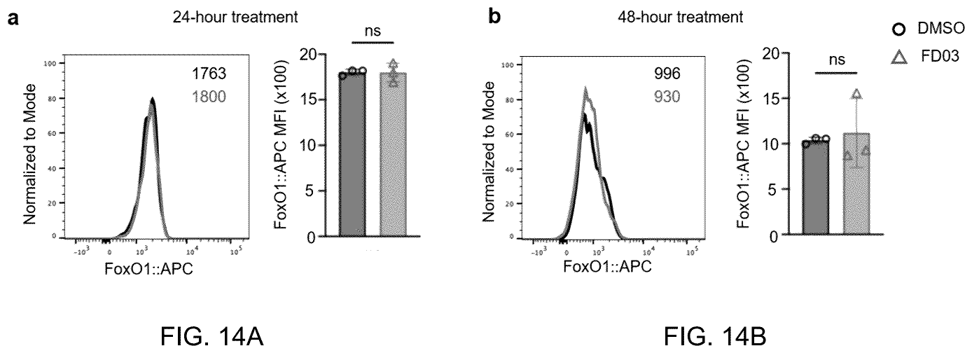

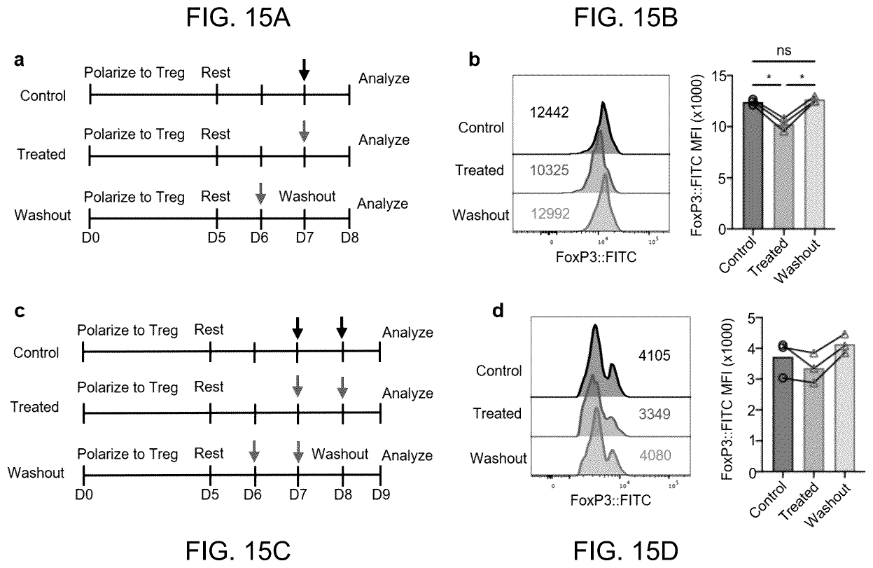

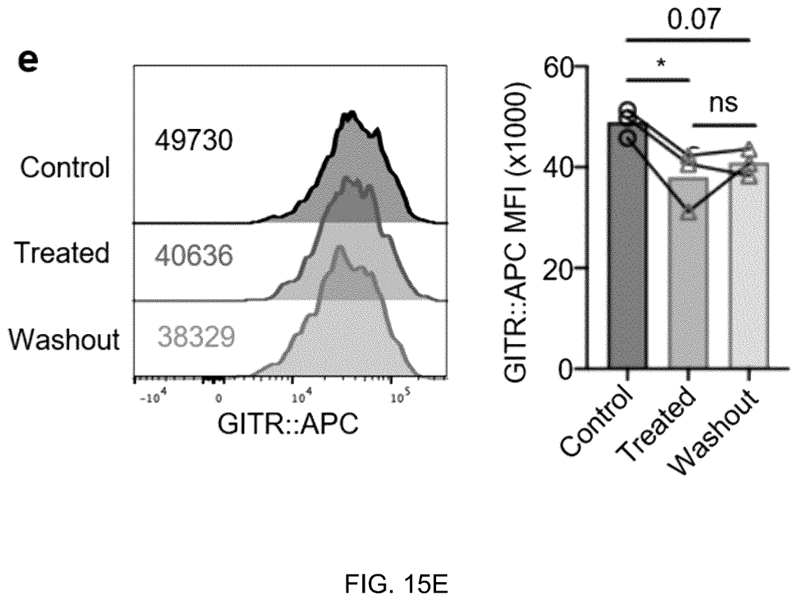

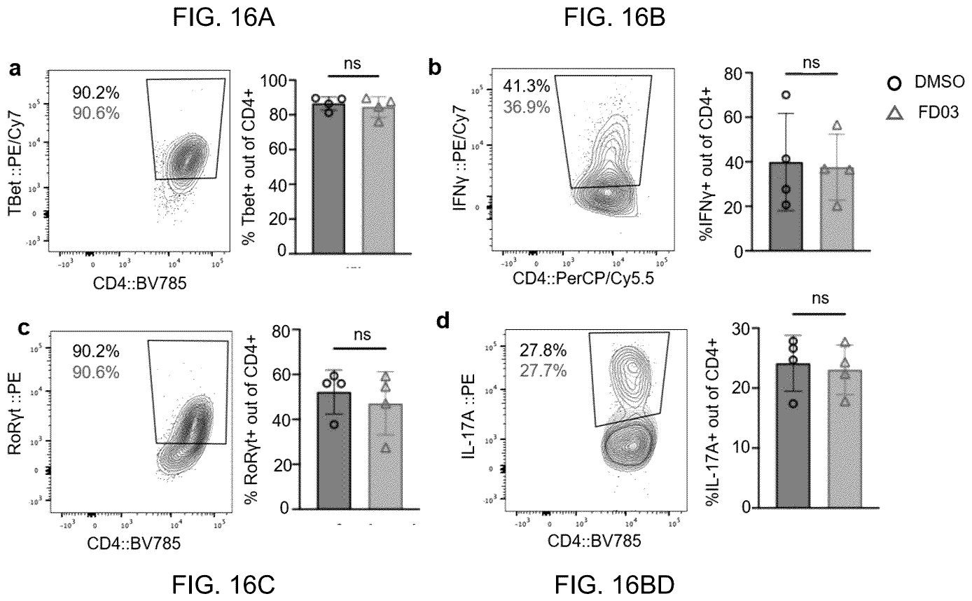

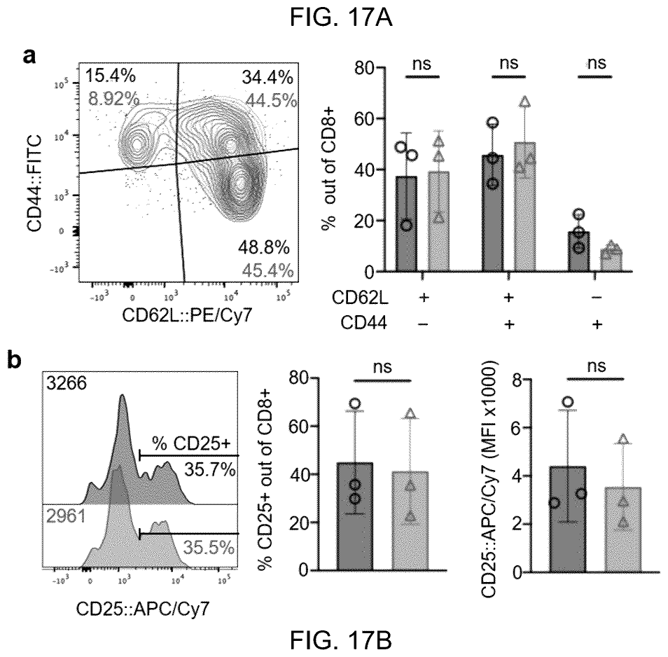

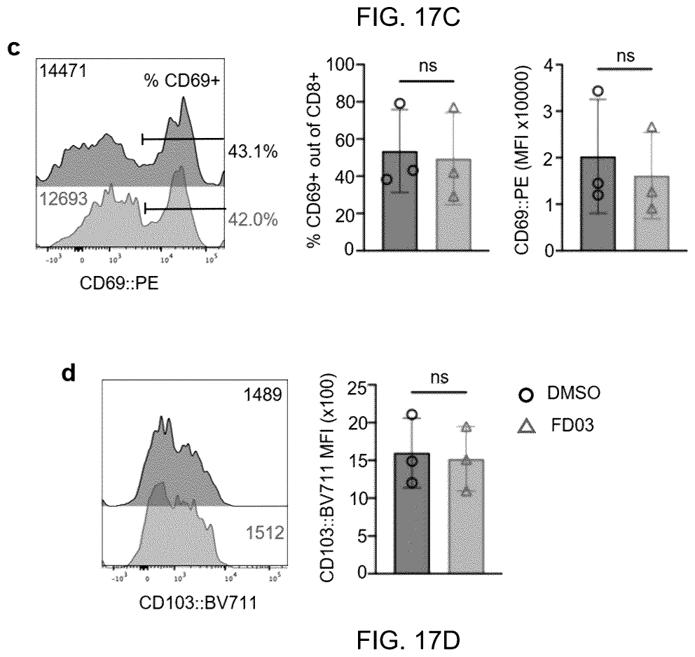

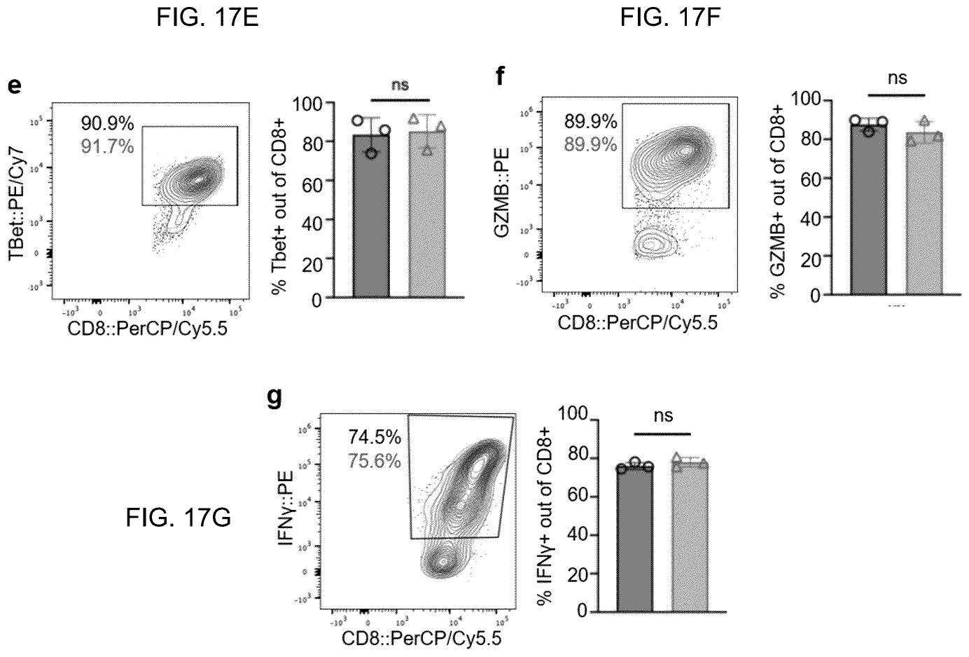

Given that FoxP3 is essential for Treg identity and dose-dependently maintains Treg immune suppressive functions, we assessed the impact of FD03 in Treg immune suppressive markers. Prior publications have suggested that a substantial portion intratumoral Treg cells are de novo polarized (PMID 23874336, 17277120). Therefore, we first analyzed in in vitro polarized Treg cells (iTreg cells). While we observed significantly decreased FoxP3 protein expression on a per cell basis in the FD03-treated iTreg cells compared to DMSO-treated cells, there were no differences in viability or FoxP3+ percentage between the DMSO- and FD03-treated iTreg cells (FIG. 3A, FIGS. 13A-13D). This suggests that these FD03-treated iTreg cells may lose iTreg identity and/or their suppressive function. Supporting this, FD03-treated iTreg cells exhibited reduced expression of suppressive surface marker GITR (CD357) along with increased CD25 expression (FIG. 3A). GITR, or glucocorticoid-induced TNFR-related receptor, has been shown to be influential in modulating the tumor immune response, largely through iTreg cells^{30,31} Therefore, this suggests that FD03 treatment potentially impairs the immunosuppressive functions of de novo polarized Treg cells in a tumor setting.

To further evaluate whether FD03 treatment impairs iTreg suppressive capacity, we performed an in vitro suppressive assay by co-culturing DMSO- and FD03-pretreated iTreg cells with CD8+ T cells. Critically and consistent with our earlier findings, FD03 pretreatment did not reduce viability or FoxP3+ iTreg frequency but resulted in a significant reduction in FoxP3 MFI in iTreg cells (identified by CD4+FoxP3+) (FIGS. 13E-13H). To track CD8+ T cell proliferation, the T effector (Teff) cells were stained with Cell Trace Violet (CTV). As the cells divide, the CTV intensity is diluted at each division cycle allowing us to observe how the proliferative capacity through number of or size of CTV peaks at different intensities. Indeed, we observed increased number of peaks as well as a much larger peak at a lower MFI in the T_{eff} cells co-cultured with FD03-pretreated Treg cells, indicating there was increased proliferation of these T_{eff} cells due to the impaired Treg suppressive functions (FIG. 3B). We performed division index analyses of CTV-treated T_{eff} cells; our analyses revealed that there was significantly increased division of CD8+ T cells cocultured with FD03-pretreated Treg cells, indicating FD03 treatment decreased Treg-mediated suppression (FIG. 3B). Together, these findings suggest that FD03 destabilizes FoxP3 expression in Treg cells and impairs their ability to suppress CD8+ T cell proliferation, highlighting the potential of FD03 to enhance CD8+ T cell antitumor immunity by targeting FoxP3+ Treg functions.

Lastly, given murine iTreg cells may not faithfully recapitulate human iTreg cells, we assessed the suppressive markers in human iTreg cells treated with multiple doses of FD03. As expected, we observed a significant decrease of FoxP3 protein expression in FD03-treated compared to DMSO-treated iTreg cells (FIG. 3C). Unlike murine iTreg cells, FD03 treatment in human iTreg cells notably decreases the proportion of FoxP3+ cells upon treatment in concentration-dependent manner. This is likely due to relatively lower levels of FoxP3 expression in the human iTreg cells following in vitro polarization compared to murine iTreg cells. To fairly assess the effects of FD03 given that FD03 treatment, we performed subsequent analyses from the total CD4+ T cell population. We observed an increase in GITR expression and decrease in CD25 expression upon FD03 treatment (FIGS. 3D-3E). Unlike murine iTreg cells, which had no changes in PD-L1 or CTLA-4 expression, we found that FD03 treatment significantly increased PD-L1 expression and decreased CTLA-4 expression as well as PD-1 expression (FIGS. 3G-3H).

FD03 Inhibits nTreg Cell Immunosuppressive Function in an IFN-γ Dependent Manner.

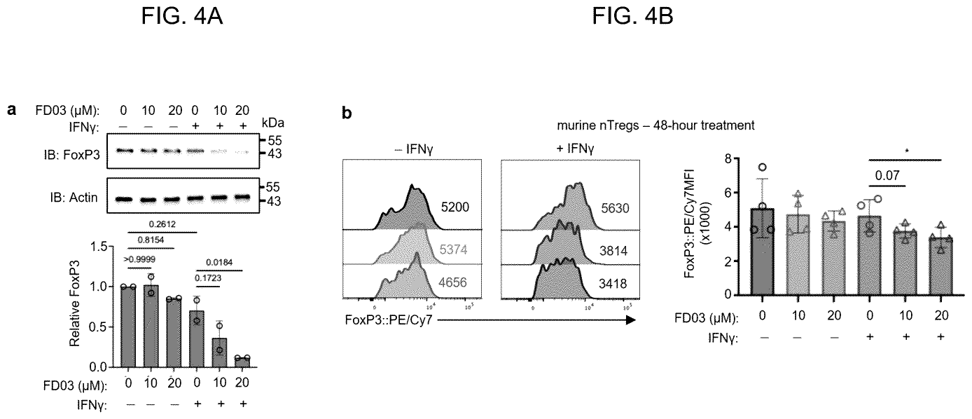

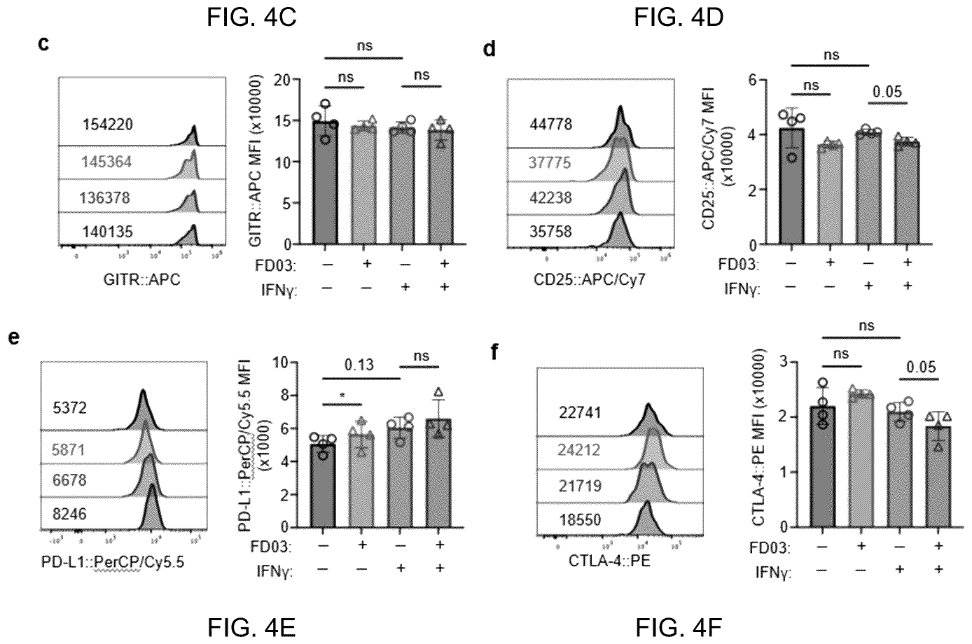

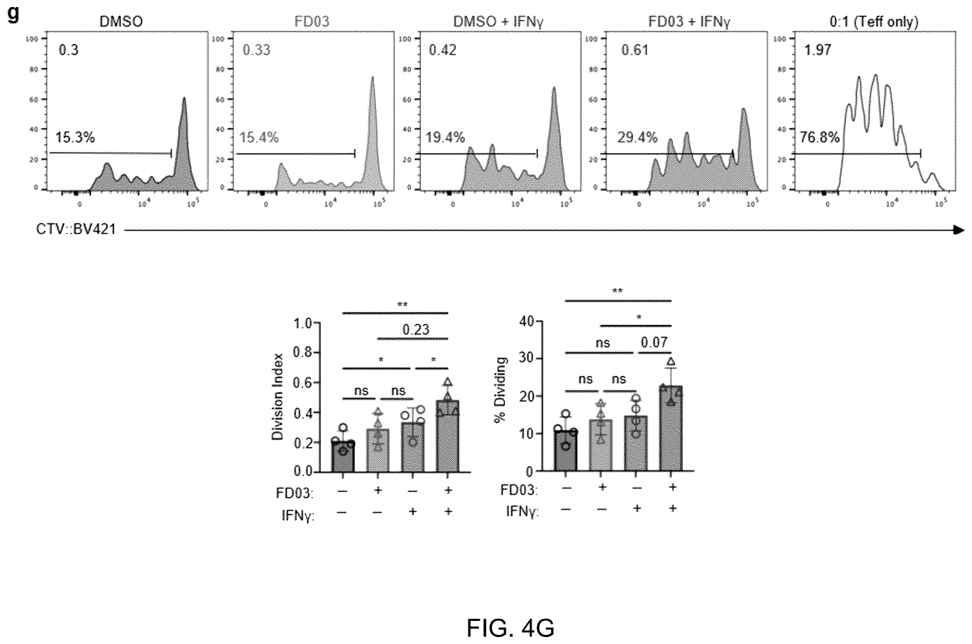

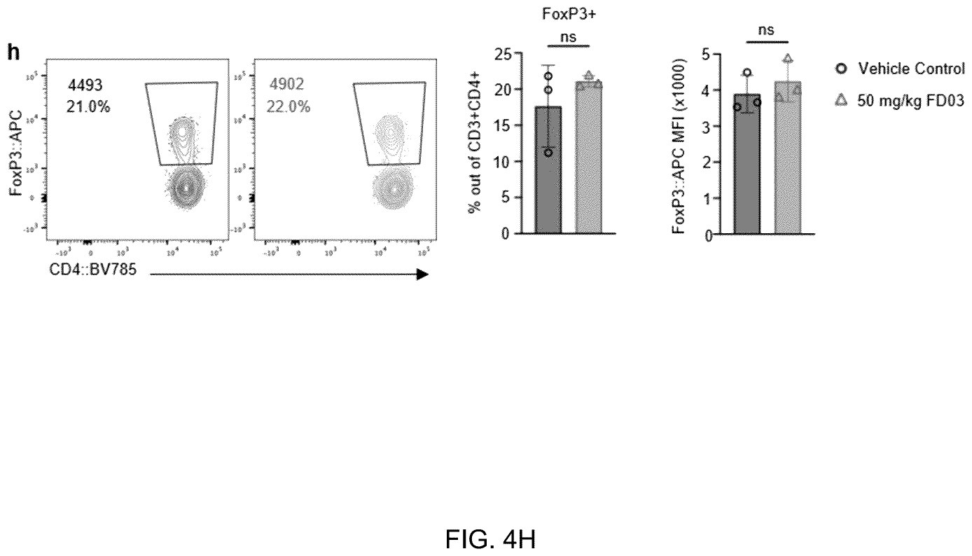

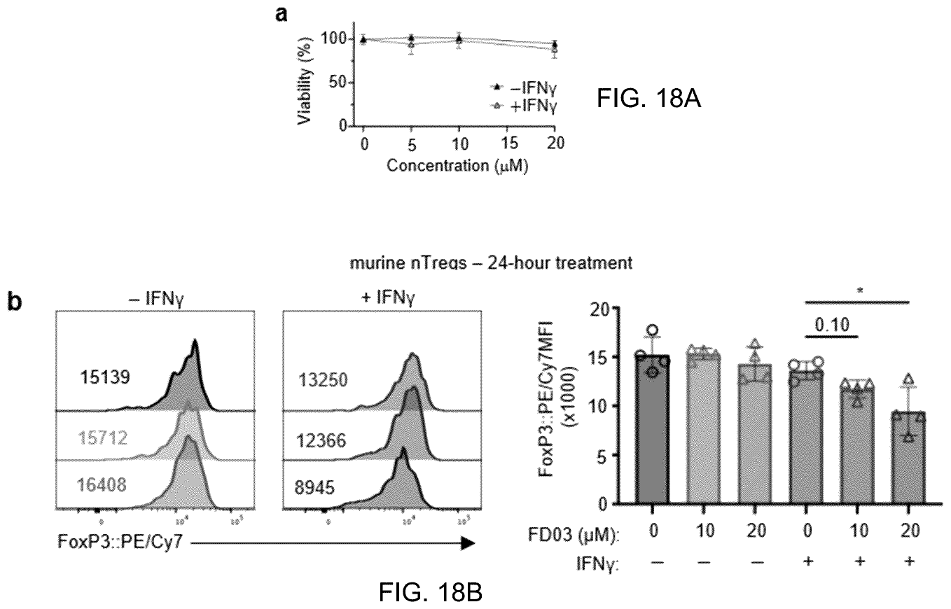

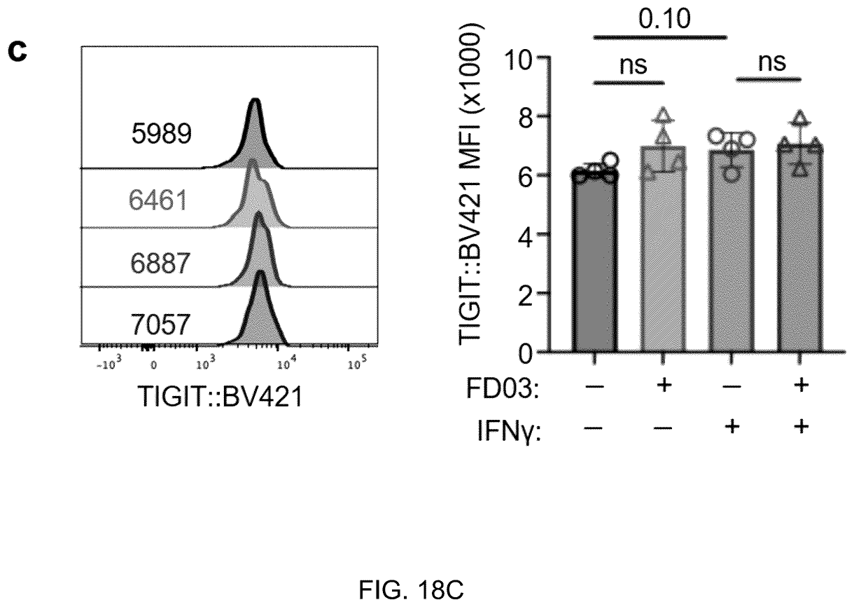

Naturally occurring Treg (nTreg) cells are typically thought to have increased stability compared to iTreg which are thought to have increased plasticity. Due to increased stability of Treg identity, we sought to further characterize effects of FD03 treatment on nTreg cells. Surprisingly, at concentrations where we previously observed 50% reduction in FoxP3 protein expression in iTreg cells, we did not observe any significant degradation at 10 or 20 μM FD03 treatment in murine nTreg cells (FIGS. 4A-4B, FIG. 18B). To mimic the tumor microenvironment which often has increased levels inflammatory cytokines such as interferon-gamma (IFN-γ) and IL-6, we then treated the nTreg cells in the presence of IFN-γ. Excitingly, FD03 treatment decreased FoxP3 protein expression in a dose-dependent manner only in the presence of interferon-gamma (FIGS. 4A-4B, FIG. 18B).

We then assessed the effects of FD03 treatment on n Treg cell immunosuppressive function through known markers and suppressive capabilities with and without presence of IFN-γ. Unlike murine iTreg cells, we did not observe any differences in GITR expression regardless of IFN-γ (FIG. 4C). However, consistent with our FoxP3 degradation results, we only observed significant differences in nTreg cells treated with both FD03 and IFN-γ compared to both DMSO-only control and DMSO/interferon-gamma treatment. FD03 and IFN-γ co-treatment resulted in decreased CD25 expression as well as increased PD-L1 expression (FIGS. 4D-4E). Additionally, co-treated nTreg cells also had significantly decreased CTLA-4 expression (FIG. 4F). Treg

To evaluate nTreg cell suppressive capabilities, we pretreated murine n Treg cells in the same conditions and then co-cocultured with CD8+ T cells stained with CTV to track the CD8+ T_{eff} cell proliferation. nTreg cells pretreated with FD03 alone did not have decreased suppressive capability compared to DMSO-control treated cells. We observe a modest increase in division index of T_{eff} cells cocultured with IFN-γ-treated nTreg cells compared to DMSO-control which was expected as interferon-gamma is a known destabilizer of Treg function. Most notably, we see a significant increase in T_{eff} proliferation when the CD8+ T cells are co-cultured with FD03 and IFN-γ co-treated nTreg cells (FIG. 4G). This further confirms that in nTreg cells, FD03 requires the presence of IFN-γ to diminish immunosuppressive function.

Given our results and prior findings from Chen, et al showing that STUB1 expression could be induced by inflammatory molecules such as IL-1β and LPS in nTreg cells, we postulated that IFN-γ treatment promotes STUB1 expression in nTreg cells to facilitate FoxP3 degradation through FD03. Indeed, we found increased STUB 1 protein levels in nTreg cells cultured with IFN-γ compared to without (FIG. 4H).

### FD03 Treatment Does Not Impair Treg Cells at Steady State In Vivo

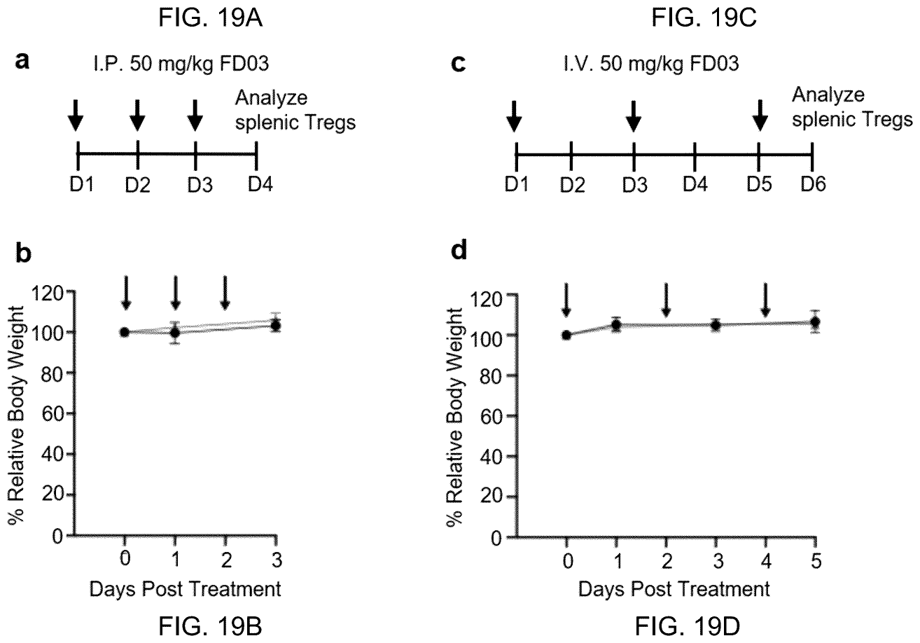

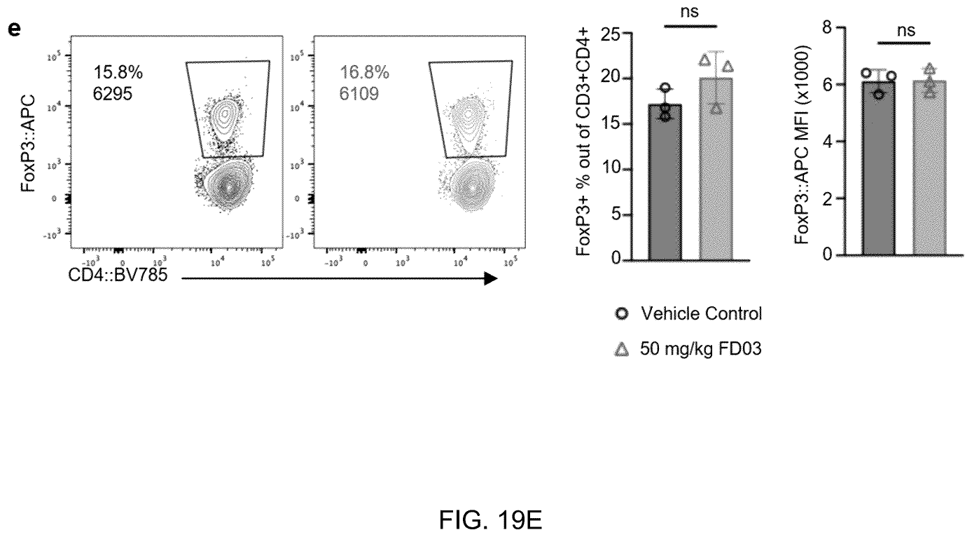

We initially attempted systemic administration of FD03 via either intraperitoneal or intravenous injection into mice; however, this approach did not result in any significant changes in the percentage or FoxP3 protein levels of splenic Treg cells (FIG. 4H, FIG. 19E). Importantly, we observed no acute toxicities dose of FD03 suggesting there is a large therapeutic window for treatment (FIGS. 19B-19D).

### FD03 Treatment Reduces Tumor Growth and Burden in Mice at a Clinically Relevant Setting.

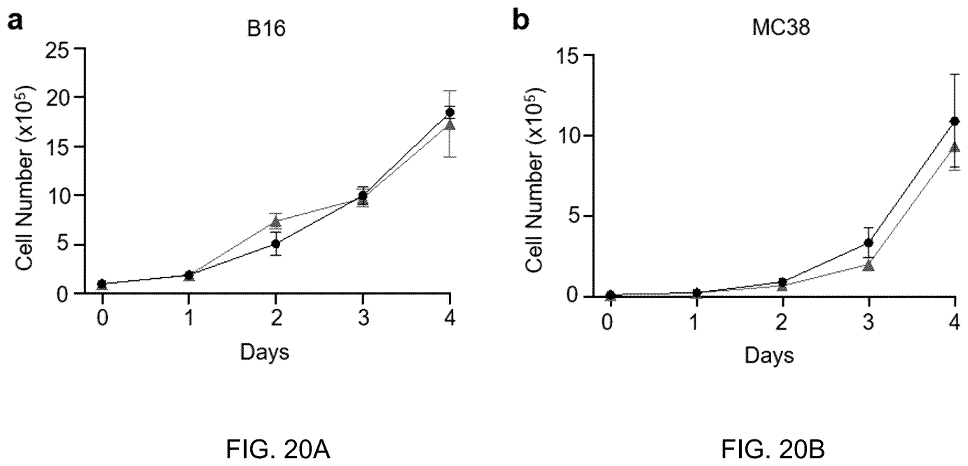

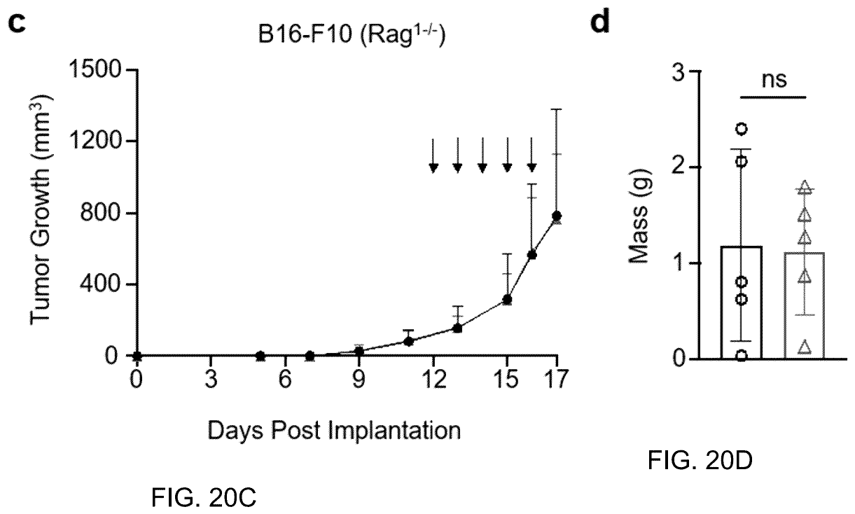

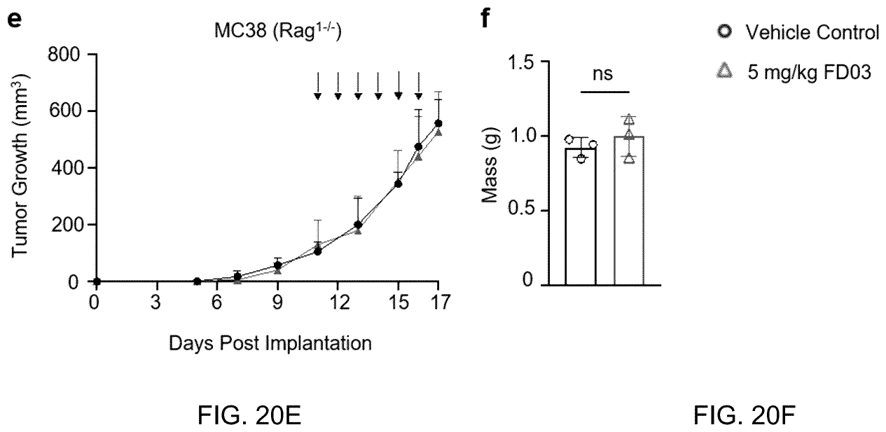

As an immune checkpoint, Treg infiltration and accumulation within solid tumors leads to tumor immune evasion and is responsible for the current antitumor immunotherapeutic resistance^{4,5,8,32,33}. Therefore, Treg cells have been proposed as a therapeutic target to boost antitumor immunity. We then sought to assess the therapeutic efficacy of FD03 in a preclinically relevant syngeneic tumor model. We initially attempted systemic administration of FD03 via either intraperitoneal or intravenous injection into mice; however, this approach did not result in any significant changes in the percentage or FoxP3 protein levels of splenic Treg cells (FIG. 19E). Importantly, we observed no acute toxicities associated with high dose of FD03 suggesting there is a large therapeutic window for treatment (FIGS. 19B-19D). Therefore, we then performed preclinical studies with local administration of FD03 into pre-established subcutaneous tumor models. We found that there was significantly decreased tumor growth and tumor burden upon treatment of FD03 in multiple tumor types, B16-F10 melanoma, EG7 lymphoma, and MC38 colon carcinoma (FIGS. 5A-5F). Furthermore, we found no difference in tumor cell growth upon in vitro treatment with FD03 versus DMSO, suggesting that FD03-mediated antitumor activity is tumor cell-extrinsic and likely driven by Treg targeting (FIGS. 20A-20B).

### FD03 Treatment Promotes T Cell Mediated Anti-Tumor Immunity Through Modulation of Treg Cells.

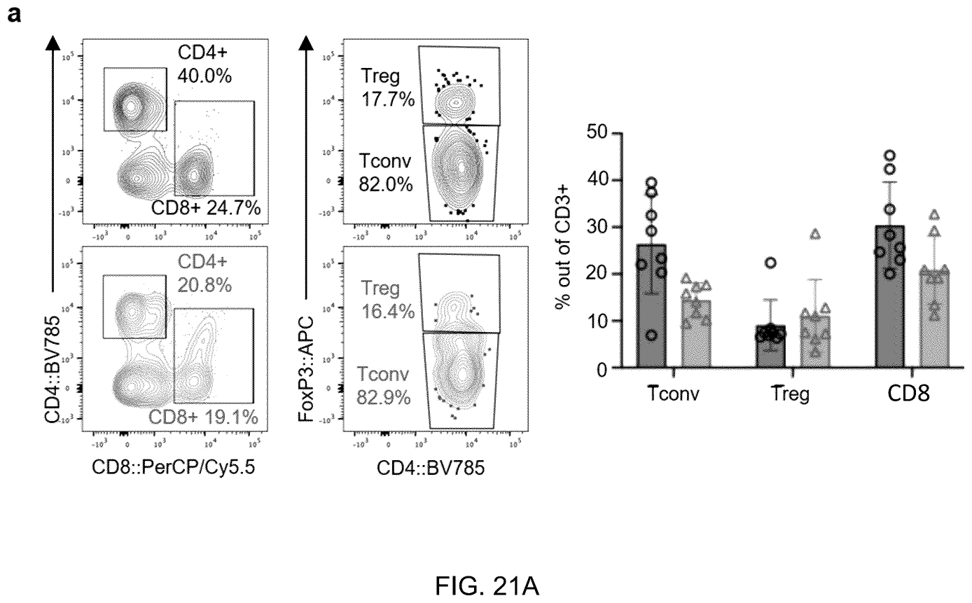

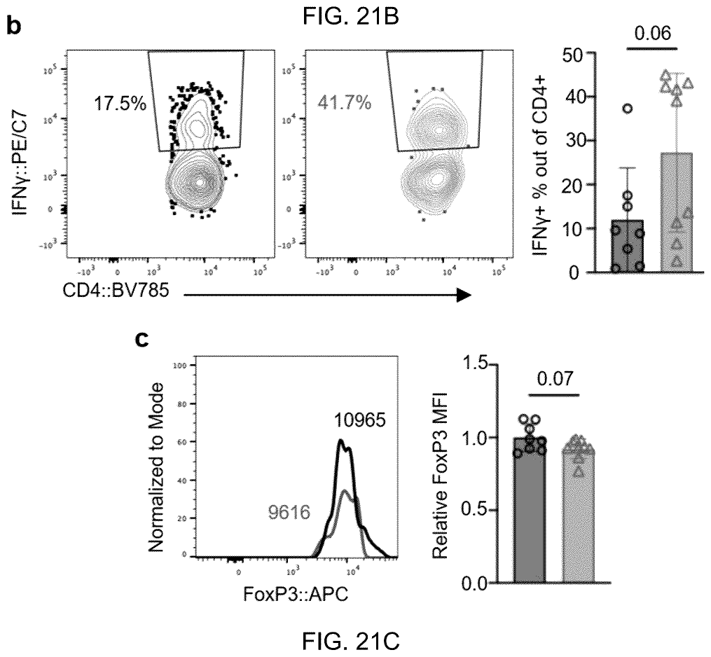

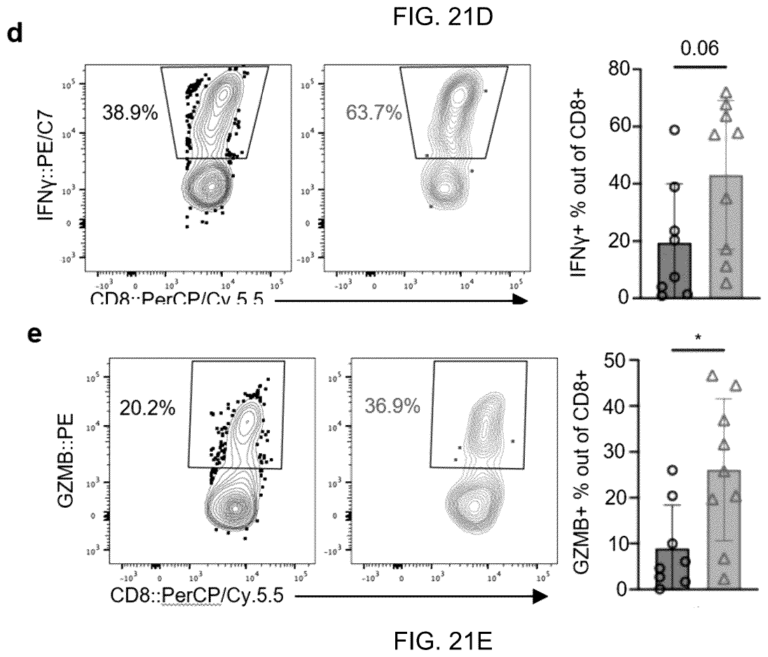

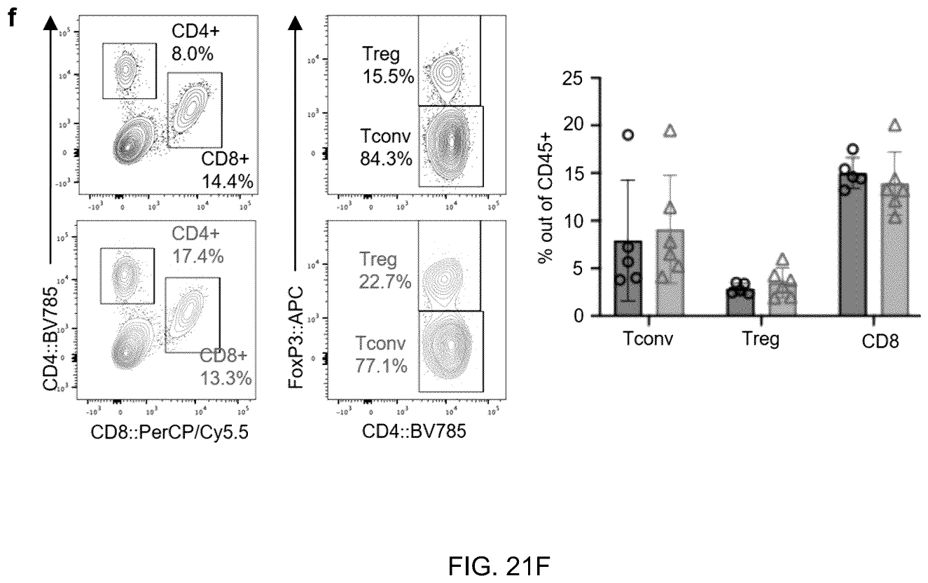

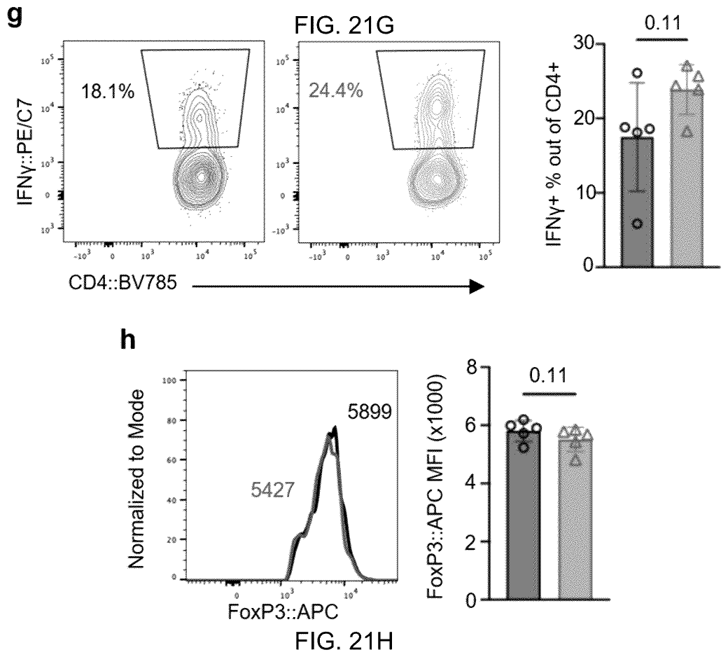

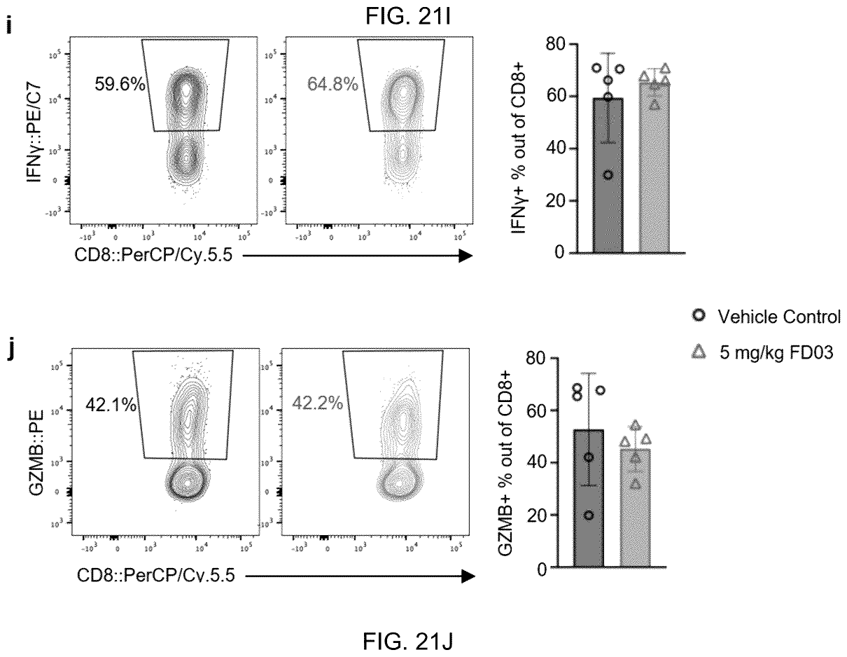

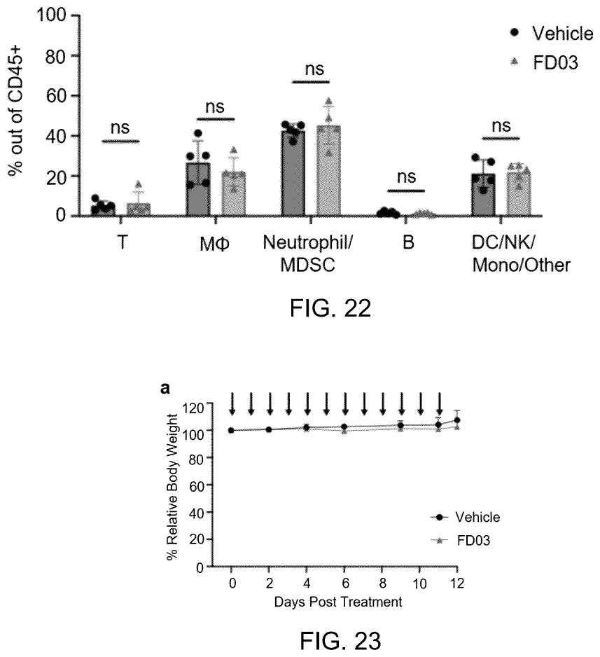

Next, we analyzed the tumor-infiltrating lymphocytes to assess changes in the immune landscape associated with Treg targeting by FD03. We observed there was a trending increase in overall lymphocyte (gated on CD45+) infiltration in the FD03-treated tumors (FIG. 5G). We also observed a trending decrease in Treg cells (gated as CD4+FoxP3+) in the tumor and increased T conventional cells (Tconv, gated as CD4+FoxP3−) as well as cytotoxic CD8+ T cells (gated as CD8+) (FIG. 5H). More fascinatingly, we observed a significant increased frequency of IFNg-producing CD4+ cells in FD03-treated tumors compared to DMSO-treated tumors (FIG. 5I). Most promisingly, FD03 treatment resulted in a significant increase of inflammatory molecule, IFN-γ, from CD8+ T cells, with a trend towards increased production of the cytotoxic molecule, granzyme B (GZMB) (FIGS. 5J-5K). This suggests that FD03 treatment may be skewing the intratumoral CD4+ T cell population towards a less suppressive, more inflammatory and antitumoral phenotype and promotes CD8+ T cell anti-tumoral function. We also assessed T cell function in other tumor types, and we found a similar increase in tumoral T cell function in both IFNg and/or granzyme-B production in CD4+ and CD8+ T cells (FIG. 21). Altogether, this supports that FD03 treatment through targeting Tregs through FoxP3 has therapeutic efficacy across multiple tumor types.

Given that FD03 targets FoxP3 and Treg cells, we sought to further characterize any changes in intratumoral Treg cells by assessing Treg-specific markers. We first assessed FoxP3 protein levels by intracellular staining, and we observed a notable decrease in FoxP3 levels in the intratumoral Treg cells of mice treated with FD03 (FIG. 5L). Aligned with our in vitro findings, we also detected significantly decreased GITR expression in the FD03-treated mice (FIG. 5M). Another known suppressive ligand of Treg cells, TIGIT, also showed a trend toward both lower frequency and reduced expression levels as measured by MFI (FIG. 5N), while Helios expression remained unchanged (FIG. 5O). Importantly, both GITR and TIGIT have come to light under recent years as potential new immunotherapeutic targets as these ligands, particularly their expression on intratumoral Treg cells, due to their roles in potentiating intratumoral Treg function and dampening the anti-tumor response^{30,31,34-38}. Taken together with our findings, this suggests that FD03 treatment can promote the anti-cancer immune response through amelioration of multiple axes of Treg suppression by targeting FoxP3 protein degradation.

Finally, to confirm that the decrease in tumor growth in FD03 treated mice is T cell mediated, we repeated our initial tumor growth experiments in Rag1^{-/-} mice which lack T cells. Indeed, we found that there was no difference in tumor growth or burden following FD03-treatment compared to vehicle control in Rag1^{-/-} mice bearing either B16 melanoma or MC38 colon carcinoma tumors (FIGS. 20C-20F), further confirming that FD03-mediated antitumor activity is tumor cell-extrinsic and driven by Treg targeting.

FD03 Synergizes with Immune Checkpoint Blockade Therapy

Given that ICB therapies often result in incomplete remission or tumor progression due to Tregs, we also evaluated whether FD03 can enhance the effects of ICB. As expected, anti-PD-1 single treatment did not significantly impair the growth of ICB-resistant LLC-1 lung carcinoma. FD03 single treatment modestly decreased tumor growth in comparison to vehicle treated control. Most promisingly, FD03 and anti-PD-1 combination treatment significantly ameliorated tumor growth compared to vehicle control, FD03 single treatment, and anti-PD-1 treatment (FIGS. 5P-5Q). Altogether, this data shows that targeting tumoral Tregs through FoxP3 can enhance existing ICB therapies.

### Systemic FD03 Treatment is Specific to Tumoral Tregs

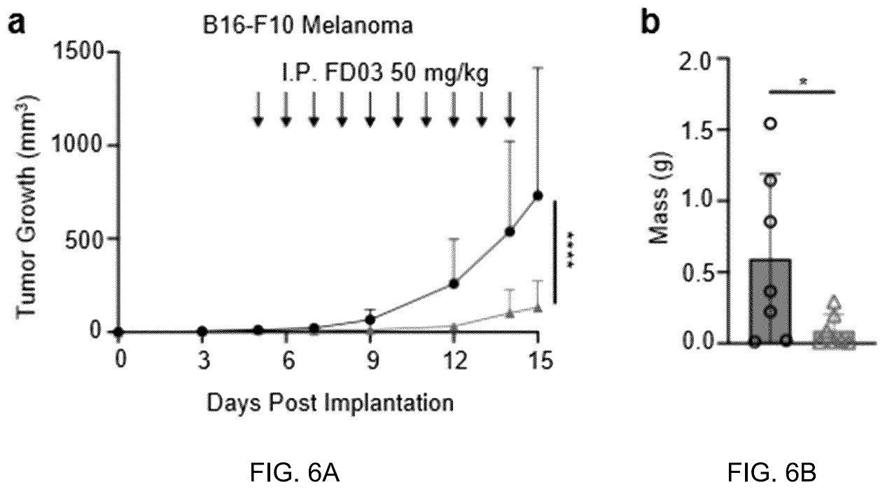

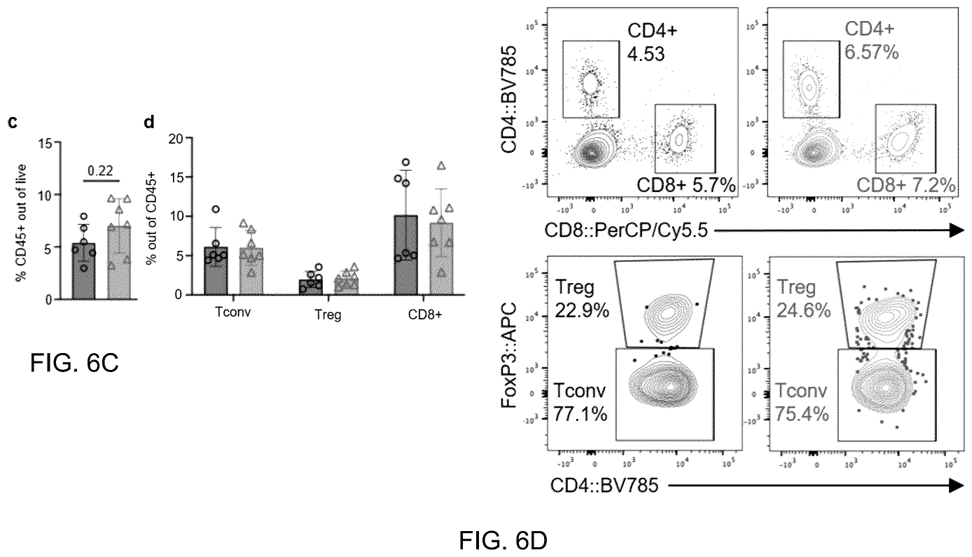

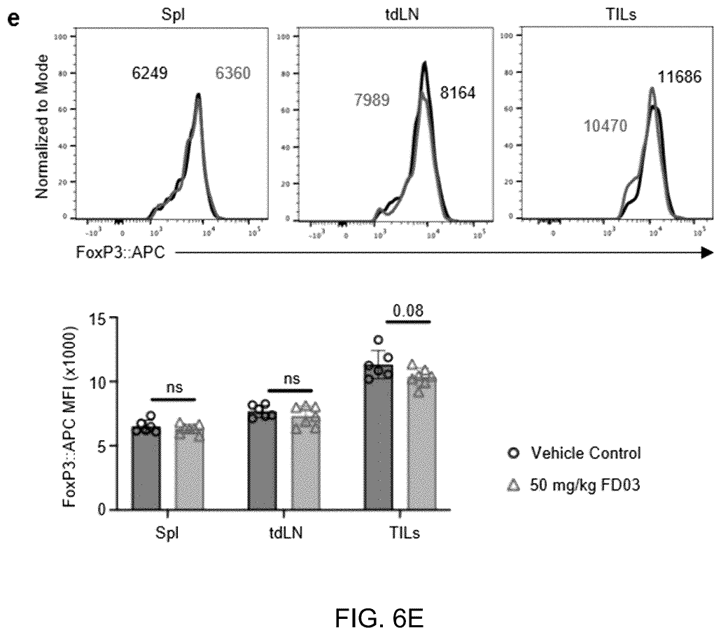

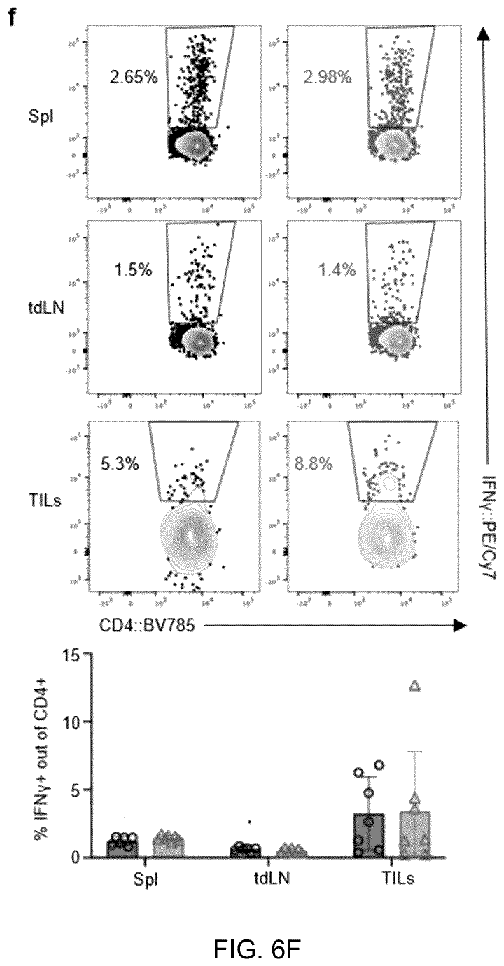

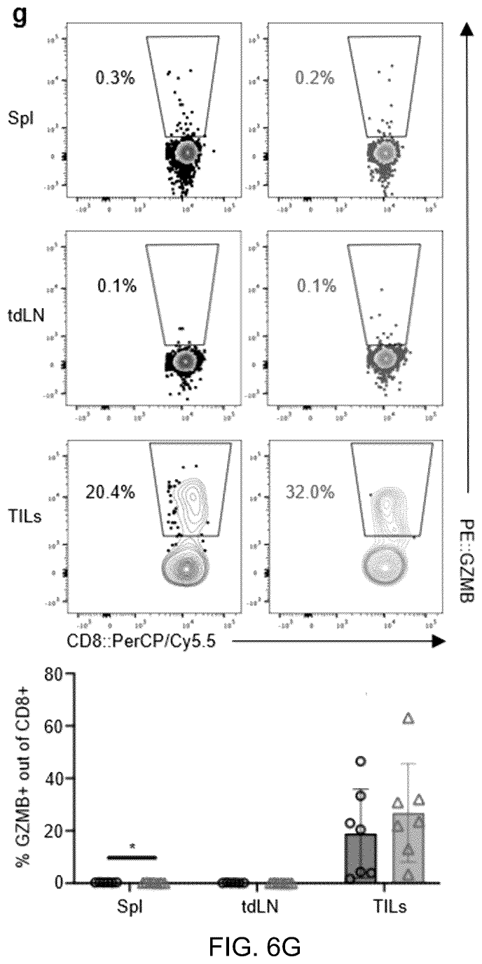

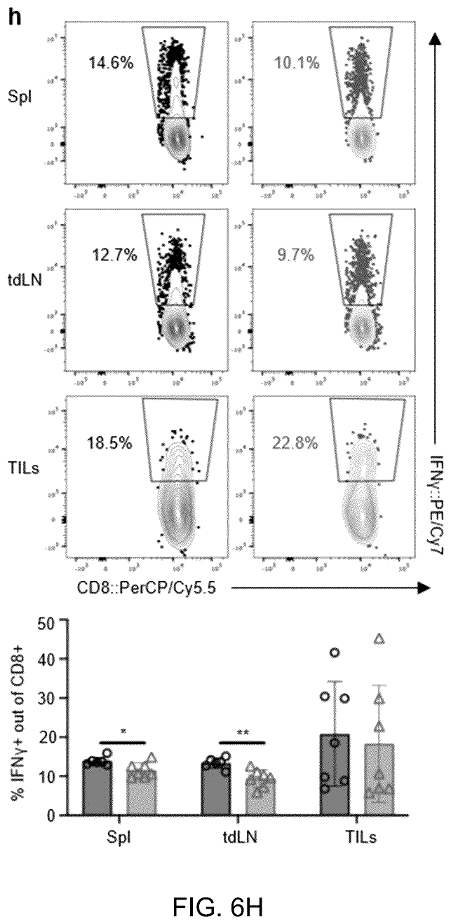

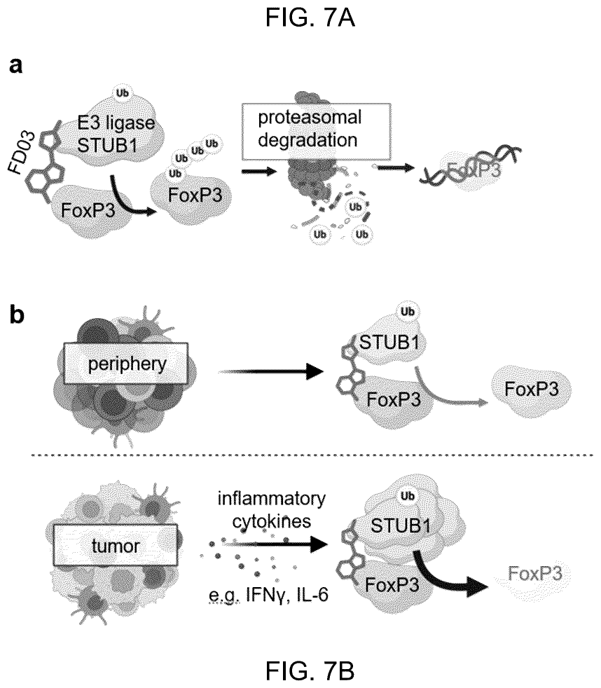

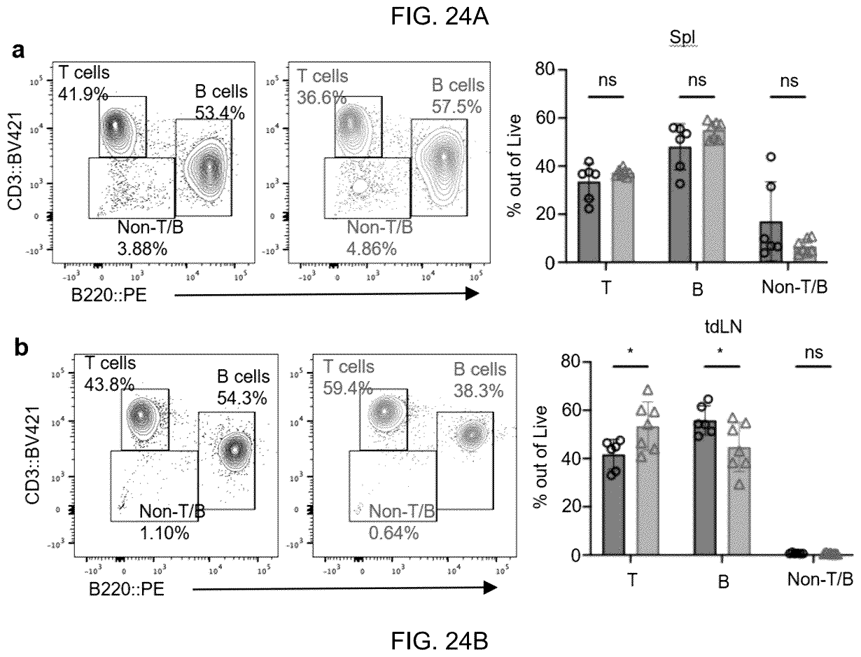

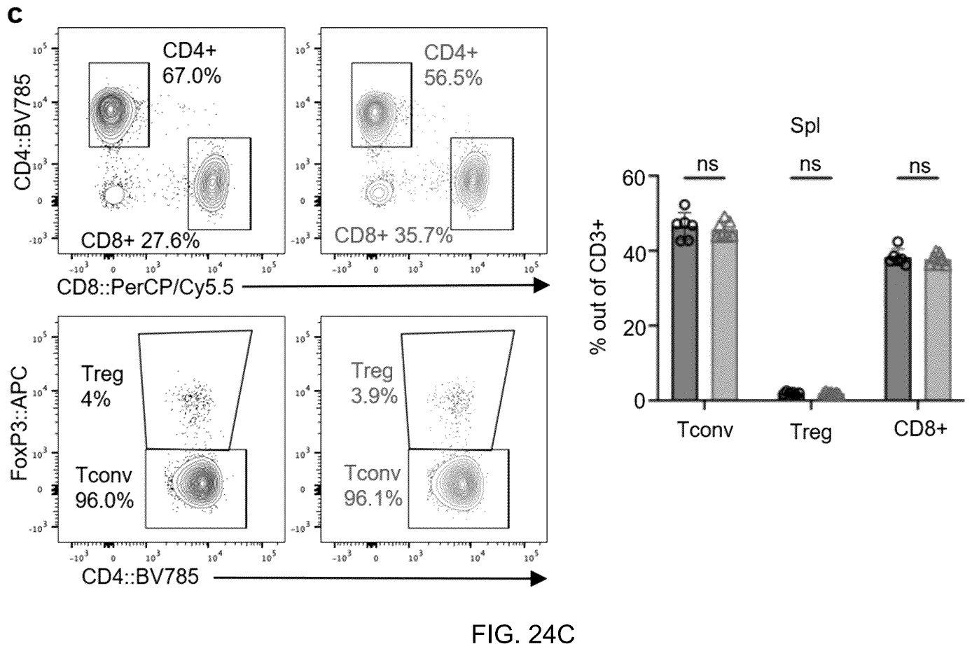

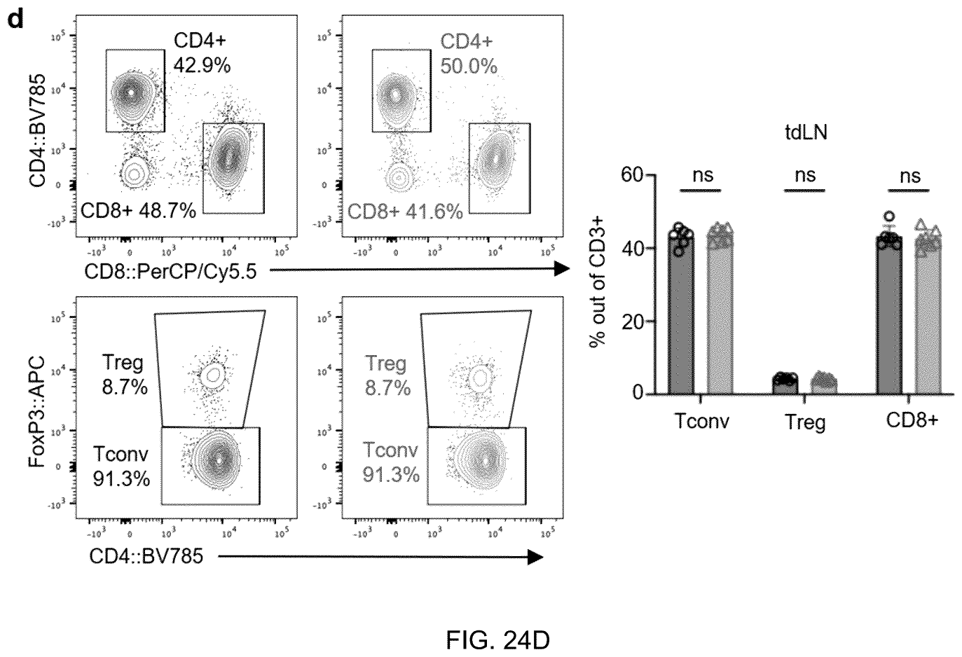

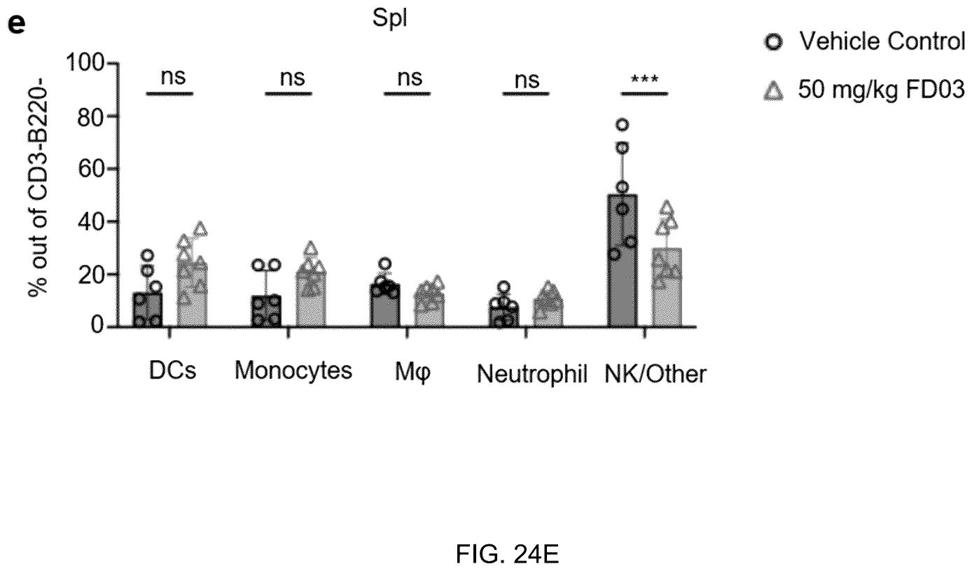

Finally, we asked whether systematic treatment of FD03 could specifically affect tumoral Tregs given that the inflammatory nature tumoral microenvironments compared to peripheral lymphoid organs. Surprisingly, we found that treatment via I.P. administration of FD03 in tumor-bearing mice was sufficient to significantly slow tumor growth and decrease tumor burden without any acute toxicity (FIGS. 6A-6B, FIG. 23). In the tumor, we see an increase of CD45+ tumor infiltrating lymphocytes suggesting systematic FD03 treatment can improve anti-tumor immunity compared to vehicle control treatment (FIG. 6C). We also evaluated other splenic and tumor-draining lymph node (tdLN) populations (FIG. 24). The most notable change is an increase of overall T cell proportion in tdLNs (FIG. 24). Similarly to peritumoral treatment, we do not see notable differences in the T cell composition (FIG. 6D). Most excitingly, when we further evaluated the Treg cells, we only see a reduction in tumoral Tregs but not splenic or (tdLN) Tregs (FIG. 6E). We also note that there is a trending increase in T cell function through IFNg or GZMB production of tumor-infiltrating CD4+ and CD8+ T cells which mirrors our previous findings from peritumoral FD03 treatment (FIGS. 6F-6H). In short, these findings in conjunction with our prior results suggest that FD03 treatment has a degree of specificity in vivo due to the mechanism by which FD03 degrades FoxP3.

While we found that 50 mg/kg of FD03 was efficacious, both the possibilities of sufficiency at decreased dose or increased efficacy at a higher dose remain to be explored.

### Discussion

Unfortunately, Phase I clinical trials of AZD8701 monotherapy did not show any objective responses in patients and many patients treated with combination of the ICB, durvalumab, and AZD8701 experienced treatment-emergent severe adverse events [PMID 39937271].

Historically, while Treg cells have been scrutinized for their strong effect in the anti-tumor immune response and pivotal role in immune checkpoint blockade patient outcomes, there have been little to no methods for precision targeting of Treg cells^{4,5,7,8,39,40} Herein, we illustrated the identification and characterization of a small molecule degrader FD03 that targets both human and murine FoxP3 for proteasomal degradation, thereby destabilizing Treg function. Moreover, we demonstrate FD03 has in vivo efficacy in suppressing tumor growth in a clinically relevant setting by diminishing intratumoral Treg immune suppressive functions through promoting FoxP3 proteasomal degradation. Notably, FD03-mediated FoxP3 degradation is reversible, offering the potential to mitigate immune-related adverse effects by discontinuing treatment if necessary. Looking towards the future, our findings not only provide a framework for the rational design of more potent FoxP3-targeting degraders or PROTACs but also offer novel insights into the regulation of FoxP3 and Treg biology.

Given the difficulties of pharmacologically targeting FoxP3, there have been limited studies on how targeting FoxP3 at the protein level can affects Treg identity. FoxP3 has been pharmacologically targeted through a peptide inhibitor, P60, and anti-sense oligonucleotide, AZD8701^{41,42}. Wang, et al show a P60-pomalimod PROTAC-like drug can degrade FoxP3 at the protein level but did not elaborate on the biological effects of perturbing FoxP3 at the protein level in Treg cells⁴³. Our lab has also shown decrease in FoxP3 levels through inhibition of its endogenous deubiquitinase, USP22; however, USP22 can also modulate FoxP3 transcriptional levels and USP22 is expressed ubiquitously throughout the body^{16,18,44-46}. To our knowledge, FD03 represents the first small-molecule degrader capable of directly targeting FoxP3 protein for proteasomal degradation in both human and murine primary Treg cells. This allowed us to characterize, for the first time, the cellular and functional consequences of partial FoxP3 reduction at the protein level. However, systemic administration of FD03 failed to modulate Treg cells under baseline conditions. There are several possibilities such as rapid metabolism and poor solubility stemming from the hydrophobic nature of FD03. As such, further optimization of FD03 is needed to enhance its pharmacokinetic properties and therapeutic potential, both as a candidate for immunotherapy and as a research tool to study FoxP3-dependent Treg biology.

Nevertheless, it is well documented that the high intratumoral Treg infiltration is associated with poor clinical outcomes in many types, if not all human tumors^{18,47}. Recent clinical studies revealed that the elevated accumulation is responsible for the antitumor immune therapies including immune checkpoint blockade such as by anti-PD-1/PD-L1 and anti-CTLA-4^{8,18,33,39,48} Experimental targeting FoxP3 or Treg instability such as by genetic approaches results in significantly improved tumor outcomes^{4,16,18,49}. Williams and Rudensky showed that while mice with acute complete genetic ablation of FoxP3 eventually develop autoimmunity due to loss of Treg function, the autoimmune phenotype is not immediate, and mice are able to survive for prolonged periods of time¹⁷. Moreover, we have recently shown that modest reduction, 20-30% decrease, in FoxP3 expression is sufficient to boost antitumor immunity without causing immune-associated toxicities through genetic or pharmacological targeting of USP22^{16,18}. However, as noted, USP22 also regulates FoxP3 transcription. In our study, we most fascinatingly observed modest decrease in only FoxP3 protein level by direct chemical perturbation is sufficient for improved disease outcomes by amelioration Treg immunosuppression in our preclinical tumor mouse models in immune functional mice. Taken together, this suggests that targeting FoxP3 directly has a viable therapeutic window. More importantly, FD03 treatment did not reduce tumor growth in mice without T cells including Treg cells, clearly indicating that FD03 achieves its tumor-suppressive activity through boosting antitumor immunity. Furthermore, following FD03 treatment, we observed amelioration of Treg immunosuppression in the tumor microenvironment through multiple key suppressive markers in Treg cells, highlighting its capacity to simultaneously disrupt several axes of Treg function through a single small molecule intervention. These results underscore the promise of chemically induced FoxP3 degradation as a novel and tractable strategy for directly targeting Treg function in cancer immunotherapy.

### Methods

Compounds. Lead compounds (FD01, FD02, FD03 (IUPAC: 1-[4-(3-cyclohexylphenyl)sulfonylpiperazin-1-yl]prop-2-en-1-one), FD04, FD05) were resynthesized by WuXi AppTec after all applications after the initial screen shown in FIG. 5b. MG132 (#1211877-36-9) and cycloheximide (CHX, #239765) were purchased from Selleckchem and Sigma-Aldrich respectively. Original compounds used for high throughput screening were a kind gift from Dr. Benjamin Cravatt.

Mice. C57BL/6J (strain no. 000664), Foxp3^{YFP-cre} (strain no. 016959), and Rag1^{tm1Mom} lymphocyte deficient mice (strain no. 002216) mice were obtained from The Jackson Laboratory. Unless otherwise noted, mice used for tumor experiments were 8-16 weeks, mice used for ex vivo experiments were aged between 8-24 weeks, and equal proportions of males and females were used. Animals received water ad libitum, were housed at a temperature range of 20° C.-23° C. under 12-h light/12-h dark cycles and received standard rodent chow. These mice were maintained and used at the Northwestern University mouse facility under pathogen-free conditions in accordance with institutional guidelines and following animal study proposals approved by the institutional animal care and use committees (IACUC).

Cell lines and primary cell culture. HEK293T cell line (ATCC #CRL-3216) and B16-F10 melanoma cell line (ATCC #CRL-6475) were purchased from American Type Culture Collection (ATCC). LentiX 293T cells (TakaraBio #632180) were purchased from TakaraBio. MC38 colon carcinoma cell line was a kind from Dr. Samuel Weinberg. MT-2 human Treg-like and MT-2 FoxP3-knockout cell lines were kind gifts from Dr. Benjamin D. Singer.

HEK293T, LentiX, and B16-F10 cells were cultured in DMEM (Cytiva #SH30243.FS) with 10% heat inactivated (HI) fetal bovine serum (FBS, R&D Systems #S11150) and 100 U/ml penicillin/streptomycin (Pen-Strep, Thermofisher Scientific #15140122) at 5% CO₂ at 37° C. MC38 cells were cultured in DMEM supplemented with 10% FBS, 100 U/ml Pen-Strep, 1× Non-essential amino acids (NEAA, Cytiva #SH30238.01), 10 mM HEPES (Corning #25-060-C1), and 1 mM sodium pyruvate (Corning #25-000-C1). MT-2 cells were cultured in RPMI (Corning #10-040-CV) with 10% FBS and 100 U/ml Pen-Strep. Primary murine and human lymphocytes were cultured in RPMI supplemented with 10% HI FBS (Cytvia #SH30071.03), 100 U/ml Pen-Strep, 5 mM HEPES, 1 mM sodium pyruvate, 2 mM L-glutamine (Cytiva #SH30034.01), 1×NEAA, and 0.05 mM b-mercaptoethanol (Sigma Aldrich #M3148-100ML) which will be referred to as T cell media (TCM).

High throughput screening by high content imaging. HEK293T cells stably expressing FoxP3-Artichoke were plated in 384-well plates and compounds were added at 5 mM concentration to each well after the cells adhered. After 16 hours of treatment, cells were stained with Hoechst Blue and imaged on the Molecular Devices ImageXpress Micro confocal high content imager. The Multi Wavelength Cell Scoring module in the MetaXpress software was used to analyze the images.

Generation and treatment of murine in vitro differentiated induced T regulatory cells. CD4+ T cells were purified using from lymphoid organs using a selection kit (Stem Cell Technologies #19852). CD4+ T cells were cultured in plates pre-coated with 3 mg/mL anti-CD3 (Thermofisher Scientific #16-0031-86) and 5 mg/mL anti-CD28 (Thermofisher Scientific #16-0281-86) in TCM supplemented with 5 ng/ml IL-2 (Thermofisher Scientific #212-12-20UG), 0.5-5 ng/mL TGFb (Thermofisher Scientific #100-21-10UG), 2 g/mL anti-IL-4 and 2 μg/mL anti-IFN-γ. After five days, cells were rested for one day, and then treated for 16-48 hours.

Generation and treatment of human in vitro differentiated T regulatory cells. Live human peripheral blood mononuclear cells were purchased from Stem Cell Technologies (Stem Cell Technologies #200-0077), and CD4+ naïve T cells were enriched by negative selection using the Easy Sep Human Naïve CD4+ T Cell Isolation Kit (Stem Cell Technologies #19555). Isolated naïve CD4+ T cells were stimulated using 2 μg/mL anti-CD3 (Biolegend #317302) and 2 μg/mL anti-CD28 (Biolegend #302902) plate-bound antibodies and differentiated into iTreg cells by addition of Human Treg Differentiation Supplement (Stem Cell Technologies #10977) and 100 IU/mL rhIL-2 (Thermofisher Scientific #200-02) to TCM for five days in TCM. The cells were then rested for one day before treatment with varying concentrations of FD03 for 16-20 hours.

In vitro T regulatory cell suppression assay. In vitro induced T regulatory cells (iTreg cells) were generated from splenic CD4+ T cells of Foxp3^{YFP-cre} mice and treated with DMSO or FD03 for 16 hours as described above. CD8+ T cells were enriched from lymphocytes isolated from spleens of C57BL/6J or Foxp3^{YFP-cre} mice using a selection kit (Stem Cell Technologies #19853). CD8+ T cells were stained with Cell Trace Violet (Thermofisher Scientific #C34557) and then plated at a density of 2.5×10⁴ cells per well using TCM in a round-bottom 96-well plate with 2.5×10⁴ CD3/CD28 activation beads (Miltenyi Biotec #130-786 095-925) and 30 IU/ml IL-2 (PeproTech #212-12). Varying ratios of Treg cells were incubated with CD8+ T cells for 72 hours then CD8+ T cell proliferation was measured by flow cytometry analysis of Cell Trace Violet dilution.

Syngeneic tumor models, treatment, and processing. Murine syngeneic cancer cell lines were passaged at least once after freeze-thaw. When cells reached 60-80% confluency, they were washed and resuspended in 100 ul of phosphate-buffered-saline (PBS, Cytiva #SH30028.3), then injected subcutaneously into the right hind flanks of mice. 2.5×10⁴ cells B16-F10 cells and 5×10⁴ cells MC38 cells were injected per mouse. Mice were monitored every 2-3 days for tumor growth. Tumor volume was measured through digital calipers along two orthogonal axes (x and y) and calculated as (xy(x+y))/4. Mice were treated by 50 mL intratumoral injection of vehicle (DMSO in PBS) or 5 mg/kg FD03 (FD03 in PBS) when tumors reached approximately 100 mm³. Mice were euthanized if tumors exceeded the 2000 mm³ tumor size limit agreed by the Institutional Review Board (IRB).

Tumors were processed into single cell suspensions as described in Chaudhuri, et al (2024) 49. After euthanasia, tumors were excised from the mice and cut finely before incubating in tumor digestion buffer containing 2 mg/ml collagenase type 4 (Worthington Biochemical #LS004189), 5 mM calcium chloride, and 1% FBS in HBSS (Thermofisher Scientific #14170120) at 37° C. for 20-30 min. Next, samples were mashed and filtered with addition of RPMI and EDTA pH 8.0 to neutralize the digestion buffer. The single cell suspension was centrifuged and red blood cells were lysed using ACK lysis buffer. Tumor infiltrating lymphocytes were then enriched from the tumor single cell suspension using the EasySep Mouse TIL (CD45) Positive Selection Kit (Stem Cell Technologies #100-0350).

Cloning and generation of FoxP3-Artichoke stable expression cell lines. FoxP3− Artichoke was constructed by inserting full length human Foxp3 complimentary DNA (cDNA) into Artichoke (Addgene plasmid #73320) at the PshAI restriction enzyme site²⁶. Lentiviruses of FoxP3-Artichoke were generated as described in Wang, et al by co-transfecting aforementioned plasmids with packaging plasmids, psPAX2 (Addgene plasmid #12260) and pMD2.G (Addgene plasmid #12259), into LentiX 293T cells (TakaraBio #632180) using jetPRIME Transfection reagent (Polyplus #117-15). Viral supernatant was harvested 72 hours after transfection⁵². HEK293T cells were transduced using the generated FoxP3-Artichoke lentivirus and 2-4 rounds of fluorescent activated cell sorting (FACS) for GFP+mCherry+ cells to generate stable expression cell lines.

Generation of CRISPR-Cas9-mediated knockout cells. MT-2 knockout cells were generated using CRISPR ribonucleoprotein (RNP) nucleofection techniques as published by Hultquist, et al (2019) 53. Cas9-sgRNA RNP complex was incubated with MT-2 cells resuspended in SE Cell Line Nucleofection Solution (Lonza Bioscience #V4XC-1012). After incubation, the mixture was transferred into the Nucleocuvette Vessel (Lonza Bioscience #V4XC-1012) and then electroporated using code CL-120 on the Lonza 4D X-Unit.

To target STUB1, the following sgRNAs (ordered from IDT) were used: STUB1 sgRNA-AA: 5′ GGCCGTGTATTACACCAACC 3′ (SEQ ID NO: 1); STUB1 sgRNA-AB: 5′ GAGTGCCAGCGAAACCACGA 3′ (SEQ ID NO: 2); STUB1 sgRNA-AF: 5′ TGGCCGTGTATTACACCAAC 3′ (SEQ ID NO: 3). Successful knockout of STUB1 was confirmed by Western blot.

Flow cytometry analysis, intracellular cytokine stimulation, and cell sorting. For flow cytometry analysis, cells were first resuspended in fluorescence-activated cell sorting (FACS) buffer consisting of PBS and 3% HI FBS (R&D Systems #S11150) and incubated with blocking CD16/32 antibody (BioLegend #101301) and Fixable Viability Dye (eBioscience #65-0863-18/#65-0866-18), then surface stained. For the detection of intracellular proteins, the FoxP3/Transcription Factor Staining Buffer Set (Thermofisher Scientific #00-5223-56) was used. For samples requiring intracellular cytokine staining, cells were first incubated for 1.5-4 hours with 25 ng/mL PMA (Sigma-Aldirch #P1585), 0.5 μM ionomycin (Sigma-Aldrich #I0634), and 10 μg/mL monensin (Thermofisher Scientific #00-4505-51).

The following antibodies coupled to different fluorochromes was used for staining at 1:50 to 1:200 ratios: CD45 (30-F11), CD4 (GK1.5), CD8 (53-6.7), CD25 (PC61), CTLA-4 (UC10-4B9), CD357/GITR (DTA-1), PD-L1 (10F.9G2), and TIGIT (GIGD7), human CD4 (OKT4), human CD3 (OKT3), human CD25 (BC96), human FoxP3 (PCH101), FoxP3 (FJK-16s), Helios (22F6), IFN-γ (XMG1.2), and GZMB (QA16A02). Antibodies were purchased from either Biolegend or Thermofisher Scientific/eBioscience. All the antibodies are specific to mouse or mouse/human.

After staining, cells were analyzed using a BD LSRFortessa Cell Analyzer (Becton Dickinson). FlowJo (Becton Dickinson) was used for analyzing flow cytometry data. For cell sorting, cells were stained as described above then resuspended in FACS buffer. Cell sorting was performed using either BD FACSAria SORP system (Becton Dickinson) or MACSQuant Tyto Cell Sorter (Miltenyi).

Cell lysis, Immunoprecipitation, and Western blot. Coimmunoprecipitation (co-IP) and Western blots were performed as previously described⁴⁶. Cells were lysed in RIPA buffer (Millipore #20-188) with protease inhibitor (Roche #61431800) and incubated at 4° C. for 30 minutes. Cells were centrifuged at a high speed to pellet cell debris. The resulting supernatant was extracted for immunoprecipitation and/or Western blot.

For co-IP, the supernatant was then pre-cleaned with Protein G Sepharose (GE Healthcare #17-0618-02) three times then incubated with FoxP3 (PCH101, Invitrogen #14-4776-37) or isotype control (Santa Cruz Biotechnology #sc-2026) overnight at 4° C. overnight. Protein G Sepharose beads were added and incubated with the supernatants for 2 hours at 4° C. on the following day. The beads were then washed five times with RIPA buffer then incubated with 4× NuPAGE sample buffer (Thermofisher Scientific #NP0008) for 15-20 minutes at room temperature. The beads were removed by centrifugation in Pierce Spin Cups (Thermofisher Scientific #69702), and the supernatant is then prepared for Western blot.

To prepare the samples for PAGE gel, if the supernatant consisted of RIPA buffer only, 4× NuPAGE sample buffer was added. The lysate mixtures were then boiled at 95° C. for 10-15 minutes and loaded onto PAGE gels. Gels were transferred to nitrocellulose membranes and blocked in 5% non-fat milk for 1 hour or more. Membranes were then incubated with primary antibodies including: mouse FoxP3 (Cell Signaling Technology #12653), human FoxP3 (Cell Signaling Technology #5298), Vinculin (Cell Signaling Technology #13901), STUB1/CHIP (Cell Signaling Technology #2080), mCherry (Proteintech #26765-1-AP), GFP (Cell Signaling Technology #2956), Actin (Proteintech #66009-1-Ig), USP22 (Invitrogen #PA5-12027), USP21 (Invitrogen #PA570017), USP7/HAUSP (Abcam #ab239936), and poly-ubiquitin (Cell Signaling Technology #3936). Next, membranes were incubated with corresponding secondary antibodies, Goat HRP-linked anti-Rabbit IgG (Millipore Sigma #12-348) or Goat HRP-linked anti-Mouse IgG (Millipore Sigma #12-349), for 1 hour at room temperature. Membranes were then imaged using enhanced chemiluminescence substrate (Thermofisher Scientific #34577) to visualize signals on the BioRad ChemiDoc XRS imaging system. If necessary, membranes were stripped with Restore Western Blot stripping buffer (Thermofisher Scientific #21059), to repeat probing for another protein of interest. Image analysis and quantification was performed using FIJI/ImageJ.

### Example 2—Association of High FoxP3+ Immune Suppressive Treg Infiltration with Poor Clinical Outcomes in Multiple Types of Human Cancers

It has been well established that high FoxP3+ regulatory T cells (Tregs) are associated with poor clinical outcomes in cancer, as they contribute to an immunosuppressive tumor microenvironment, hindering anti-tumor immune responses and promoting disease progression. Since FoxP3 is exclusively expressed in regulatory T cells (Tregs) and its expression level is closely linked to Treg suppressive functions, FoxP3 gene expression often serves as a precise indicator of tumoral Treg infiltration. We analyzed the potential of FoxP3 as a biomarker for predicting clinical outcomes across all cancer types listed in the TCGA database. Our findings reveal that high FoxP3 expression is associated with poor clinical outcomes in 13 out of 33 cancer types (Table 1), suggesting that targeting FoxP3 holds significant potential for enhancing antitumor immunity in the treatment of these cancers. Additionally, while Treg infiltration is high in many human cancers, a simple median-based high vs. low stratification did not yield a significant association with clinical outcomes. Nevertheless, FoxP3-targeted strategies could presumably enhance antitumor immunity in these tumors as well.

TABLE 1
Association of FoxP3 expression with clinical outcomes.
Statistically significant results are bolded.

| Tumor | p value | Full name |
|---|---|---|
| ACC | 0.00046 | Adrenocortical carcinoma |
| BLCA | 0.86 | Bladder Urothelial Carcinoma |
| BRCA | 0.6 | Breast invasive carcinoma |
| CESC | 0.0018 | Cervical squamous cell |
| | | carcinoma and endocervical |
| | | adenocarcinoma |
| CHOL | 0.18 | Cholangiocarcinoma |
| COAD | 0.17 | Chronic Myelogenous Leukemia |
| DLBC | 0.62 | Colon adenocarcinoma |
| ESCA | 0.26 | Esophageal carcinoma |
| GBM | 0.0002 | Glioblastoma multiforme |
| HNSC | 0.015 | Head and Neck squamous cell |
| | | carcinoma |
| KICH | 0.65 | Kidney Chromophobe |
| KIRC | 0.00014 | Kidney renal clear cell carcinoma |
| KIRP | 0.021 | Kidney renal papillary cell |
| | | carcinoma |
| LAML | 0.0041 | Acute Myeloid Leukemia |
| LGG | 0.54 | Brain Lower Grade Glioma |
| LIHC | 0.6 | Liver hepatocellular carcinoma |
| LUAD | 0.002 | Lung adenocarcinoma |
| LUSC | 0.13 | Lung squamous cell carcinoma |
| MESO | 0.8 | Mesothelioma |
| OV | 0.003 | Ovarian serous |
| | | cystadenocarcinoma |
| PAAD | 0.49 | Pancreatic adenocarcinoma |
| PCPG | 0.8 | Pheochromocytoma and |
| | | Paraganglioma |
| PRAD | 0.7 | Prostate adenocarcinoma |
| READ | 0.03 | Rectum adenocarcinoma |
| SARC | 0.15 | Sarcoma |
| SKCM | 0.0012 | Skin Cutaneous Melanoma |
| STAD | 0.37 | Stomach adenocarcinoma |
| PGCT | 0.9 | Testicular Germ Cell Tumors |
| THCA | 0.8 | Thyroid carcinoma |
| THYM | 0.011 | Thymoma |
| UCEC | 0.32 | Uterine Corpus Endometrial |
| | | Carcinoma |
| UCS | 0.77 | Uterine Carcinosarcoma |
| UVM | 0.004 | Uveal Melanoma |

### Example 3—CRISPR Screening Identifies Possible Interactions Between E3 Ligases and FoxP3 Mediated by FD03

To identify how which E3 ligases may be mediating degradation induced by FD03, we performed a targeted CRISPR screen in FoxP3-Artichoke expressing HEK293T cells. FoxP3-Artichoke expressing HEK293T cells were transduced with sgRNA guide library targeting 600 E3 ligases alongside guides targeting GFP and non-targeting guides. After transduction and selection, we treated cells for 16 hours using FD03 then sorted the cells with high GFP expression for sequencing (FIG. 8A). This screen allows us to identify E3 ligases which either 1. Have increased degradation of FoxP3 induced by FD03 and are endogenous E3 ligases; 2. degrade FoxP3 dependent on FD03 and are not endogenous E3 ligases of FoxP3; or 3. degrade FoxP3 as endogenous E3 ligases independent from FD03.

From this screen, we identified guides targeting several E3 ligases which were enriched in the sorted population compared to the unsorted or total population of cells post FDO3-treatment. These E3 ligases include UBE3C, SIAH1, RNF113A, FBWX7, and HUWE1. We are currently following up possible hits on UBE3C, RNF113A, and FBXW7 by generation of knockout MT-2 cells and assessing FD03 degradation ability of FoxP3 in the knockout cell lines. We will also follow up with these E3 ligases by assessing whether they are endogenous E3 ligases of FoxP3 and whether FD03 treatment changes their interactions with FoxP3.

### REFERENCES

1 Rudensky, A. Y. Regulatory T cells and Foxp3. Immunol Rev 241, 260-268 (2011). doi.org: 10.1111/j.1600-065X.2011.01018.x
2 Beyer, M. & Schultze, J. L. Regulatory T cells in cancer. Blood 108, 804-811 (2006). doi.org: 10.1182/blood-2006-02-002774
3 Dejaco, C., Duftner, C., Grubeck-Loebenstein, B. & Schirmer, M. Imbalance of regulatory T cells in human autoimmune diseases. Immunology 117, 289-300 (2006). doi.org: 10.1111/j.1365-2567.2005.02317.x
4 Chen, B. J., Zhao, J. W., Zhang, D. H., Zheng, A. H. & Wu, G. Q. Immunotherapy of Cancer by Targeting Regulatory T cells. Int Immunopharmacol 104, 108469 (2022). doi.org: 10.1016/j.intimp.2021.108469
5. Dees, S., Ganesan, R., Singh, S. & Grewal, I. S. Regulatory T cell targeting in cancer: Emerging strategies in immunotherapy. Eur J Immunol 51, 280-291 (2021). doi.org: 10.1002/eji.202048992
6 Chaudhry, A. et al. Interleukin-10 signaling in regulatory T cells is required for suppression of Th17 cell-mediated inflammation. Immunity 34, 566-578 (2011). doi.org: 10.1016/j.immuni.2011.03.018
7 Curiel, T. J. Tregs and rethinking cancer immunotherapy. J Clin Invest 117, 1167-1174 (2007). doi.org: 10.1172/JCI31202
8 Kamada, T. et al. PD-1 (+) regulatory T cells amplified by PD-1 blockade promote hyperprogression of cancer. Proc Natl Acad Sci USA 116, 9999-10008 (2019). doi.org: 10.1073/pnas.1822001116
9 Qin, F. X. Dynamic behavior and function of Foxp3+ regulatory T cells in tumor bearing host. Cell Mol Immunol 6, 3-13 (2009). doi.org: 10.1038/cmi.2009.2
10 Dougan, M. & Dranoff, G. The immune response to tumors. Curr Protoc Immunol Chapter 20, Unit 20 11 (2009). doi.org: 10.1002/0471142735.im2011s85
11 Dougan, M. & Dranoff, G. Immune therapy for cancer. Annual review of immunology 27, 83-117 (2009). doi.org: 10.1146/annurev.immunol.021908.132544
12 Zitvogel, L., Tesniere, A. & Kroemer, G. Cancer despite immunosurveillance: immunoselection and immunosubversion. Nature reviews. Immunology 6, 715-727 (2006). doi.org: 10.1038/nri1936
13 Ke, X. et al. Roles of CD4+CD25 (high) FOXP3+ Tregs in lymphomas and tumors are complex. Front Biosci 13, 3986-4001 (2008).
14 Ramsdell, F. & Rudensky, A. Y. Foxp3: a genetic foundation for regulatory T cell differentiation and function. Nat Immunol 21, 708-709 (2020). doi.org: 10.1038/s41590-020-0694-5
15 Zheng, Y. & Rudensky, A. Y. Foxp3 in control of the regulatory T cell lineage. Nat Immunol 8, 457-462 (2007). doi.org: 10.1038/ni1455
16 Cortez, J. T. et al. CRISPR screen in regulatory T cells reveals modulators of Foxp3. Nature 582, 416-420 (2020). doi.org: 10.1038/s41586-020-2246-4
17 Williams, L. M. & Rudensky, A. Y. Maintenance of the Foxp3-dependent developmental program in mature regulatory T cells requires continued expression of Foxp3. Nat Immunol 8, 277-284 (2007). doi.org: 10.1038/ni1437
18 Montauti, E. et al. A deubiquitination module essential for T (reg) fitness in the tumor microenvironment. Sci Adv 8, eabo4116 (2022). doi.org: 10.1126/sciadv.abo4116
19 Hanzl, A. & Winter, G. E. Targeted protein degradation: current and future challenges. Curr Opin Chem Biol 56, 35-41 (2020). doi.org: 10.1016/j.cbpa.2019.11.012
20 Cowan, A. D. & Ciulli, A. Driving E3 Ligase Substrate Specificity for Targeted Protein Degradation: Lessons from Nature and the Laboratory. Annu Rev Biochem 91, 295-319 (2022). doi.org: 10.1146/annurev-biochem-032620-104421
21 Tsai, J. M., Nowak, R. P., Ebert, B. L. & Fischer, E. S. Targeted protein degradation: from mechanisms to clinic. Nat Rev Mol Cell Biol 25, 740-757 (2024). doi.org: 10.1038/s41580-024-00729-9
22 Teng, M. & Gray, N. S. The rise of degrader drugs. Cell Chem Biol 30, 864-878 (2023). doi.org: 10.1016/j.chembiol.2023.06.020
23 Bekes, M., Langley, D. R. & Crews, C. M. PROTAC targeted protein degraders: the past is prologue. Nat Rev Drug Discov 21, 181-200 (2022). doi.org: 10.1038/s41573-021-00371-6
24 Slabicki, M. et al. The CDK inhibitor CR8 acts as a molecular glue degrader that depletes cyclin K. Nature 585, 293-297 (2020). doi.org: 10.1038/s41586-020-2374-x
25 Chen, Z. et al. The ubiquitin ligase Stub1 negatively modulates regulatory T cell suppressive activity by promoting degradation of the transcription factor Foxp3. Immunity 39, 272-285 (2013). doi.org: 10.1016/j.immuni.2013.08.006
26 Hamano, R., Wu, X., Wang, Y., Oppenheim, J. J. & Chen, X. Characterization of MT-2 cells as a human regulatory T cell-like cell line. Cell Mol Immunol 12, 780-782 (2015). doi.org: 10.1038/cmi.2014.123
27 Mccullough, M. J. et al. Characterization of the MT-2 Treg-like cell line in the presence and absence of forkhead box P3 (FOXP3). Immunol Cell Biol 102, 211-224 (2024). doi.org: 10.1111/imcb.12725
28 van Loosdregt, J. et al. Stabilization of the transcription factor Foxp3 by the deubiquitinase USP7 increases Treg-cell-suppressive capacity. Immunity 39, 259-271 (2013). doi.org: 10.1016/j.immuni.2013.05.018
29 Yang, J. et al. The deubiquitinase USP44 promotes Treg function during inflammation by preventing FOXP3 degradation. EMBO Rep 21, e50308 (2020). doi.org: 10.15252/embr.202050308
30 Amoozgar, Z. et al. Targeting Treg cells with GITR activation alleviates resistance to immunotherapy in murine glioblastomas. Nat Commun 12, 2582 (2021). doi.org: 10.1038/s41467-021-22885-8
31 Ke, S. et al. High-level of intratumoral GITR+CD4 T cells associate with poor prognosis in gastric cancer. iScience 25, 105529 (2022). doi.org: 10.1016/j.isci.2022.105529
32 Iglesias-Escudero, M., Arias-Gonzalez, N. & Martinez-Caceres, E. Regulatory cells and the effect of cancer immunotherapy. Mol Cancer 22, 26 (2023). doi.org: 10.1186/s12943-023-01714-0
33 Togashi, Y., shitara, K. & Nishikawa, H. Regulatory T cells in cancer immunosuppression-implications for anticancer therapy. Nat Rev Clin Oncol 16, 356-371 (2019). doi.org: 10.1038/s41571-019-0175-7
34 Cai, L., Li, Y., Tan, J., Xu, L. & Li, Y. Targeting LAG-3, TIM-3, and TIGIT for cancer immunotherapy. J Hematol Oncol 16, 101 (2023). doi.org: 10.1186/s13045-023-01499-1
35 Kurtulus, S. et al. TIGIT predominantly regulates the immune response via regulatory T cells. J Clin Invest 125, 4053-4062 (2015). doi.org: 10.1172/JCI81187
36 Lang, Y. et al. TIGIT Blockade Reshapes the Tumor Microenvironment Based on the Single-cell RNA-Sequencing Analysis. J Immunother 47, 172-181 (2024). doi.org: 10.1097/CJI.0000000000000511
37 Yang, Z. Z. et al. TIGIT Expression Is Associated with T-cell Suppression and Exhaustion and Predicts Clinical Outcome and Anti-PD-1 Response in Follicular Lymphoma. Clin Cancer Res 26, 5217-5231 (2020). doi.org: 10.1158/1078-0432.CCR-20-0558
38 Zhang, P. et al. Targeting TIGIT for cancer immunotherapy: recent advances and future directions. Biomark Res 12, 7 (2024). doi.org: 10.1186/s40364-023-00543-z
39 Zhulai, G. & Oleinik, E. Targeting regulatory T cells in anti-PD-1/PD-L1 cancer immunotherapy. Scand J Immunol 95, e13129 (2022). doi.org: 10.1111/sji.13129
40 O'Callaghan, D. S. et al. Tumour islet Foxp3+ T-cell infiltration predicts poor outcome in nonsmall cell lung cancer. Eur Respir J 46, 1762-1772 (2015). doi.org: 10.1183/13993003.00176-2014
41 Revenko, A. et al. Direct targeting of FOXP3 in Tregs with AZD8701, a novel antisense oligonucleotide to relieve immunosuppression in cancer. J Immunother Cancer 10 (2022). doi.org: 10.1136/jitc-2021-003892
42 Casares, N. et al. A peptide inhibitor of FOXP3 impairs regulatory T cell activity and improves vaccine efficacy in mice. J Immunol 185, 5150-5159 (2010). doi.org: 10.4049/jimmunol. 1001114
43 Wang, Y. et al. Stimulation of antitumor immunity by FoxP3-targeting PROTAC. Biomed Pharmacother 163, 114871 (2023). doi.org: 10.1016/j.biopha.2023.114871
44 Gregory, S. et al. The ubiquitin-specific peptidase 22 is a deubiquitinase of CD73 in breast cancer cells. Am J Cancer Res 12, 5564-5575 (2022).
45 Lin, Z. et al. USP22 antagonizes p53 transcriptional activation by deubiquitinating Sirt1 to suppress cell apoptosis and is required for mouse embryonic development. Mol Cell 46, 484-494 (2012). doi.org: 10.1016/j.molcel.2012.03.024
46 Melo-Cardenas, J. et al. USP22 deficiency leads to myeloid leukemia upon oncogenic Kras activation through a PU.1-dependent mechanism. Blood 132, 423-434 (2018). doi.org: 10.1182/blood-2017-10-811760
47 Chaudhuri, S. M. et al. Mediator complex subunit 1 architects a tumorigenic Treg cell program independent of inflammation. Cell Rep Med 5, 101441 (2024). doi.org: 10.1016/j.xcrm.2024.101441
48 Tay, C., Tanaka, A. & Sakaguchi, S. Tumor-infiltrating regulatory T cells as targets of cancer immunotherapy. Cancer Cell 41, 450-465 (2023). doi.org: 10.1016/j.ccell.2023.02.014
49 Bonazzi, S. et al. Discovery and characterization of a selective IKZF2 glue degrader for cancer immunotherapy. Cell Chem Biol (2023). doi.org: 10.1016/j.chembiol.2023.02.005
50 Wang, S. et al. CRISPR screening identifies the deubiquitylase ATXN3 as a PD-L1-positive regulator for tumor immune evasion. J Clin Invest 133 (2023). doi.org: 10.1172/JCI167728
51 Hultquist, J. F. et al. CRISPR-Cas9 genome engineering of primary CD4 (+) T cells for the interrogation of HIV-host factor interactions. Nat Protoc 14, 1-27 (2019). doi.org: 10.1038/s41596-018-0069-7
Schoenhals, J. E. et al. Anti-glucocorticoid-induced Tumor Necrosis Factor-Related Protein (GITR) Therapy Overcomes Radiation-Induced Treg Immunosuppression and Drives Abscopal Effects. Front Immunol 9, 2170 (2018). doi.org: 10.3389/fimmu.2018.02170
Li, T. et al. TGFBR2 mutation predicts resistance to immune checkpoint inhibitors in patients with non-small cell lung cancer. Ther Adv Med Oncol 13, 17588359211038477 (2021). doi.org: 10.1177/17588359211038477
Li, Y. et al. Dual targeting of TIGIT and PD-1 with a novel small molecule for cancer immunotherapy. Biochem Pharmacol 223, 116162 (2024). doi.org: 10.1016/j.bcp.2024.116162
Nalawansha, D. A. & Crews, C. M. PROTACs: An Emerging Therapeutic Modality in Precision Medicine. Cell Chem Biol 27, 998-1014 (2020). doi.org: 10.1016/j.chembiol.2020.07.020
Rana, S., Mallareddy, J. R., Singh, S., Boghean, L. & Natarajan, A. Inhibitors, PROTACs and Molecular Glues as Diverse Therapeutic Modalities to Target Cyclin-Dependent Kinase. Cancers (Basel) 13 (2021). doi.org: 10.3390/cancers13215506

### SEQUENCES

| SEQ | | |
|---|---|---|
| ID | | |
| NO | Description | Sequence |
| 1 | STUB1 | GGCCGTGTATTACACCAACC |
| | sgRNA-AA: | |
| 2 | STUB1 | GAGTGCCAGCGAAACCACGA |
| | sgRNA-AB | |
| 3 | STUB1 | TGGCCGTGTATTACACCAAC |
| | sgRNA-AF | |
| 4 | Human | MPNPRPGKPSAPSLALGPSPGASPSWRAAPKASDLLGARGPGGT |
| | FoxP3 | FQGRDLRGGAHASSSSLNPMPPSQLQLPTLPLVMVAPSGARLGP |
| | | LPHLQALLQDRPHFMHQLSTVDAHARTPVLQVHPLESPAMISLT |
| | | PPTTATGVFSLKARPGLPPGINVASLEWVSREPALLCTFPNPSAP |
| | | RKDSTLSAVPQSSYPLLANGVCKWPGCEKVFEEPEDFLKHCQA |
| | | DHLLDEKGRAQCLLQREMVQSLEQQLVLEKEKLSAMQAHLAG |
| | | KMALTKASSVASSDKGSCCIVAAGSQGPVVPAWSGPREAPDSLF |
| | | AVRRHLWGSHGNSTFPEFLHNMDYFKFHNMRPPFTYATLIRWA |
| | | ILEAPEKQRTLNEIYHWFTRMFAFFRNHPATWKNAIRHNLSLHK |
| | | CFVRVESEKGAVWTVDELEFRKKRSQRPSRCSNPTPGP |

1. A method of treating a disease or disorder associated with forkhead box protein P3 (FoxP3) expression in a subject in need thereof, the method comprising administering a small molecule FoxP3 degrader to the subject, wherein the small molecule FoxP3 degrader has a formula of

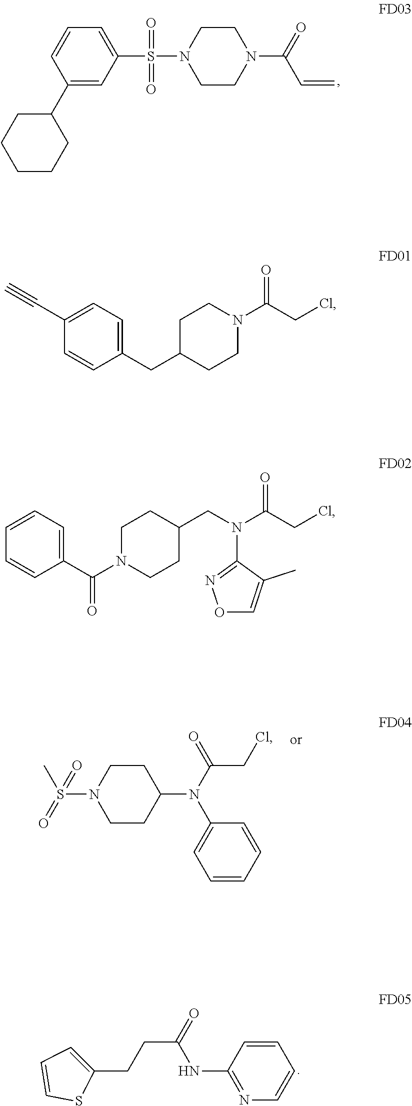

2. The method of claim 1, wherein the small molecule FoxP3 degrader has a formula of

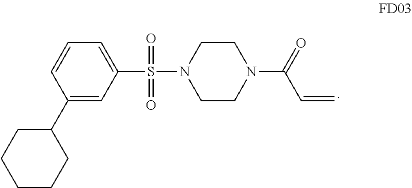 [SMILES: C=CC(=O)N1CCN(S(=O)(=O)c2cccc(C3CCCCC3)c2)CC1]

3. The method of claim 1, wherein the disease or disorder associated with FoxP3 expression comprises a cell proliferative disease or disorder or a chronic viral infection.

4. The method of claim 3, wherein the cell proliferative disease or disorder comprises cancer.

5. The method of claim 4, wherein the cancer comprises cutaneous melanoma, uveal melanoma, rectal adenocarcinoma, adrenocortical carcinoma, cervical squamous cell carcinoma, endocervical adenocarcinoma, glioblastoma multiforme, head and neck squamous cell carcinoma, renal clear cell carcinoma, renal papillary cell carcinoma, acute myeloid leukemia, lymphoma, lung adenocarcinoma, ovarian serous cystadenocarcinoma, or thymoma.

6. The method of claim 5, wherein the cancer is melanoma, lung carcinoma, lymphoma, or colorectal cancer.

7. The method of claim 6, wherein the cancer is cutaneous melanoma.

8. The method of claim 5, wherein administering comprises intravenous, intraperitoneal, peritumoral, or intratumoral administration.

9. The method of claim 3, wherein the chronic viral infection comprises human immunodeficiency virus (HIV) infection, hepatitis B virus (HBV) infection, hepatitis C virus (HCV) infection, or human papillomavirus (HPV) infection.

10. The method of claim 1, wherein the method further comprises administering an immunotherapy to the subject.

11. The method of claim 10, wherein the immunotherapy comprises an immune checkpoint inhibitor (ICI).

12. The method of claim 1, wherein the method decreases T regulatory cell (Treg) FoxP3 protein or decreases glucocorticoid-induced TNFR-related protein (GITR) expression.

13. The method of claim 12, wherein the expression of FoxP3 or GITR is decreased in T regulatory (Treg) cells.

14. The method of claim 1, wherein the method increases frequency of interferon gamma positive (IFN-γ⁺) CD4 T cells in the subject.

15. A method of inducing degradation of forkhead box protein P3 (FoxP3) in a cell, the method comprising contacting the cell with an effective amount of a small molecule FoxP3 degrader, wherein the small molecule FoxP3 degrader has a formula of

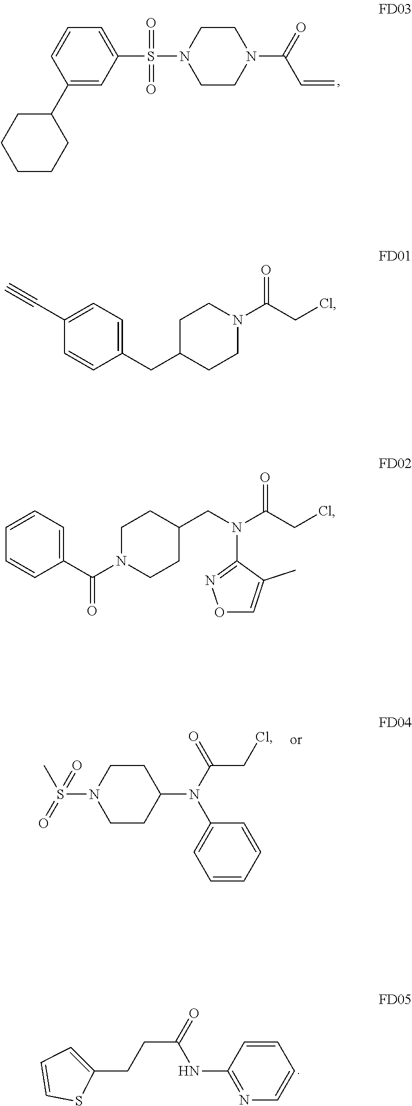

16. The method of claim 15, wherein the small molecule FoxP3 degrader has a formula of

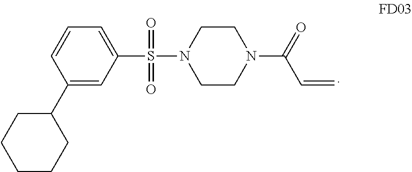 [SMILES: C=CC(=O)N1CCN(S(=O)(=O)c2cccc(C3CCCCC3)c2)CC1]

17. The method of claim 15, wherein the cell is a T regulatory cell (Treg).

18. The method of claim 17, wherein the Treg is contacted with the small molecule FoxP3 degrader in the presence of interleukin 1β (IL-1β), lipopolysaccharide (LPS), or IFN-γ.

19. The method of claim 17, wherein the Treg is a CD4 T cell.

20. The method of claim 15, wherein the method decreases glucocorticoid-induced TNFR-related protein (GITR) expression in the cell.


SEQUENCE LISTING (4 sequences)

SEQ ID NO: 1 - DNA, 20 bp, synthetic construct
  source 1..20: mol_type = other DNA
ggccgtgtattacaccaacc

SEQ ID NO: 2 - DNA, 20 bp, synthetic construct
  source 1..20: mol_type = other DNA
gagtgccagcgaaaccacga

SEQ ID NO: 3 - DNA, 20 bp, synthetic construct
  source 1..20: mol_type = other DNA
tggccgtgtattacaccaac

SEQ ID NO: 4 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP